(19) United States
(12) Patent Application Publication
(10) Pub. No.: US 2002/0177138 A1
(43) Pub. Date: Nov. 28, 2002

(54) Methods for the indentification of textual and physical structured query fragments for the analysis of textual and biopolymer information

(75) Inventor: Robert J. Boissy, Port Coquitlam (CA)

Correspondence Address: FITCH EVEN TABIN AND FLANNERY, 120 SOUTH LA SALLE STREET, SUITE 1600, CHICAGO, IL, 60603-3406, US

(73) Assignee: THE UNITED STATES OF AMERICA , represented by the Secretary, Department of Health and Human Services

(21) Appl. No.: 09/991,013

(22) Filed: Nov. 14, 2001

Related U.S. Application Data
(60) Provisional application No. 60/248,541, filed on Nov. 15, 2000.

(51) Int. Cl. C12Q 1/68; G06F 19/00; G01N 33/48; G01N 33/50

(52) U.S. Cl. 435/6; 702/20

(57) ABSTRACT

We disclose a combinatorial, hierarchical process that uses “process-patterns” in one preferred embodiment to identify, classify, and compare substrings within strings; and in another preferred embodiment to identify, classify, compare, generate, and separate fragments derived from one or more physical samples of polynucleotides. These substrings (and their physical polynucleotide counterparts) are called “partition” fragments, and the process-pattern-defined derivatives that some, but not all, “partition” fragments may yield are called “structured query fragments” (SQFs). A process-pattern is both: (i) an ordered set of short “target” (one from each major search class) sites that must be present (and whose higher-ranked members of the same major search class must not have any sites) within the relevant search area of a partition fragment, and (ii) a step-wise delimitation process (where each step has a defined polarity and occurs after a target is found) that restricts the region of a partition fragment where the next class-specific, pre-emptive target-search takes place. In one preferred embodiment, the computer software disclosed herein locates the process-patterns and SQFs of interest within the partition fragments in the string(s) under study (e.g., a set of polynucleotide sequence data), stores the results, and provides for access to this data by database query and analysis tools. These computational analyses are emulated by another preferred embodiment using physical samples of polynucleotides and the laboratory methods disclosed herein. In the latter, sequence-specific, double-stranded cleavage effectors utilize as substrates and generate as products progressively expanding sets of asymmetrically end-immobilized DNA, a process that ultimately yields extremely large numbers of individually distinguishable SQFs (called “ranged” SQFs) with lengths between 100-700 nucleotides. In almost all cases, the known process-pattern and observed length of an experimentally obtained ranged SQF provide sufficient information for the computer software disclosed herein to map the ranged SQF automatically to its partition fragment (and location) within a set of polynucleotide sequence data that characterizes the physical sample(s) of polynucleotides under study.

### GOVERNMENT SUPPORT

Work for the following disclosed invention was supported by various grants to Dr. Douglas A. Bell from the Division of Intramural Research of the National Institute of Environmental Health Sciences, a branch of the National Institutes of Health of the Department of Health and Human Services. The Government may have certain rights in the invention. Mention herein of commercial products, processes, or services is for documentation purposes only, and should not be construed as implying that the United States Government endorses or recommends any of the said commercial products, processes, or services. Any trademarks or registered trademarks mentioned is herein are the property of their respective owners.

The attached 652 page computer program code printout is part of the specification and hereby incorporated by reference into the specification.

### FIELD OF THE INVENTION

The invention relates to computational and laboratory methods and databases for analyzing textual and biological sequence information. More specifically, the present invention provides methods for characterizing text strings, including text strings representing biopolymer information, and methods for characterizing a physical sample or samples of the biopolymers that the text strings represent.

### BACKGROUND

The essential information in a biopolymer such as DNA, RNA, or protein can be represented by a “primary sequence” (where the adjective “primary” is often omitted) that is simply a string of characters with a defined polarity. Each character in such a string must be a member of a small set of characters (where each character in the set represents one of the structural and information-bearing monomer units that may be found in the biopolymer molecule), and the polarity of such a string reflects the chemical nature of the bond formed between each successive monomer.

There is a massive and rapidly growing amount of data in biopolymer sequence databases. Computational analysis of biopolymer sequence data enables the identification of substrings therein that may represent regions with functional, conformational, or regulatory significance (e.g., open-reading frames, palindromes, or promoters in DNA). Computational analysis of biopolymer sequence homology is especially important, because sequence homologues often contain or encode similar functional properties. Recent reviews of computational approaches for analyzing biopolymer sequence data include: (i) Baldi, P., et al.; Bioinformatics: The Machine Learning Approach; MIT Press: Cambridge Mass., USA, 1998; (ii) Biological Sequence Analysis: Probabilistic Models of Proteins and Nucleic Acids; Durbin, R., Ed.; Cambridge University Press: Cambridge, UK, 1999; (iii) Gusfield, D.; Algorithms on Strings, Trees, and Sequences: Computer Science and Computational Biology; Cambridge University Press: Cambridge, UK, 1997; and (iv) Salzberg, S. L., et al., Eds.; Computational Methods in Molecular Biology; New Comprehensive Biochemistry, Vol. 32; Elsevier: Amsterdam, 1999.

Although sequence homologues often contain or encode similar functional properties, there are cases where biopolymers whose sequences contain or encode similar functional properties have divergent sequences. The identification, classification, comparison, and establishment of phylogenetic relationships among such sequences are challenging computational problems. The pertinent computational prior art for the analysis of biopolymer sequence data cannot always answer these and other important questions (e.g., the identification of gene regulatory regions). Therefore, there remains a need for a flexible computational method that efficiently compares sequence information to identify related sequences. Additionally, there is a need for a computational method for comparing sequences and identifying sequence patterns, wherein the method can be emulated in the laboratory.

It is a well-recognized problem that there are errors in the polynucleotide sequence data submitted to sequence databases (Pennisi, E.; Science 1999, 286, 447-450). These errors may in some cases simply reflect mistakes in the acquisition, assembly, and reporting of data by submitters. In other cases, however, sequence data that had been accurately obtained, assembled and reported may contain errors because a cloned DNA insert had undergone a mutational event (e.g., insertion, deletion or rearrangement) during recombinant DNA cloning. Therefore, there remains a need for effective methods of identifying polynucleotide sequence database errors.

Knowledge of the sequence of a biopolymer (or a part thereof) enables a variety of useful preparative and analytical laboratory procedures. Those developed for studies of polynucleotides often include as an important step the synthesis of locus-specific oligonucleotides for use as either: (i) primers for the enzymatic amplification of a specific fragment by such methods as the polymerase chain reaction (PCR); or (ii) hybridization probes for the identification or analysis of a specific fragment. The PCR is described in U.S. Pat. Nos. 4,683,195; 4,683,202; and 4,800,159; and a review of current applications using this technique is found in PCR Applications: Protocols For Functional Genomics; Innis, M. A., Ed.; Academic Press: San Diego, 1999. In general, in either an analytical or a preparative mode, the very specificity of the PCR and related amplification techniques presents a significant scalability barrier (i.e., a barrier to using the procedure for the analysis or preparative isolation of large numbers of specific fragments of interest). This is because a set of two locus-specific primers is required for the amplification of each fragment, and the PCR amplification conditions required for the function of different sets of PCR primers may be incompatible, imposing limits on the number of specific fragments that can be produced by multiplex PCR reactions. This scalability problem affects not only the PCR and related amplification techniques, but also a variety of other important procedures that rely on them. Examples of the latter include the procedures disclosed in U.S. Pat. Nos. 5,837,832; 5,858,659; and 5,925,525 for the analysis of DNA sequence variation (e.g., in human genomic DNA) using high-density, micro-fabricated arrays of specific oligonucleotides.

U.S. Pat. No. 5,599,696 describes “ligation-mediated, single-sided PCR”, a variation of the PCR technique that essentially requires only one locus-specific primer for amplification of a specific polynucleotide fragment of undefined nucleotide sequence. A synthetic annealing site required for a “generic” second amplification primer is introduced artificially by the ligation of double-stranded oligonucleotide adapters (see U.S. Pat. No. 4,321,365) of known sequence on one end of the fragment(s) to be amplified. Although very useful in some applications, the use of only one locus-specific primer and a second “generic” primer in ligation-mediated, single-sided PCR cannot fully address the scalability problem associated with conventional implementations of PCR and related amplification techniques. Therefore, there remains a need for polynucleotide amplification methods that allow the analysis or preparative isolation of large numbers of specific fragments of interest without using any locus-specific primers.

Complementary polynucleotide strands are capable of stable, precise, sequence-specific hybridization under appropriate conditions. Polynucleotide hybridization reactions are reviewed in Cantor, C. R.; Smith, C. L. Genomics: The Science and Technology Behind the Human Genome Project; Wiley: New York, 1999. Polynucleotide hybridization probes are typically either synthetic oligonucleotides, or a molecular clone or a fragment derived thereof, and may be used in solution or immobilized on a solid support. In general, polynucleotide hybridization probes have a known sequence or sequence repertoire (e.g., degenerate probes) that is ultimately derived from either: (i) a reverse translation of a protein sequence; or (ii) known polynucleotide sequence data determined from a recombinant DNA clone. However, it is difficult if not impossible to determine what percentage of a complex genome (e.g., the human genome) may either not be clonable, or if clonable experience a mutational event (e.g., insertion, deletion or rearrangement) during recombinant DNA cloning. Therefore, there remains a need to identify methods for polynucleotide sequence analysis that do not require cloning of a polynucleotide.

Obtaining a comprehensive set of polynucleotide hybridization probes for transcript mapping and quantitative analyses of gene expression is further complicated by the difficulty of obtaining a correspondingly comprehensive set (library) of clonable complementary DNA (cDNA) inserts derived from messenger RNA. The latter task is difficult because various parameters (e.g., cell type, stage of development, disease state, or environmental exposure) can influence gene expression. Thus, gene-expression array systems such as described in U.S. Pat. No. 5,807,522 are limited by the availability of cDNAs, and as a result may have difficulty obtaining a complete survey of changes in gene expression. As an example, it would be difficult for such a cDNA-based expression array system to survey completely the set of otherwise silent genes whose expression is induced in response to a disease state or environmental exposure, because some of these genes may not be represented in any cDNA library.

Knowledge of the sequence of a biopolymer (or a part thereof) also enables the identification of similarities and differences in the sequence of a specific region of a biopolymer using physical samples (e.g., DNA) obtained from different individuals. Many genetic studies use DNA sequence information from related individuals (in linkage studies) or otherwise comparable groups of unrelated individuals (in association studies) to identify regions of sequence identity (lack of variation) in individuals concordant for a trait, or regions of sequence variation in individuals discordant for a trait. These important studies are reviewed in: Ott, J. Analysis of Human Genetic Linkage, 3rd ed.; Johns Hopkins University Press: Baltimore, 1999; Approaches to Gene Mapping in Complex Human Diseases; Haines, J. L., Pericak-Vance, M. A., Eds.; Wiley-Liss: New York, 1998; Donnis-Keller, H. Human Gene Mapping Techniques; Stockton Press: London, 1999. Some important issues that these studies face are: (i) dissecting complex traits (mapping multiple genetic loci that may contribute to the same phenotype); (ii) the identification of large numbers of polymorphic sites throughout the human genome; and (iii) the determination (“scoring”) of the genotype at each of a large number of polymorphic sites throughout the human genome in physical samples of polynucleotides obtained from many subjects. Unfortunately, the laboratory methods currently in use to address these important issues generally suffer from limited scalability. Therefore, there remains a need for scalable laboratory methods for identifying DNA sequence variants in individuals discordant for a trait, and regions of DNA sequence identity in individuals concordant for a trait, in order to facilitate the mapping of loci that may affect various traits of interest.

Genomic mismatch scanning (U.S. Pat. No. 5,376,526), referred to hereafter as GMS, is an example of a very promising, massively parallel technique for the analysis of regions of DNA sequence identity (lack of variation) in individuals who are concordant for a trait, and is especially promising for analyses of DNA from (disease-) affected-pedigree-member pairs. The final step in GMS is the hybridization of perfectly matched hetero-hybrid DNA duplexes (i.e., mismatch-free DNA duplexes with complementary strands from each of two affected-pedigree-members) to a reference panel of DNA hybridization probes spanning the genome (or portion thereof) under study. These DNA hybridization probes are obtained by recombinant DNA cloning, which again leads to the problem mentioned above concerning the physical integrity of such probes and their ability to span the entire genome under study.

U.S. Pat. No. 4,395,486 and Botstein, D., et al.; Am. J. Hum. Genet. 1980, 32, 314-331 describe the use of “restriction fragment length polymorphisms” (RFLP) for the identification of differences in the sequence of a specific region of a polynucleotide using physical samples (e.g., DNA) obtained from different individuals. They showed that if a molecular clone of a region of interest in the human genome was available, one could use restriction endonuclease digests of genomic DNA followed by Southern blotting (see Southern, E. M.; J. Mol. Biol. 1975, 98, 503-517) in an attempt to detect distinguishable fragmentation patterns that represent one or more co-dominant alleles from the region in question, and use this information for the construction of genetic linkage maps. If found, an RFLP typically arises due to a sequence variation that disrupts the recognition sequence of the restriction enzyme used for the analysis.

Later, when the PCR became widely adopted, it was realized that SSRs or “simple sequence” repeats (usually of di-, tri-, or tetra-nucleotides), which are found throughout the human genome, provide an even more amenable resource for the identification and analysis of differences in the sequence of a specific region of a polynucleotide using physical samples (e.g., DNA) obtained from different individuals (see U.S. Pat. No. 5,075,217 and Weber, J. L., et al.; Am. J. Hum. Genet. 1989, 44, 388-396). Although traditional RFLPs had facilitated many significant advances in human genetics, PCR-based analyses of SSRs proved even more useful, as simple-sequence length polymorphisms (SSLPs) generally display greater heterozygosity, can be scored without having to use radioisotopes, and the region of DNA sequence that is probed for variation is not limited to restriction-enzyme recognition sequences.

Regardless of the methods used to discover and analyze them, polynucleotide length polymorphisms (RFLPs or SSLPs) are extremely useful for the development of genetic maps, and transmission- or population-based studies of human genetic diseases. Restriction landmark genomic scanning (RLGS), described in Hatada, I., et al.; 1991, Proc. Natl. Acad. Sci. USA, 88, 9523-9527, is a scalable, two-dimensional approach for the analysis of polynucleotide length polymorphisms. RLGS involves the digestion of genomic DNA using a single restriction enzyme, end-labeling of the fragments so obtained with a radioisotope label, and the subsequent fractionation of these fragments by (i) agarose gel electrophoresis in one dimension, (ii) digestion of the electrophoresed DNA in situ using a second restriction enzyme, and (iii) subsequent polyacrylamide gel electrophoresis in a second dimension. RLGS is noteworthy because of its ability to analyze multiple loci in a manner that does not rely on cloned polynucleotides or sequencing information derived thereof. Despite its utility, RLGS is a complicated procedure that is not amenable to standardization, laboratory automation, and computational emulation; and relies on reagents (radioisotopes) that most investigators would strongly prefer to avoid.

U.S. Pat. No. 5,871,697 describes a scalable approach for the analysis of restriction fragment length polymorphisms in cDNA. This approach, called “Quantitative Expression Analysis” or QEA, includes (i) the use of restriction enzyme recognition-sequences as targets; (ii) the use of the PCR using two different “generic” primers whose synthetic annealing sites are introduced artificially by the ligation of double-stranded oligonucleotide adapters of known sequence on either end of the fragments to be amplified; and (iii) computational emulation of laboratory methods. Each QEA reaction queries a physical sample of polynucleotides for the presence or absence of only two targets (which must be restriction enzyme recognition-sequences), and consequently can only be used with cDNA. QEA cannot be used to query genomic DNA because of the method's limited information-querying capacity. U.S. Pat. No. 5,871,697 attempts to address this inadequacy with a very different technique, “Colony Calling” (CC), which is described in a second embodiment. When embodied as a laboratory method, CC relies on the use of a set of 20 polynucleotide hybridization probes in order to develop a 20-bit binary hash code to characterize the inserts found in a library of arrayed DNA clones. The CC technique cannot provide length information about the distance between a pair of detected targets, and suffers from, inter alia, all of the problems associated with the use of polynucleotide hybridization probes and recombinant DNA cloning that were mentioned earlier. The only common feature of the QEA and CC laboratory techniques is that they can be emulated computationally, and even this similarity involves the use of quite different algorithms and data structures.

The present invention meets the many needs discussed above. It provides a computational method that is flexible and efficient at comparing large amounts of biopolymer sequence data, and importantly, can be emulated in the laboratory. The disclosed laboratory procedure not only emulates the computational method, it provides a powerful laboratory procedure for comparing polynucleotide sequences as well. The present invention provides a method that allows the analysis and isolation of large numbers of specific structured query fragments of interest. Remarkably, it accomplishes this without the use of cloning techniques or polynucleotide amplification protocols that require locus-specific probes that hybridize to a sequence of interest. Furthermore, since the present invention possesses the aforementioned attributes, it provides a scalable laboratory method for identifying genomic sequences that affect certain traits.

### SUMMARY OF THE INVENTION

The disclosed invention is: (i) a versatile and powerful computational approach that efficiently analyzes sets of biopolymer sequence data of any size, including an entire genome, deriving useful information thereof; and (ii) a versatile strategy that may be implemented, on any desired scale, in a laboratory using a physical sample (or samples) of polynucleotides. This laboratory strategy is an emulation of the related computational approach, and can be used to derive useful information (e.g., about DNA sequence variation or identity) and physical products from a polynucleotide sample or samples. Because of its power and flexibility, the present invention allows entire genomes to be compared in an extremely precise manner. For example, very large numbers of DNA fragment length polymorphisms distributed throughout the human genome can be detected in individuals and mapped on a reference genome sequence using the present invention.

More specifically, the present invention is a combinatorial, hierarchical method that uses “process-patterns” in one preferred embodiment to identify, classify, and compare substrings within strings; and in another preferred embodiment to identify, classify, compare, generate, and separate fragments derived from one or more physical samples of polynucleotides. These substrings (and their physical polynucleotide counterparts) are called “partition” fragments, and their process-pattern-defined derivatives are called “structured query fragments” (SQFs).

Process-pattern search targets are derived from a small set of non-coincident search targets (a search “target-group”, see Examples Tables 3-5), where each search “target” is comprised of one or more short “target strings”, which are typically six characters long when the string(s) under study are polynucleotide sequences. A single search target, “target Qa” or simply “target A” (where A, B, C, etc. are identifiers, not literals) is used to partition the string(s) under study into substrings, producing either “Qa-Qa” or “Qa-[non-Qa]” fragments, called “partition” fragments. The “Qa-Qa,” also referred to as “A-A,” partition fragments so obtained are then typically queried using the remaining members of the target-group, which are organized into a small number of “major” classes (e.g., classes B, C, D, E, and F). Each major class is a ranked set of a limited number of members (e.g., B1, B2, B3, and B4 in class B).

The number of “major classes” in a search target-group determines the number of search steps required to define a process-pattern. Each search step: (i) is specific for a given major class, where the major class for each step is selected without replacement from the major classes that define is the search target-group; (ii) proceeds in a specific direction over a process-defined, restricted region of the partition fragment; (ii) seeks the highest-ranked member of the current search class in the current search region; (iii) if successful, truncates the current search region and limits the search region for the next search step; (iv) is part of a process that defines a pattern, where for a given target-group, each site in the pattern indicates the presence of the site found—and the absence of higher-ranked members of the same class—in that site's process-defined search region.

Combinatorics is an important feature of certain preferred embodiments of the current invention. In these embodiments, the order of major search classes used to define process-patterns is permuted (e.g., [B, C, D, E, F] vs. [C, B, D, E, F]). Each partition fragment is queried for the presence of all of the process-patterns that can be generated using all of the possible permutations of the major classes in the search target-group, and using both of the possible starting directions for the first search step. Thus, a well-designed search target-group comprised of a limited number of small search targets can query a genome at very high frequency.

A structured query fragment is simply a fragment bounded by two sites in a process-pattern. Typically, two SQFs adjacent to the search target site detected in the final search step are of most interest.

In the computational preferred embodiment, the computer software disclosed herein locates the process-patterns and SQFs within the partition fragments in the string(s) under study (e.g., a set of polynucleotide sequence data), stores the results, and provides for access to this data by database query and analysis software. These computational analyses are emulated by the laboratory-preferred embodiment, which uses physical samples of polynucleotides and the laboratory methods disclosed herein. In the latter, cleavage effectors, including restriction endonucleases and any other equivalent sequence-specific endonuclease, cleavage reagent or process, preferably restriction endonucleases, utilize as substrates and generate as products progressively expanding sets of asymmetrically end-immobilized DNA, a process that ultimately yields extremely large numbers of individually distinguishable SQFs (called “ranged” SQFs) with lengths typically between 100-700 nucleotides. The known process-pattern and observed length of an experimentally obtained ranged SQF typically provide sufficient information for the computer software disclosed herein to map the ranged SQF automatically to its partition fragment (and location) within a set of polynucleotide sequence data that characterizes the physical sample(s) of polynucleotides under study.

The laboratory preferred embodiment of the disclosed invention addresses the limitations associated with the use of cloning or information derived thereof to obtain polynucleotide hybridization probes. This embodiment does not use cloning but can generate from genomic DNA extremely large numbers of individual structured query fragments (SQFs) or pools thereof whose partially characterized sequence properties allow them to be mapped automatically using the computational preferred embodiment of the disclosed invention. In some embodiments, these SQFs can be immobilized on solid supports (e.g., spatially addressable microarrays) and used for a variety of useful preparative and analytical procedures that previously relied on the use of polynucleotide hybridization probes obtained directly by recombinant DNA cloning or as synthetic oligonucleotides whose sequence was determined from a polynucleotide fragment obtained by recombinant DNA cloning. Some important examples of procedures involving the use of SQFs as hybridization probes include the identification and mapping of RNA transcripts, gene discovery, and quantitative analyses of gene expression.

The disclosed invention has an essentially unlimited potential to address the needs discussed earlier because of the essentially unlimited flexibility it affords the investigator in the selection of the members of a search target-group and the definition of process-patterns, and because of the lack of reliance on cloning techniques and locus-specific amplification of polynucleotide regions of interest.

### BRIEF DESCRIPTION OF THE DRAWINGS

FIG. 1 is a general summary of the workflow for various aspects of the present invention.

FIGS. 2 (A), (B), (C), and (D) show the table schema of the relational database application developed for a preferred embodiment of the present invention.

FIGS. 3 (A), (B), (C), (D), (E), (F), and (G) show various general features of a software program developed using the Enterprise Edition of Microsoft® Visual Basic™ 6.0 (Service Pack 3) using the software object references (FIG. 3F) and compiler instructions (FIG. 3G) shown, where the software program typically uses the indicated form and related objects and files (FIGS. 3A, 3D, and 3E), and file-system directories (FIGS. 3B and 3C), as a user-interface to obtain specifications for the automated acquisition and input of polynucleotide sequence data into the relational database application developed for a preferred embodiment of the present invention.

FIGS. 4 (A), (B), (C), (D), (E), (F), (G), (H), (I), (J), and (K) represent a flowchart that describes the execution of the software program introduced in FIG. 3 and that is typically used for the automated acquisition and input of polynucleotide sequence data into the relational database application developed for a preferred embodiment of the present invention.

FIGS. 5 (A), (B), (C), (D), (E), (F), (G), and (H) represent a flowchart that describes the typical execution of a Transact-SQL stored procedure (FIGS. 5A, 5B, 5C, and 5D) used to “register” newly acquired polynucleotide sequences, a process that typically involves scoring the occurrence therein of all of the “registered” search targets (more specifically, their search target-strings) in the relational database application developed for a preferred embodiment of the present invention; and another Transact-SQL stored procedure (FIGS. 5E, 5F, 5G, and 5H) that is used to “register” search targets, a process that typically involves scoring the occurrence of newly designed search targets (more specifically, their search target-strings) in all of the “registered” polynucleotide sequences in the relational database application developed for a preferred embodiment of the present invention.

FIGS. 6 (A), (B), (C), (D), (E), (F), (G), and (H) represent a flowchart that describes the execution of Transact-SQL stored procedures typically used by a database administrator to provide a batch-processing service that executes newly designed SQF analyses and updates existing SQF analyses, where the flowchart in these figures shows the execution of SQF analyses up to the level of processing “all-classes-present” fragments (see FIG. 7), and where the said stored procedures are part of, and are executed in, the relational database application developed for a preferred embodiment of the present invention.

FIGS. 7 (A), (B), (C), (D), (E), (F), (G), (H), (I), (J), and (K) represent a flowchart that describes Transact-SQL stored procedures typically used to execute newly designed SQF analyses and/or update existing SQF analyses, where the flowchart in these figures shows the execution of an SQF analysis at the level of searching an “all-classes-present” fragment for the presence of all of the process-patterns and SQFs of interest that may be present therein, and where the said stored procedures are part of, and are executed in, the relational database application developed for a preferred embodiment of the present invention.

FIGS. 8 (A), (B), (C), (D), (E), (F), and (G) represent a flowchart that illustrates how a relatively simple 5×4 search target-group may be used to generate a very large number of process-patterns and SQFs of interest, where this illustration of “comprehensive-scale” processing of a polynucleotide sample considers all of the 120 class-order permutations that can typically be generated from a 5×4 search target-group in a preferred embodiment of the present invention.

FIG. 9 schematically illustrates how a relatively simple 5×4 search target-group may be used to generate a very large number of process-patterns and SQFs, where this illustration of “variable-scale” processing of a polynucleotide sample only shows 2 of the 1024 process-patterns and 4 of the 2048 SQF-fractions obtained from only one (“BCDEF”) of the 120 class-order permutations that can typically be generated from a 5×4 search target-group in a preferred embodiment of the present invention.

FIGS. 10 (A), (B), (C), (D), (E), and (F) schematically illustrate salient features of a preferred laboratory embodiment of the present invention, where this illustration of “variable-scale” processing of a polynucleotide sample considers only one (“BCDEF”) of the 120 class-order permutations that can typically be generated from a 5×4 search target-group in a preferred embodiment of the present invention.

FIGS. 11 (A), (B), (C), (D), and (E) schematically illustrate salient features of a preferred laboratory embodiment of the present invention, where a 5×4 search target-group is used for a version of SQF analysis for the detection of SQFs that are identical-by-descent (i.e., lack any sequence variation), typically in DNA samples obtained from two related individuals who are members of an affected-pedigree-member (“APM”) pair.

FIGS. 12 (A), (B), (C), (D), (E), and (F) represent a flowchart that describes the execution of a Transact-SQL stored procedure typically used to execute simulated SQF analyses, where the said stored procedure is part of, and is executed in, the relational database application developed for a preferred embodiment of the present invention.

### DESCRIPTION OF THE PREFERRED EMBODIMENTS

I. Methods for characterizing a set of strings.

In one aspect, the present invention provides a method for characterizing qualifying substrings present within a set of strings. The method comprises:

a) receiving the set of strings;

b) defining individual search targets, and assembling a limited number of the search targets into one or more search target groups that may be used to generate process-patterns and SQFs from qualifying substrings present within the set of strings; and

c) processing each qualifying substring from the set of strings through all of the possible variations of an ordered series of search steps, where the number of steps in each ordered series of search steps is equal to the number of major search classes, as defined herein, in the search target-group. Each search step is specific for one of the major search classes, and involves the attempted discovery of an appropriate search target-site, and the subsequent delimitation of the search region for the next step if the appropriate search target-site is discovered. Each of the possible ordered series of search steps of the procedure may result in the detection of a process-dependent pattern of search target sites. Any process-patterns so obtained, or “structured query fragments” (SQFs) definable therein by two search target sites, may be used to characterize the qualifying substrings present within the set of strings, and compare the process-pattern containing substrings so characterized with substrings derived from any other set of strings that have also been subjected to SQF analysis using the same search target-group. Such comparisons are based on the presence of one or more of the same specific process-patterns that are found in both or all of the compared substrings using the same search target group.

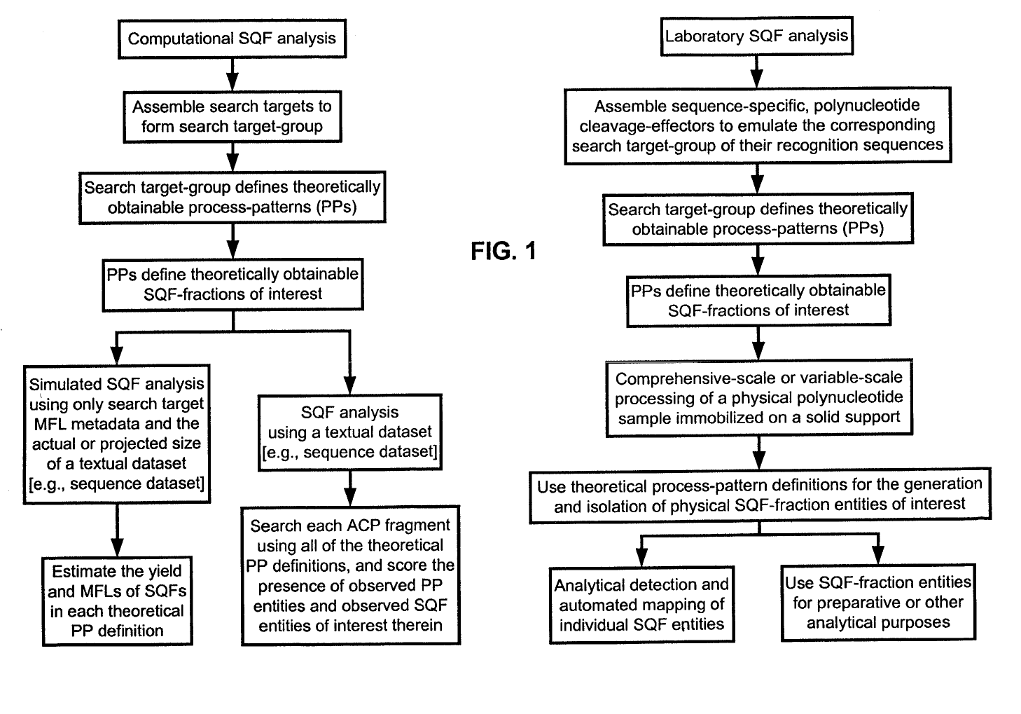

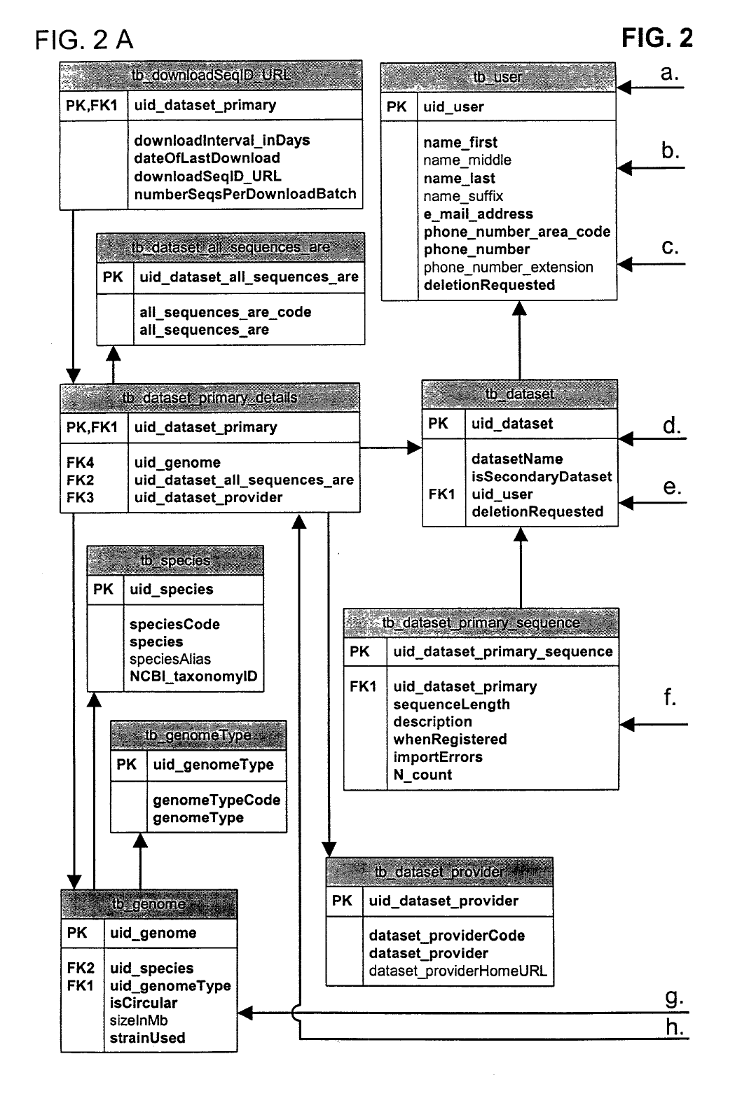

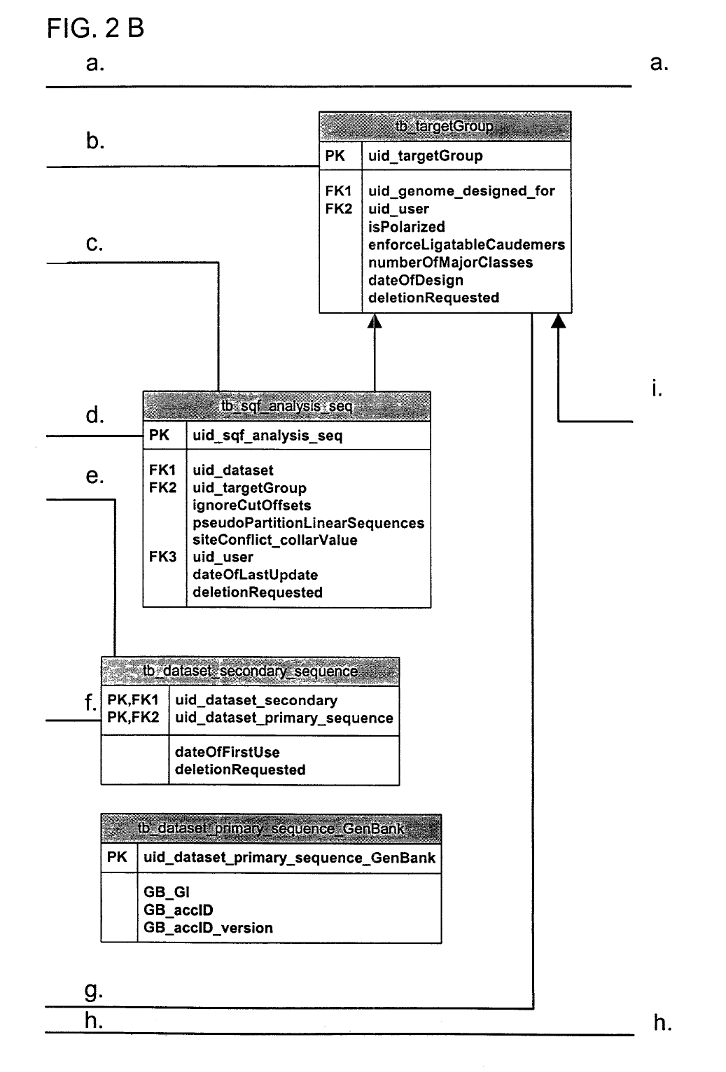

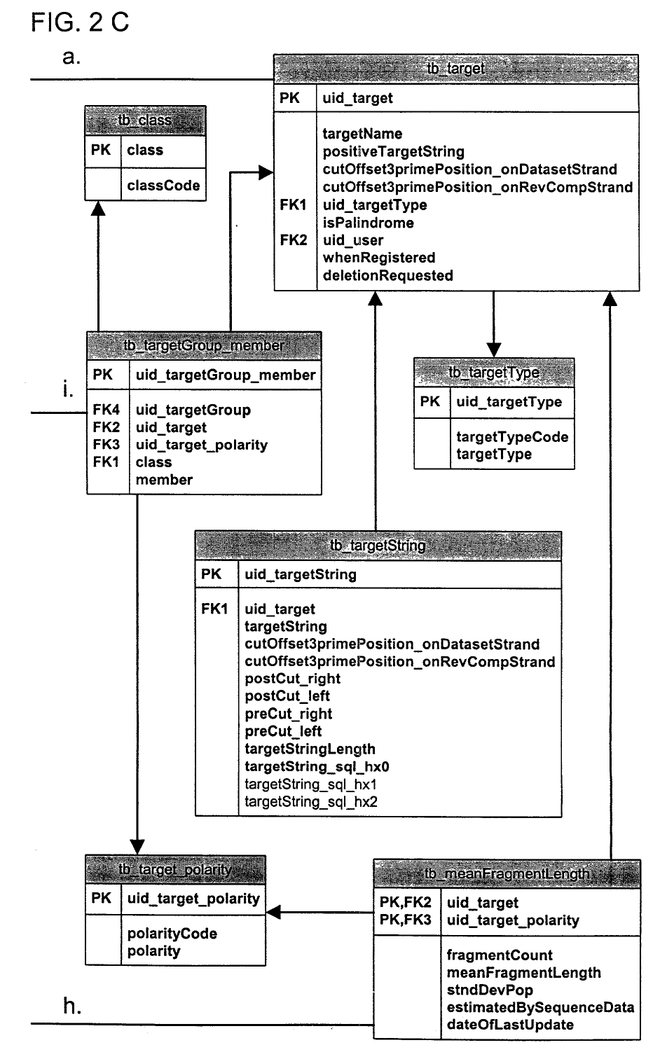

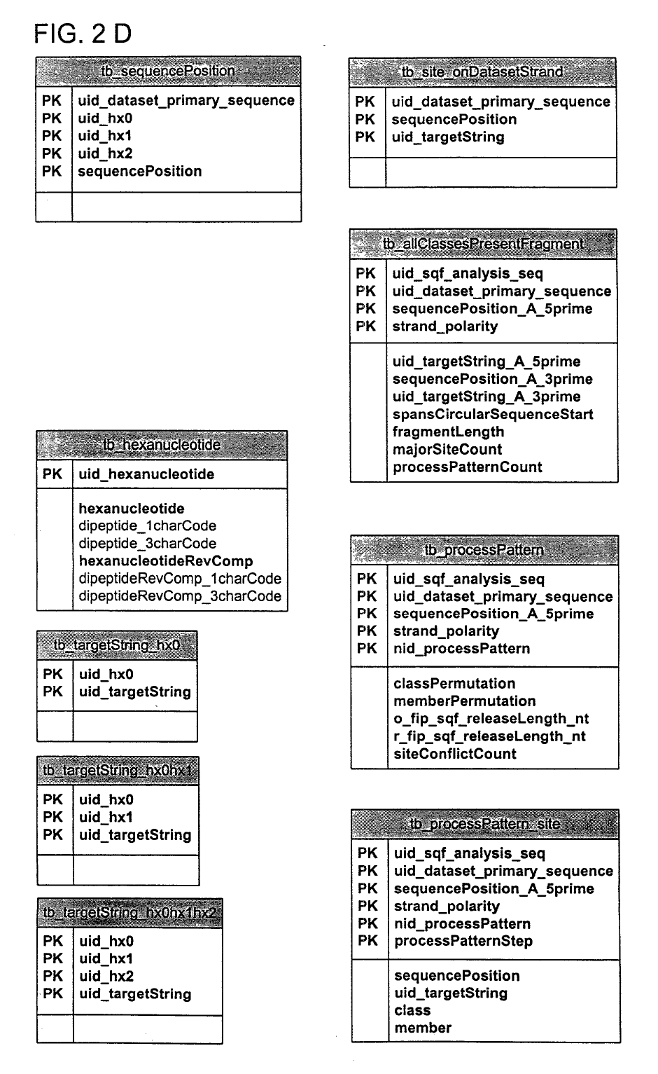

The present invention (FIG. 1) involves laboratory procedures, described in detail later in the specification, and bioinformatic procedures. The bioinformatic procedures are computational methods, including computer program code, algorithms, data structures and the like, typically for a relational database application (FIG. 2). One of these computational methods defines, locates, and stores the results of one or more searches within one or more sets of strings for “process-patterns” and their SQF derivatives. A structured query fragment is simply a fragment bounded by two sites in a process-pattern. In practice, two SQFs adjacent to the site detected in the final search step are of most interest.

To summarize, a process-pattern is both: (i) an ordered set of short “targets” (one from each major search class) that must be present, and whose higher-ranked members of the same major search class must be absent, within the relevant search area of a partition fragment, and (ii) a step-wise delimitation process (where each step has a defined polarity and occurs after a search target is found) that restricts the region of a partition fragment where the next major-class-specific, pre-emptive target-search takes place (Examples shown in Tables 14 and 15).

A) Receiving the set of strings.

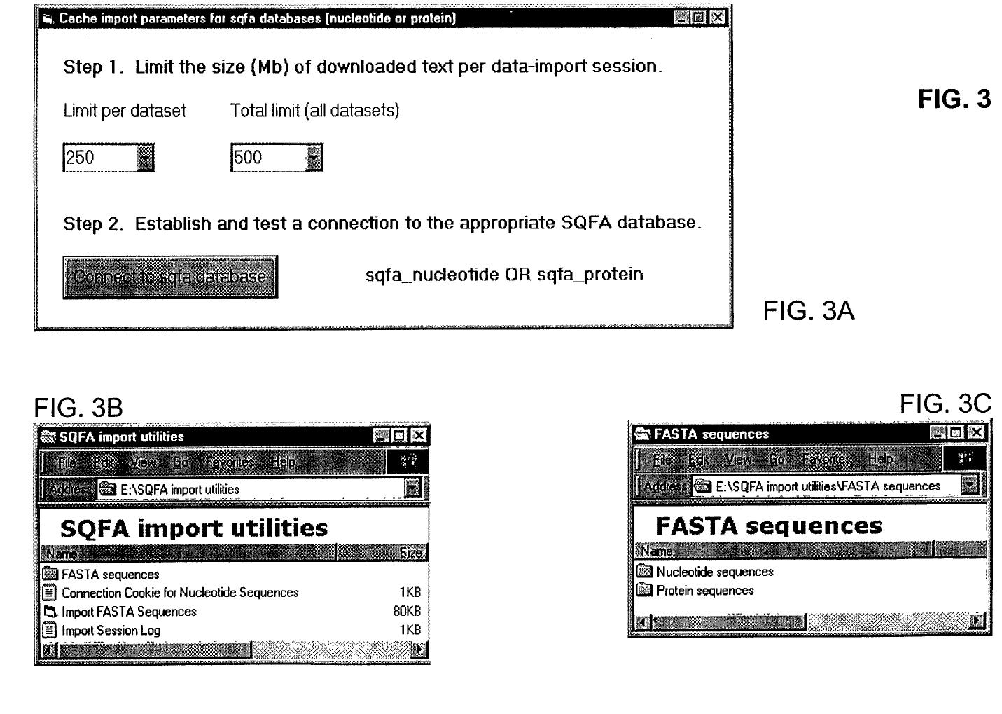

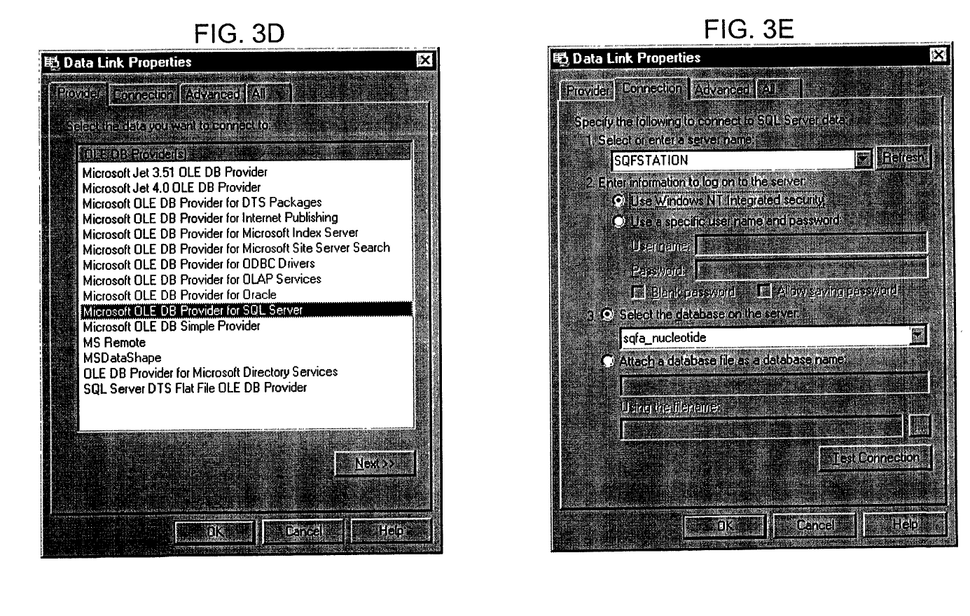

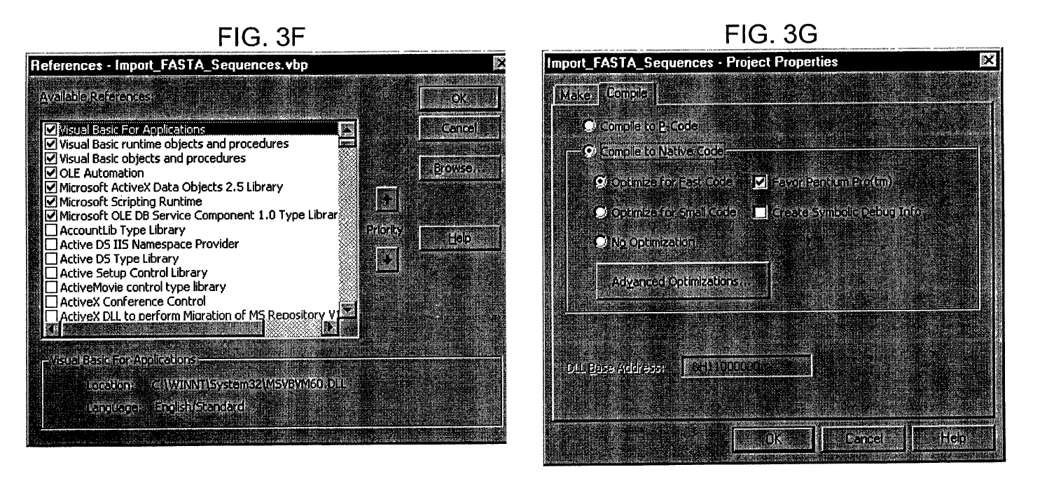

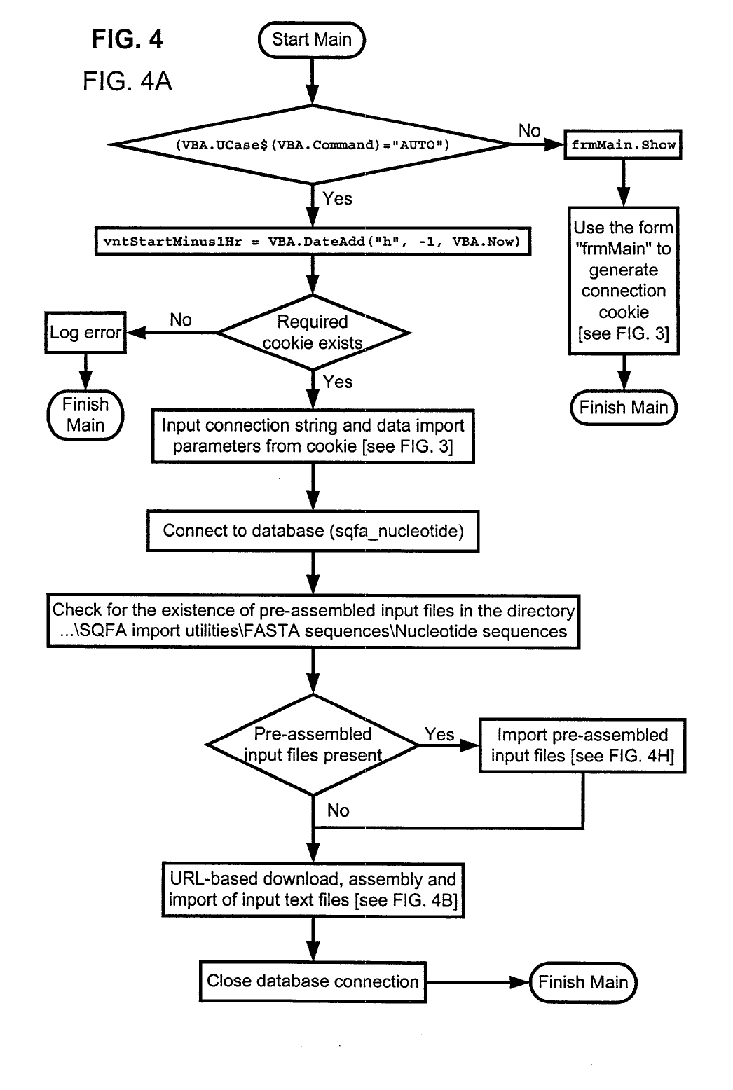

Typically for the bioinformatic procedures of the present invention, the set of strings are received as a computer file, for example, but not limited to, a text file of FASTA-formatted polynucleotide sequences. Upon receipt, the set of strings is typically stored in a database application, preferably a relational database application, as described herein for a preferred embodiment of the present invention (FIG. 2). The database application may be developed and implemented using commercially available database management systems. The database management system of the current invention typically includes database software, preferably client-server or equivalent database software for example, but not intended to be limited to, Microsoft® SQL Server, Oracle®, or IBM® DB2™. The database software runs on a computer operating system, preferably a server operating system such as for example, but not intended to be limiting, Unix®, Linux, or Microsoft® Windows® NT™ or Windows® 2000. In one preferred embodiment, the database application is developed, implemented, and maintained using Microsoft® SQL Server 7.0 (Service Pack 1), running on an Intel®-processor-based personal computer running Microsoft® Windows® NT™ Server version 4.0 (Service Pack 6) as a computer operating system (FIG. 2). The data-import software application (FIG. 3) of the current invention may be developed using a software development environment or programming language, of which many appropriate examples, such as, but not limited to, Delphi®), PowerBuilder®, Microsoft® Visual Basic™, or Microsoft® Visual C++, which are well known in the art. In one preferred embodiment, the data-import software application (FIG. 3) is developed using the Enterprise Edition of Microsoft® Visual Basic™6.0 (Service Pack 3).

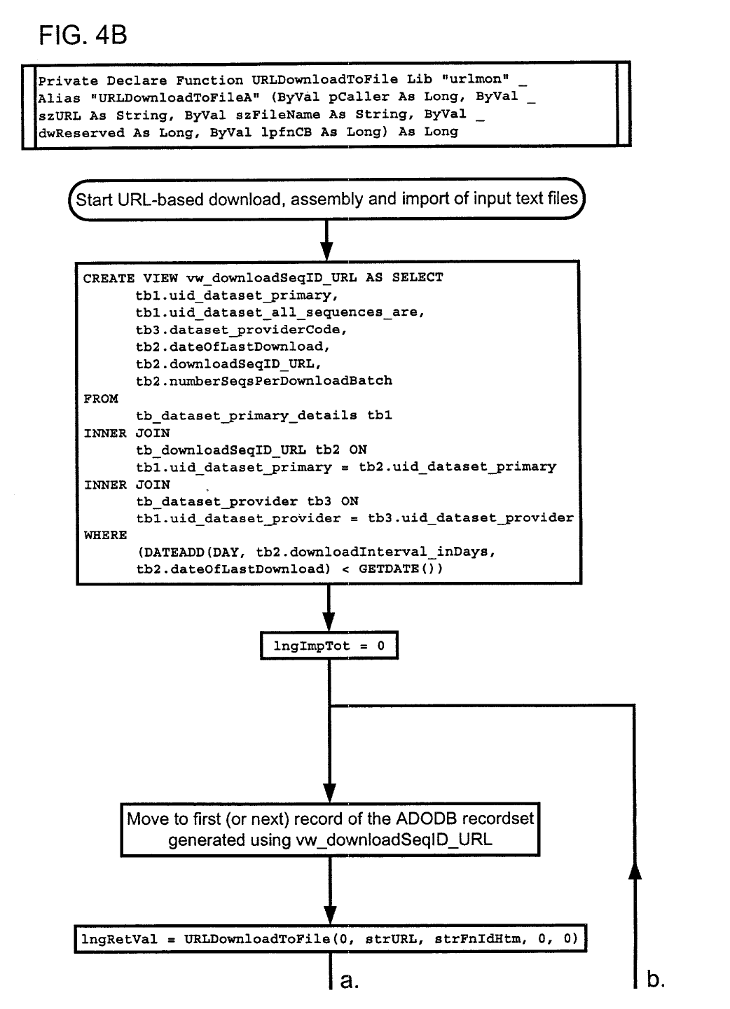

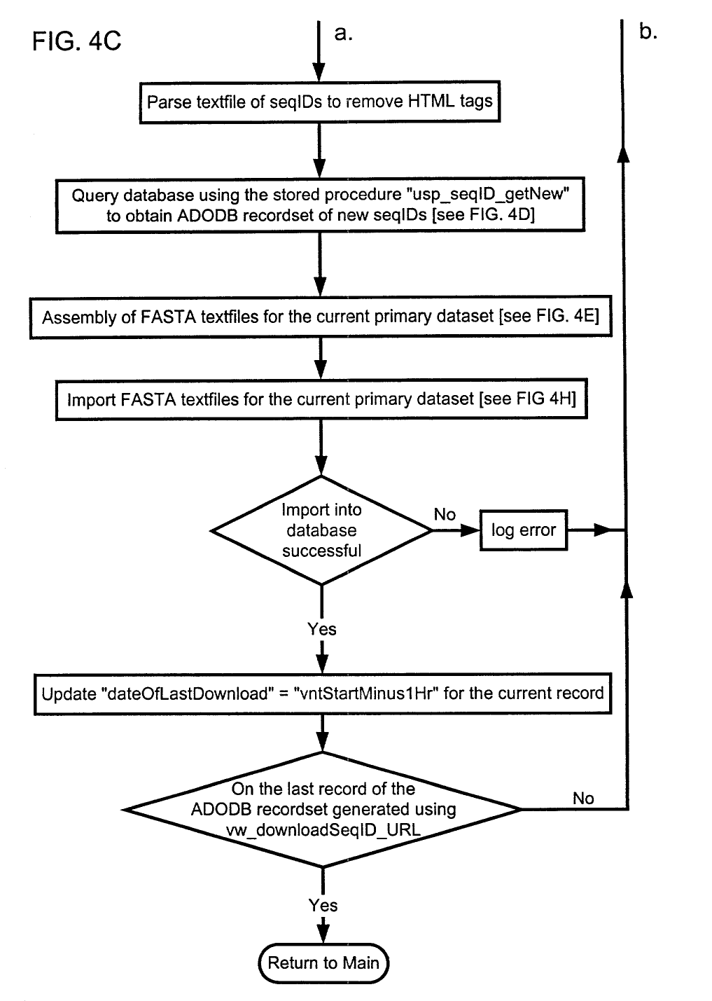

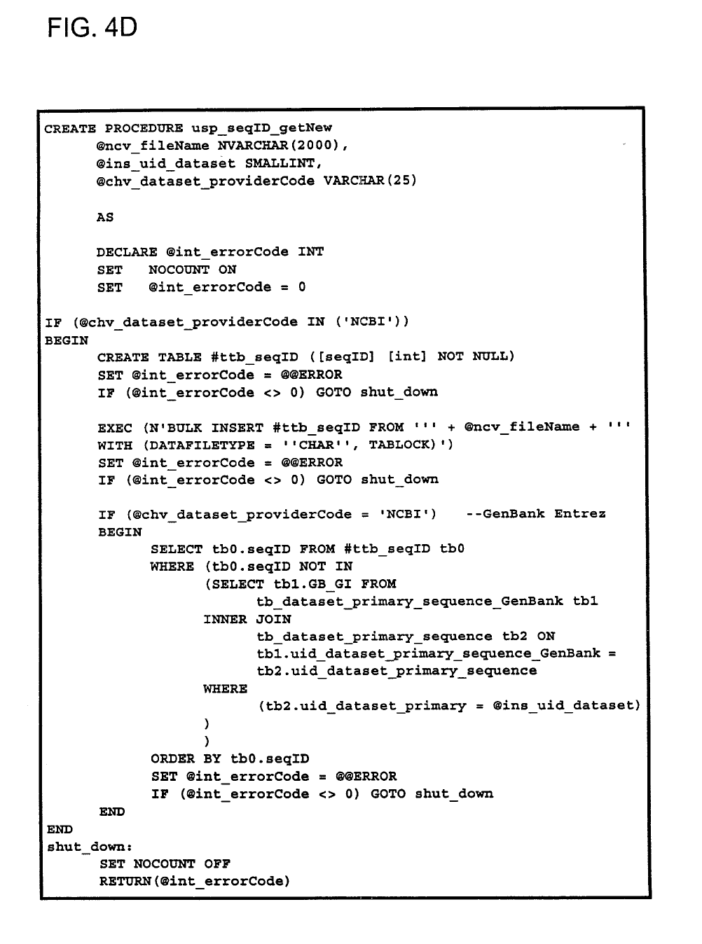

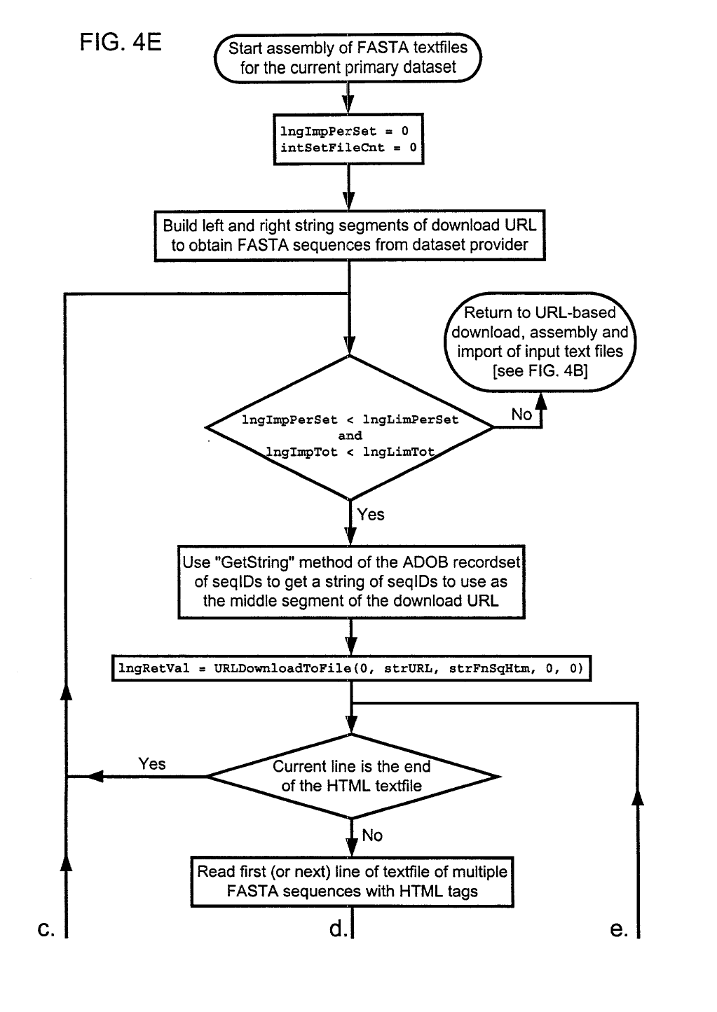

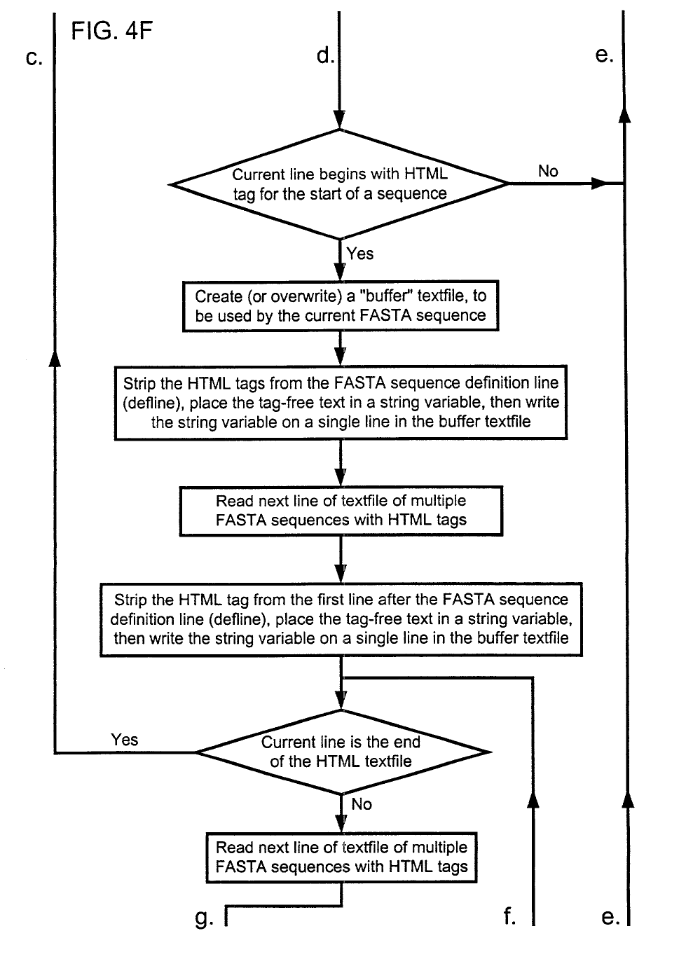

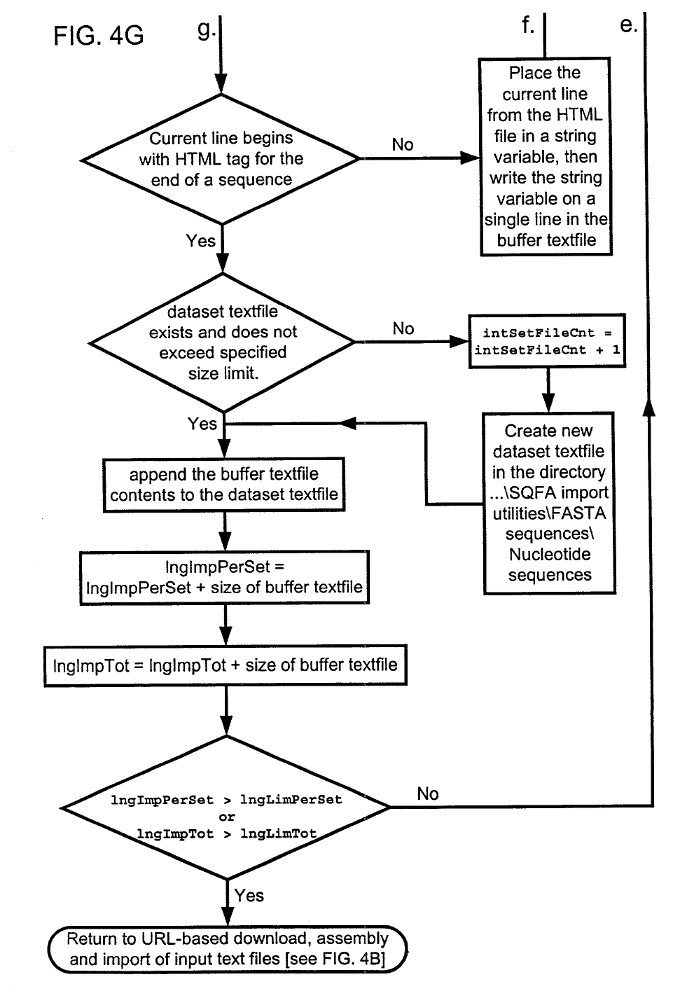

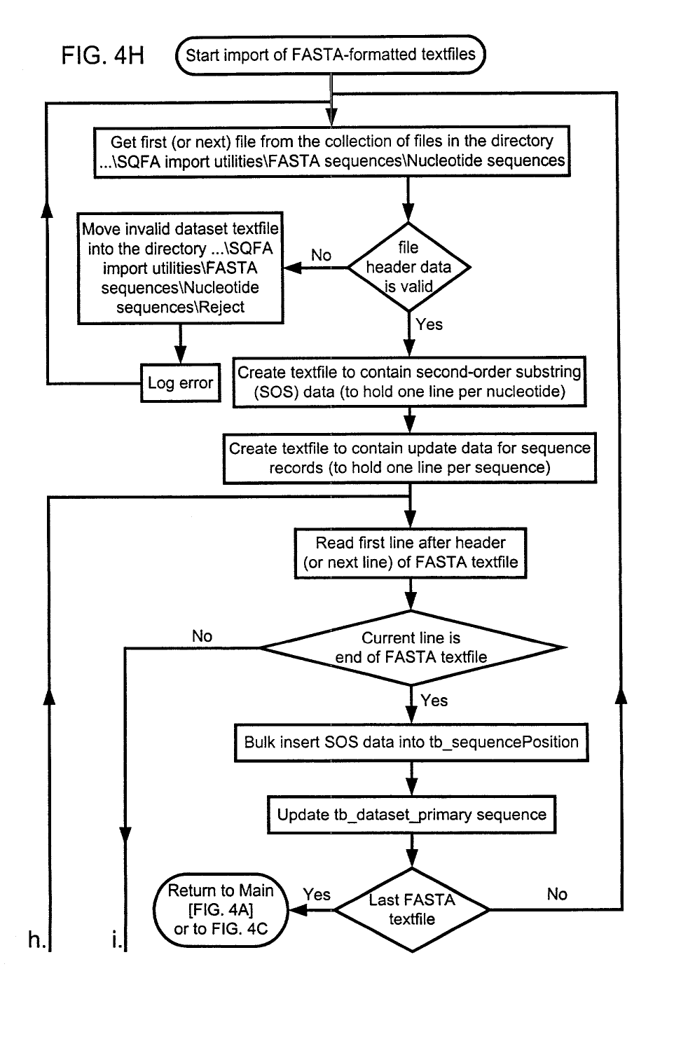

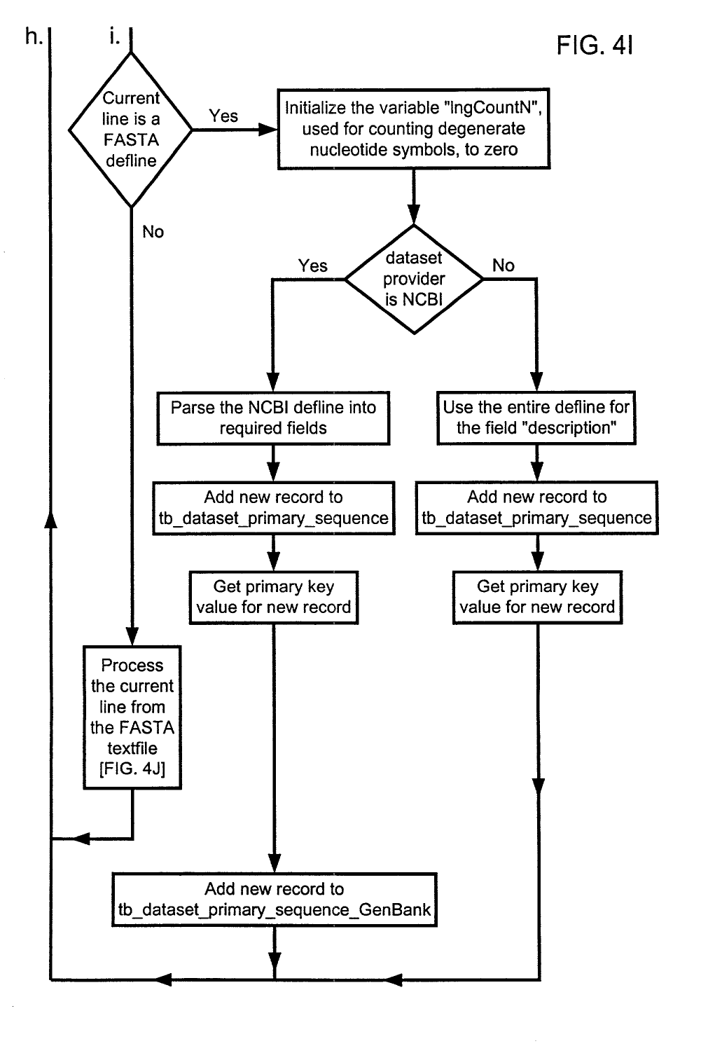

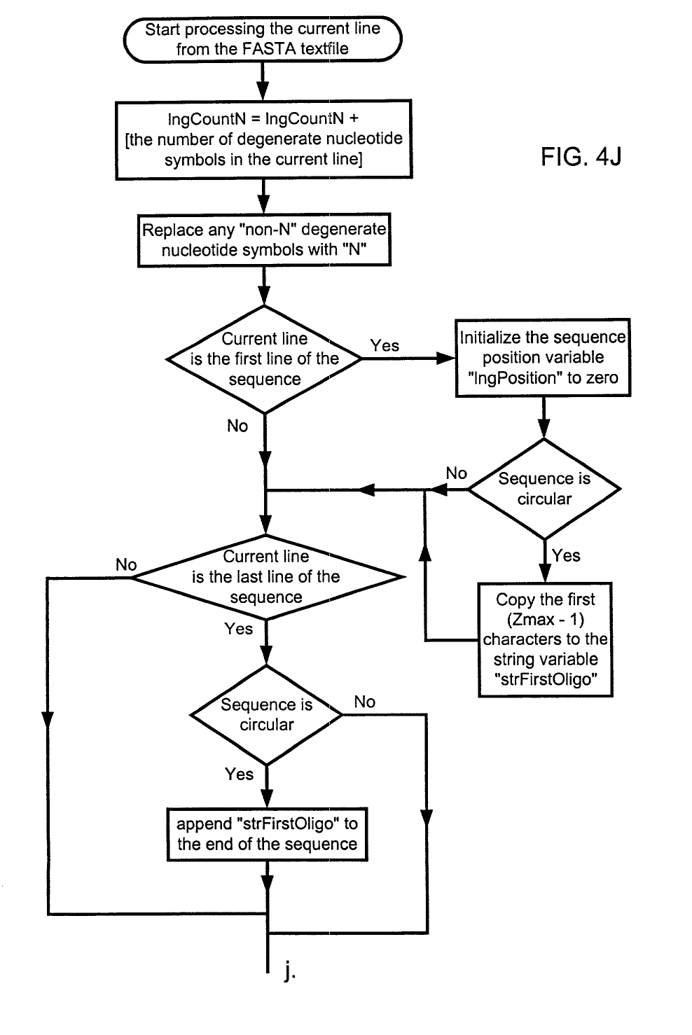

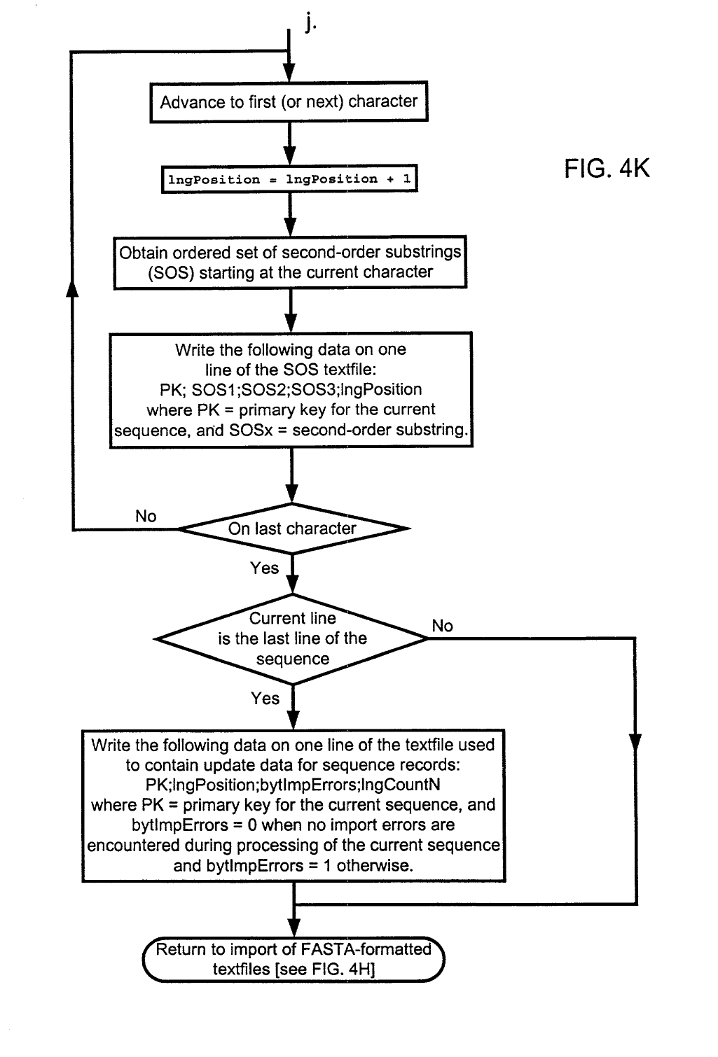

Relevant references that describe the use of Microsoft® Windows® NT™ Server version 4.0, Microsoft(E) SQL Server 7.0, the Transact-SQL programming language used for programming SQL Server database applications, ActiveX® Data Objects (ADO, an object-interface that facilitates interaction between the relational database and the data-import software application), and the Microsoft® Visual Basic™ 6.0 software programming language include the following: (i) Sussman, D. (1999) ADO 2.1 Programmer's Reference; Wrox Press: Birmingham, U.K.; (ii) Microsoft Corporation (1999) SQL Server 7.0 Books Online; Microsoft Press: Redmond, Wash.; (iii) Soukup, R. and Delaney, K. (1999) Inside Microsoft SQL Server 7.0; Microsoft Press: Redmond, Wash.; (iv) DeLuca, S. A. et al. (2000) Microsoft SQL Server 7.0 Performance Tuning Technical Reference; Microsoft Press: Redmond, Wash.; Amo, W. C. (1998) Transact-SQL; IDG Books Worldwide, Foster City Calif.; (v) Microsoft Corporation (1998) Microsoft Visual Basic 6.0 Programmer's Guide; Microsoft Press: Redmond, Wash.; and (vi) Minasi, M. (1999) Mastering Windows NT Server 4, 6^{th} ed.; Network Press, San Francisco, Calif. The function “URLDownloadToFile” (FIG. 4B), a critically important function used by the data-import program, is described in the Microsoft® Knowledge Base (Article ID Q244757), which is available at the Microsoft® Corporation's website (http://www.microsoft.com). The publicly accessible National Center for Biotechnology Information (NCBI) web-application utilities “PmQty” and “Retrieve” (see Examples Table 2) used by the data-import program are described at the following NCBI websites: (http://www.ncbi.nim.nih.gov/entrez/utils/pmqty_help.html) and (http://www.ncbi. nim.nih.gov/entrez/query/static/linking. html), respectively.

1) Primary Datasets.

a.) General Considerations.

Typically the set of strings that is received is a primary dataset. Primary datasets (Dp) typically represent a set of strings where each string is unique in a primary dataset and may not be present in any other primary dataset. Furthermore, each primary dataset is comprised of one or more strings that are all of the same type, and may be obtained from an external source and imported into the relational database application. As a non-limiting example, the external source may be a public biopolymer sequence database, such as the databases available at the NCBI. As another non-limiting example, the external source may be a private biopolymer sequence database.

Preferably, for a given embodiment of the present invention, each of the strings present in the relational database application represents the primary sequence of one general type of biopolymer. For example, in one embodiment, each string in the database may represent the amino acid sequence of a protein or polypeptide or equivalent polymer; whereas in another embodiment, each string in a separate database may represent a polynucleotide sequence. Some of the strings in a given database used for a particular embodiment of the present invention may be linear strings of characters that are linear entities, and are treated as such for all aspects of the invention; whereas other strings in the same database may be linear strings of characters that actually represent circular strings of characters, and thus are treated as circular entities.

In certain embodiments, each string of characters in the primary dataset represents the sequence of a linear or circular polynucleotide (see Examples Table 1). The polynucleotide sequences are typically described using the standard conventions and nucleotide monomer symbols that are commonly understood in the relevant scientific literature. In these embodiments involving polynucleotide sequences, some or all of the primary datasets may represent sets of deoxyribonucleic acid (DNA) sequences from the following sources: a nuclear genome, mitochondrial genome, chloroplast genome, viral or other microbial genome, episome, plasmid, molecular clone, sequencing contig, sequencing fragment, or any other source or type of DNA molecule or the like.

Some or all of the primary datasets may also represent: putative or hypothetical polynucleotide sequences, such as reverse translations of protein sequences; complementary DNA (cDNA) sequences; or ribonucleic acid (RNA) sequences that are stored as the equivalent DNA sequences.

In certain embodiments, the input-string representing each polynucleotide sequence is referred to as the “dataset strand”, and its reverse-complementary strand is referred to as the “reverse-complement strand” for the sequence in question. For certain embodiments, the only valid characters that may appear in the inputted polynucleotide sequences are either members of the relevant set of non-degenerate nucleotide symbols, or the fully degenerate nucleotide symbol, N; and where N is assumed to be analogous to an unknown, or yet-to-be-determined, or null value. Typically, N is explicitly forbidden to denote that all non-degenerate variants of N are known to occur at a sequence position where N occurs.

b. Coding Table for Processing a Set of Strings.

In a preferred embodiment, the primary dataset is created by first inputting strings belonging to primary datasets into a relational database application and storing each inputted string (input-string) of variable length (L) characters as an ordered set of (L) database records, where each record includes (n) ordered non-nullable data fields, with each such data field containing a signed integer value identifying a certain number of characters surrounding, preceding, or following a character at a particular position in the inputted string.

One detailed approach for creating the primary datasets described above includes the following (as described in FIG. 4; see also Examples Tables 8-10):

(i) acquiring, in a programmatically scheduled or unscheduled manner; assembling; and preparing appropriately formatted computer text files required for Steps (ii to vi) following, where each text file contains one or more input-strings, and where all such strings in a given text file are all members of the same primary dataset (Dpₓ), and where the first few lines of text in each computer text file typically contain information that identifies the primary dataset (Dpₓ) and some of its properties (FIG. 4);

(ii) obtaining from each input-string of variable length (L) characters an ordered set of (L) first-order substrings of maximum length (z=zₘₐₓ), where the first-order substrings start at each character position (p) (where p=1, 2, . . . , L) of each input-string; and where for a linear input-string of characters representing a circular string of characters, a special “wrapping” substring of length (zₘₐₓ−1) and obtained from position (p)=1 to position (p)={zₘₐₓ−1} is appended to the end of the input-string, and where for the said input-string now of apparent length (L+zₘₐₓ−1), the first-order substrings are all of constant length (z=zₘₐₓ) and contain the characters from position (p) to position (p+zₘₐₓ−1) inclusive; and for linear input-strings representing a linear string of characters the said first-order substrings are of constant length (z=zₘₐₓ) and contain the characters from position (p) to position (p+zₘₐₓ−1) inclusive where ([p+zₘₐₓ−1]≦L), and are of variable lengths (z) (where 1≦z<zₘₐₓ) from positions (p) to position L where ([p+zₘₐₓ−1] >L), as shown in Examples Tables 8-10;

(iii) obtaining from each of the ordered first-order substrings an ordered set of (n) second-order substrings of lengths (y₁, y₂, . . . , yₙ), each with a defined maximum possible length (yₘₐₓ) (where 0≦yᵢ≦yₘₐₓ and z=y₁+y₂+. . .+yₙ), starting at positions (wᵢ) (where wᵢ=[(i−1)*yₘₐₓ]+1, and i=1, 2, . . . , n) in each first-order substring, the said second-order substrings being of constant length (yᵢ=yₘₐₓ) and containing the characters from position ([(i−1)*yₘₐₓ]+1) to position (i*yₘₐₓ) inclusive where (i≦[z/yₘₐₓ]), being of variable lengths (yᵢ) and containing the characters from position ([(i−1)*yₘₐₓ]+1) to position (z) inclusive where ([z/yₘₐₓ]<i<{[z/yₘₐₓ]+1}), and being of zero length (empty string) where ({ [z/yₘₐₓ]+1} ≦i≦n) as shown in Examples Tables 8-10;

(iv) creating a database table (H) in the relational database application (Examples Table 7), where (H) contains a structured coding system for all of the possible second-order substrings of length (yⱼ), each with a defined maximum possible length (yₘₐₓ) (where 0≦yⱼ≦yₘₐₓ), that can be created using the set of characters that can be used to construct the said substrings and the parent input strings from which they are derived, where each possible second-order substring is represented as a signed integer value that functions as a unique identifier or primary key in the record for the second-order substring in the table (H); and where each such record includes a data field for the unique second-order substring that is encoded by the record's primary key, and where each of the possible signed integer values used as primary keys in table (H) may have one or more mathematical relationships with other signed integer values used as primary keys in table (H), and where each of these possible mathematical relationships may represent a structural relationship between the second-order substrings encoded by the mathematically related signed integers;

(v) creating, in a programmatically scheduled or unscheduled manner, a temporary table (#T) in the relational database application, and subsequently importing, in a programmatically scheduled or unscheduled manner, data from appropriately formatted computer text files into the temporary table (#T), where each line of text in each computer text file includes delimited data fields that include one non-nullable data field containing a unique identifier for the input-string; an ordered set of (n) non-nullable data fields containing the ordered set of (n) second-order substrings generated as described above, with one second-order substring per data field; and a non-nullable data field containing the position (p) in the input-string from which the ordered set of second-order substrings was obtained; and

(vi) using computer program code, including but not limited to Structured Query Language (SQL) commands, and the table (H) mentioned in Step (iv) above, to import the second-order substring data from the said temporary table (#T) and store the said data in an encoded form permanently in a table (7) in the relational database application, where each input-string of variable length (L) characters is stored as an ordered set of (L) database records; where each record in the said table (T) includes: one non-nullable data field containing a unique identifier for the input-string; (n) ordered non-nullable data fields, with each such data field containing a signed integer value that encodes one second-order substring of the ordered set of second-order substrings that were obtained from each position (p) in each input-string; and a non-nullable data field containing the position (p) in the input-string from which the ordered set of second-order substrings was obtained.

In certain embodiments the database table (H), also called the coding table herein, used to encode all possible second-order substrings in the said relational database application exhibits the following features and contains the following mathematical and structural relationships:

(i) the value of (yₘₐₓ) is any multiple of three and the value of (zₘₐₓ) is any desired multiple of (yₘₐₓ);

(ii) the primary key value for the second-order substring of length zero (the empty string) is 0;

(iii) when the value of (yₘₐₓ) is 6, and when the only valid characters that may appear in the inputted polynucleotide sequences are A, C, G, T, and N, then the four palindromic, non-degenerate dinucleotide second-order substrings are assigned primary key values of 21-24; the sixteen palindromic, non-degenerate tetranucleotide second-order substrings are assigned primary key values of 41-56; and the sixty-four palindromic, non-degenerate hexanucleotide second-order substrings are assigned primary key values of 61-124;

(iv) when the value of (yₘₐₓ) is 6, and when the only valid characters that may appear in the inputted polynucleotide sequences are A, C, G, T, and N, the non-palindromic, non-degenerate second-order substrings are first sorted in ascending alphabetical order for each length bracket (K) (1≦K≦6), and then for each length bracket (K) the first half of the said substrings that are not the reverse complements of each other are assigned ascending consecutive primary key values beginning at ([K*1000]+1); for each length bracket (K) the remaining half of the said substrings are assigned descending consecutive primary key values beginning at (−{[K*1000]+1}); and where second-order substrings that are the reverse complements of each other have primary key values of opposite sign but the same absolute value; and

(v) when the value of (yₘₐₓ) is 6, and when the only valid characters that may appear in the inputted polynucleotide sequences are A, C, G, T, and N, degenerate second-order substrings are sorted in ascending alphabetical order within each length bracket (K) (1≦K≦6), and then are assigned ascending consecutive primary key values beginning at 10,0001 using the length-bracket order (K)=6, 1, 2, 3, 4,and 5.

In certain embodiments of the present invention, primary datasets are stored and analyzed without the use of the coding table described earlier. For example, in one of these embodiments, all of the primary datasets in the relational database application may be comprised of strings of characters where each string represents a number. In these embodiments, typically each number is analyzed as a character string to determine the existence of process-patterns and SQFs therein. The results so obtained may be used in mathematical analyses of the numbers represented as strings in the relational database application.

In another embodiment where no coding table is required, each string of characters represents the sequence of a linear or circular protein or polypeptide, or an equivalent synthetic polymer. For such embodiments, the protein, polypeptide, or equivalent sequences are typically described using the standard conventions and amino acid or equivalent monomer symbols that are commonly understood in the relevant scientific literature.

2. Secondary datasets.

In certain preferred embodiments, the method allows individual database users to define a secondary dataset (Ds) and stores information pertaining to the secondary dataset in the relational database application (FIG. 2). For a relational database application developed for a given embodiment of the present invention, secondary datasets are typically comprised of one or more strings that may be obtained from any of the primary datasets that exist in the same relational database application. Thus in a preferred embodiment where the database application only contains polynucleotide sequence data, the strings in a secondary dataset may be of different polynucleotide types (e.g., a secondary dataset may contain a linear chromosome from the genome of one species, and a circular mitochondrial sequence from the genome of another species, and a linear fragment from a gene in the genome of a third species, and a cDNA sequence from a fourth species). Furthermore, for a given database application, the same string may be present in more than one secondary dataset. Secondary datasets are a database design feature to allow individual database users to determine rapidly the specific process-patterns and SQFs that are present in, or common to, a gene or gene family of interest using existing search target-groups that are present in the database, or if these results are unsatisfactory, to facilitate the design of new search target-groups that yield specific process-patterns and SQFs that are present in, or common to, the gene or gene family of interest.

B) Identifying a series of search target process-patterns

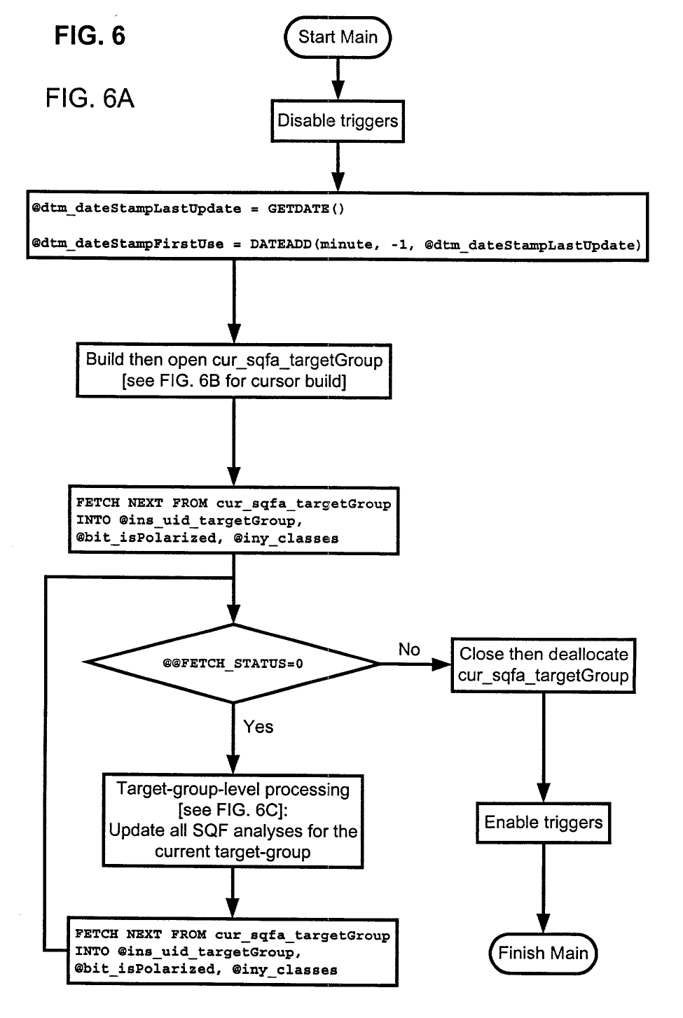

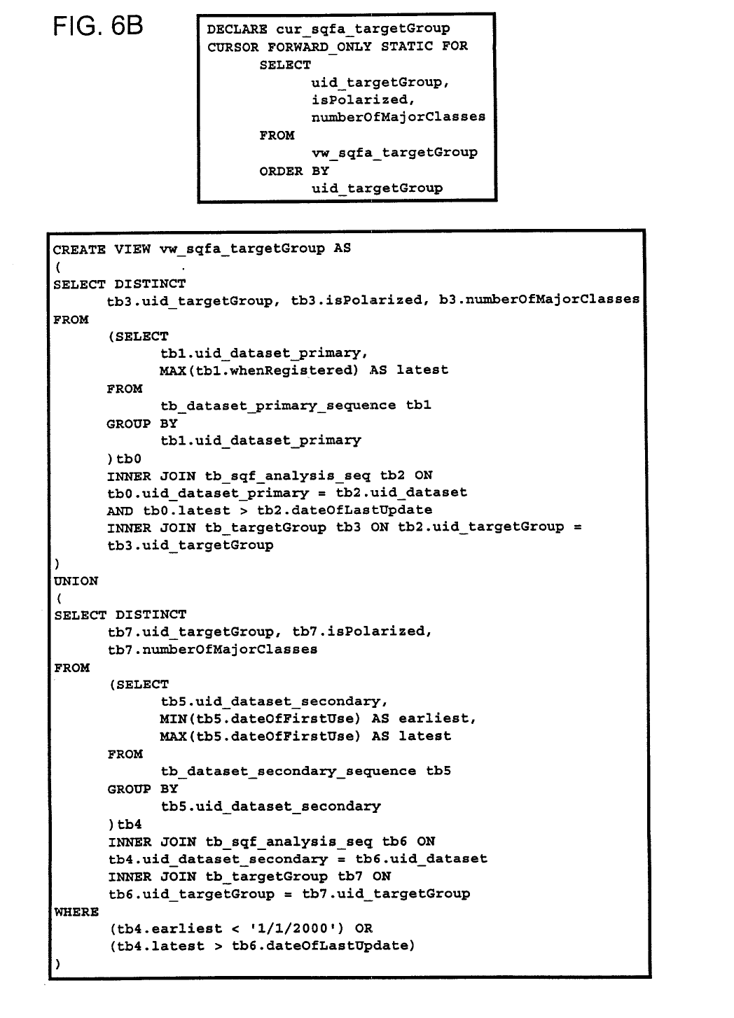

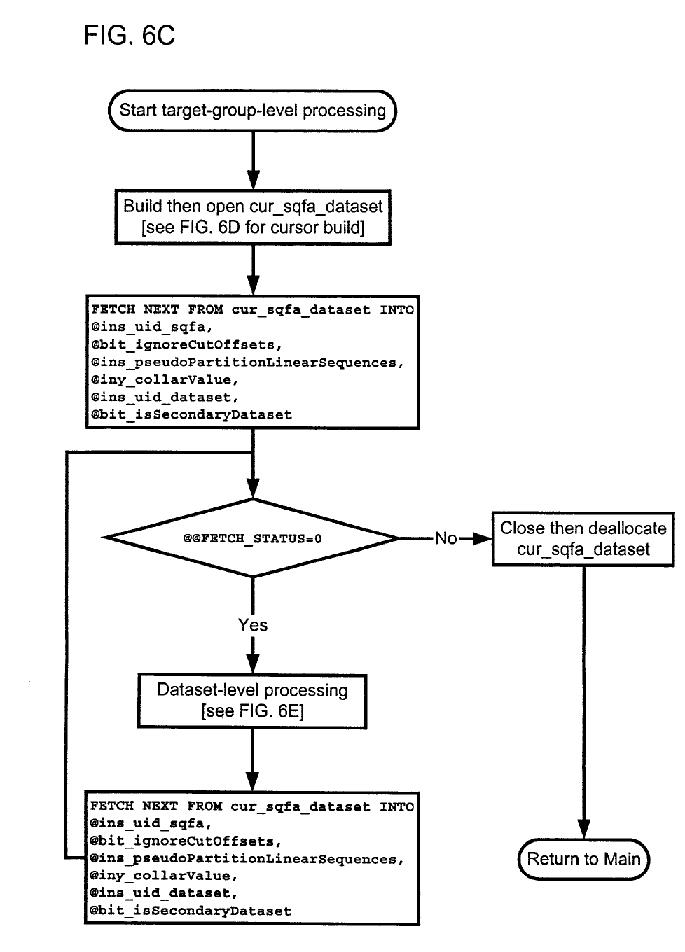

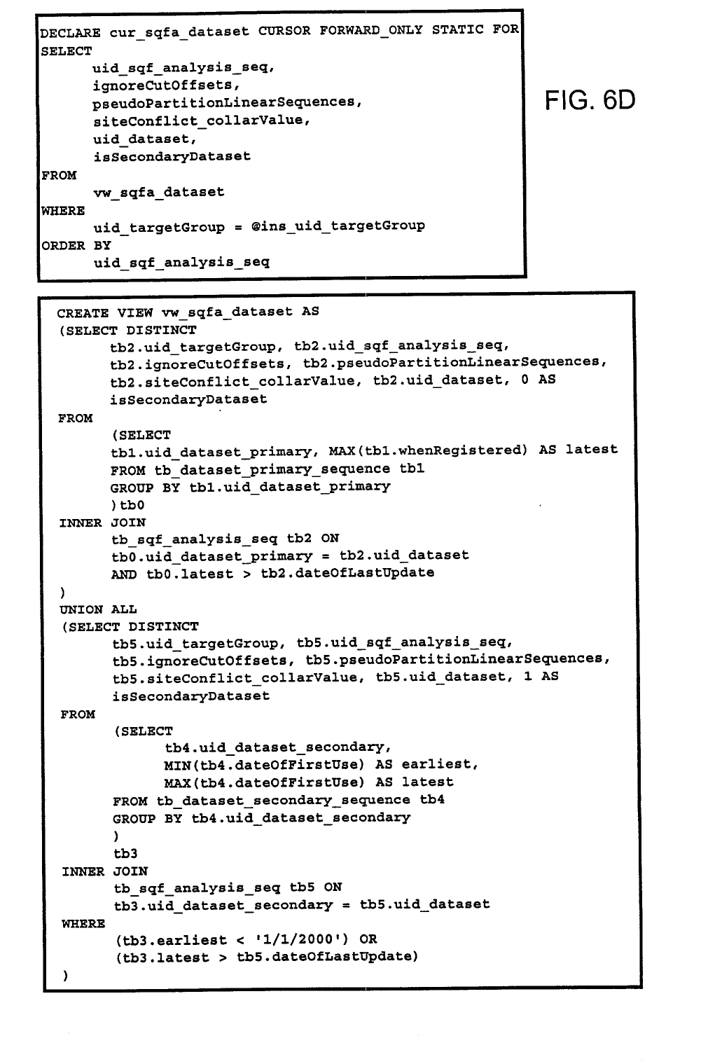

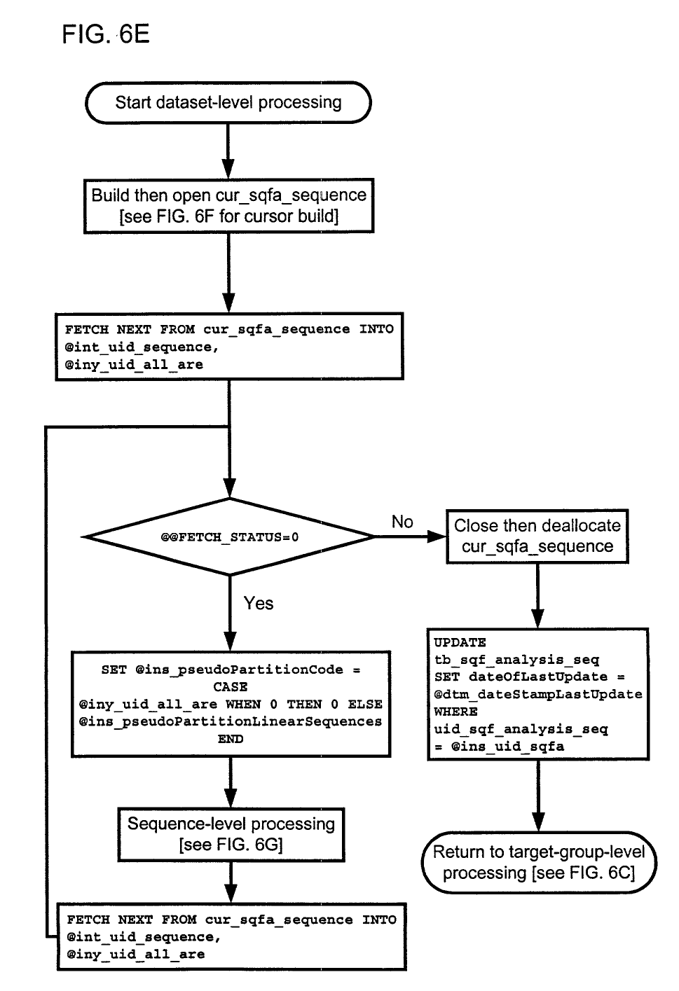

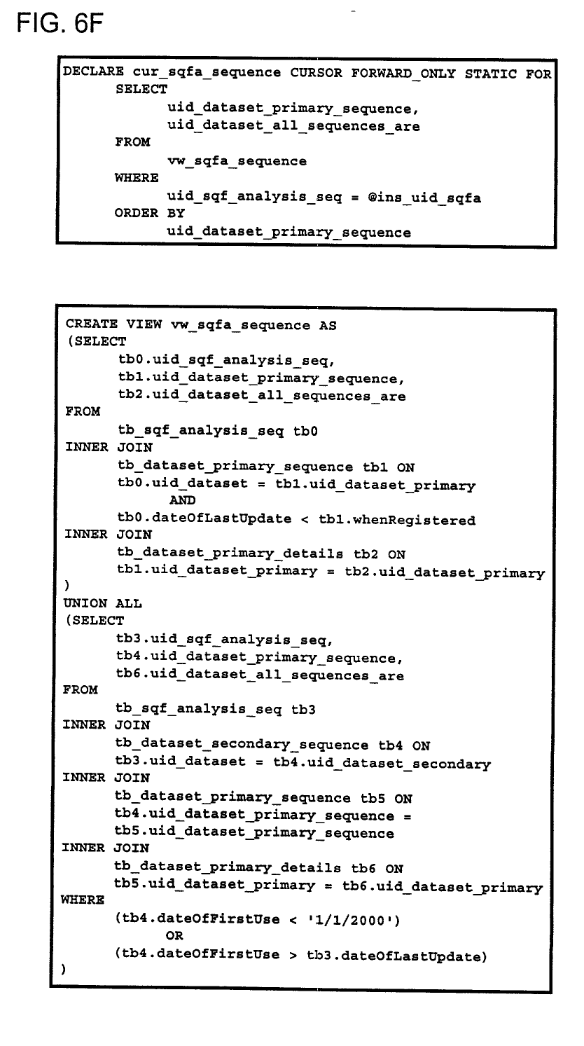

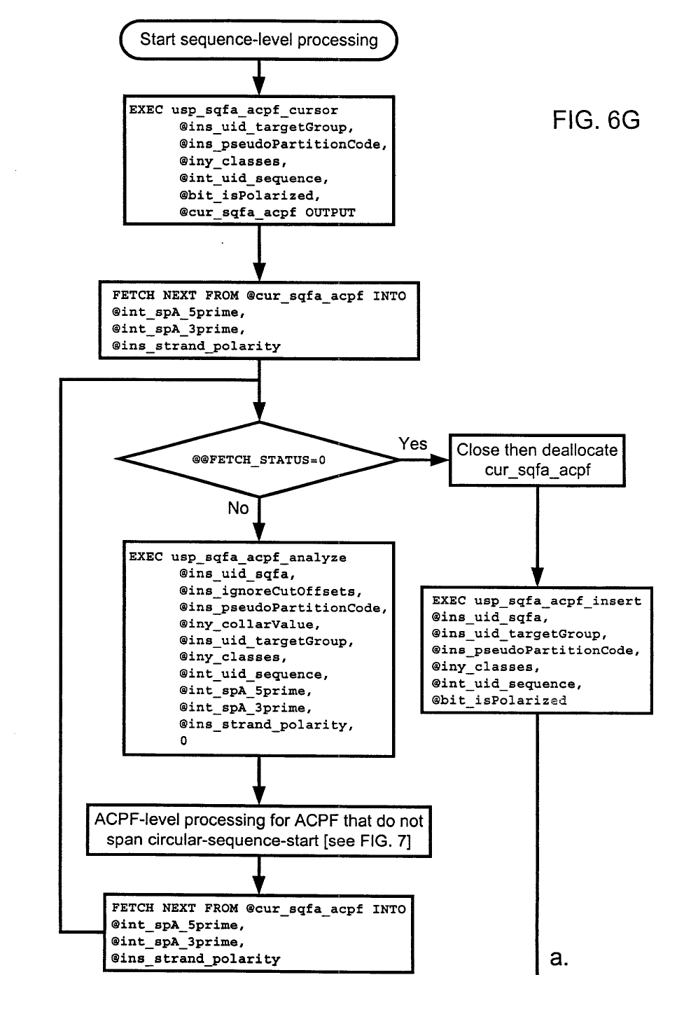

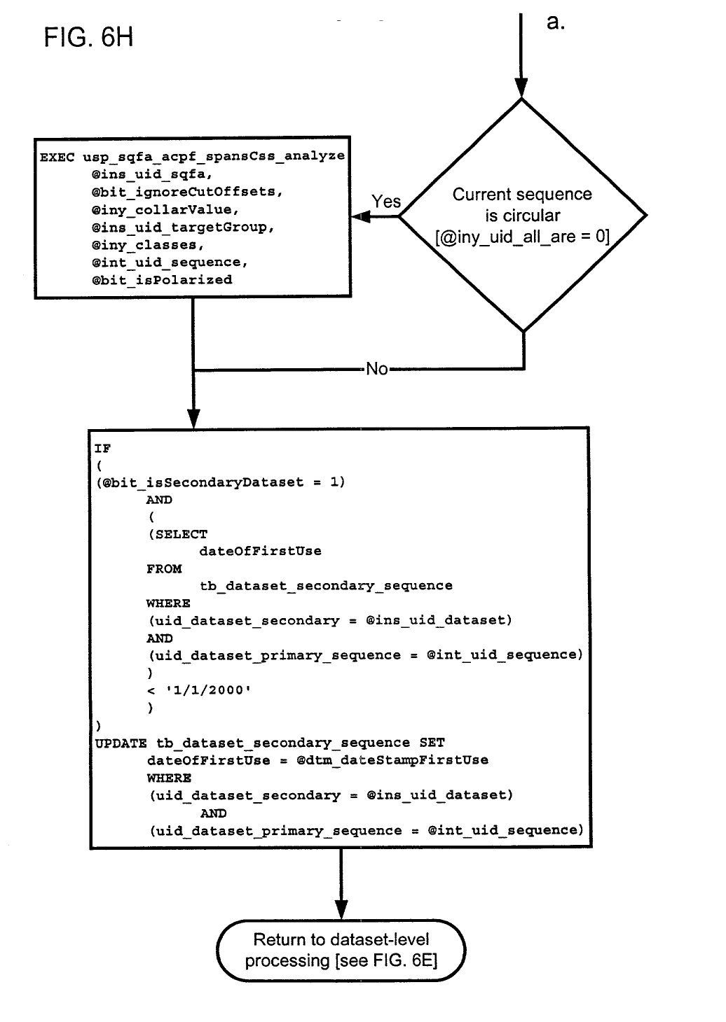

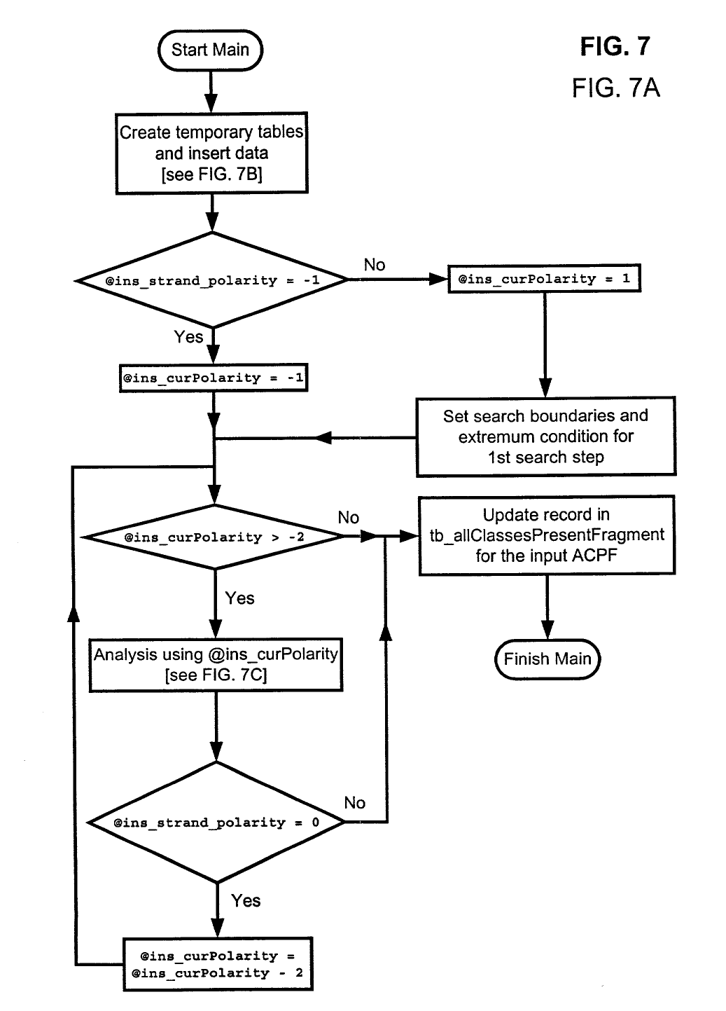

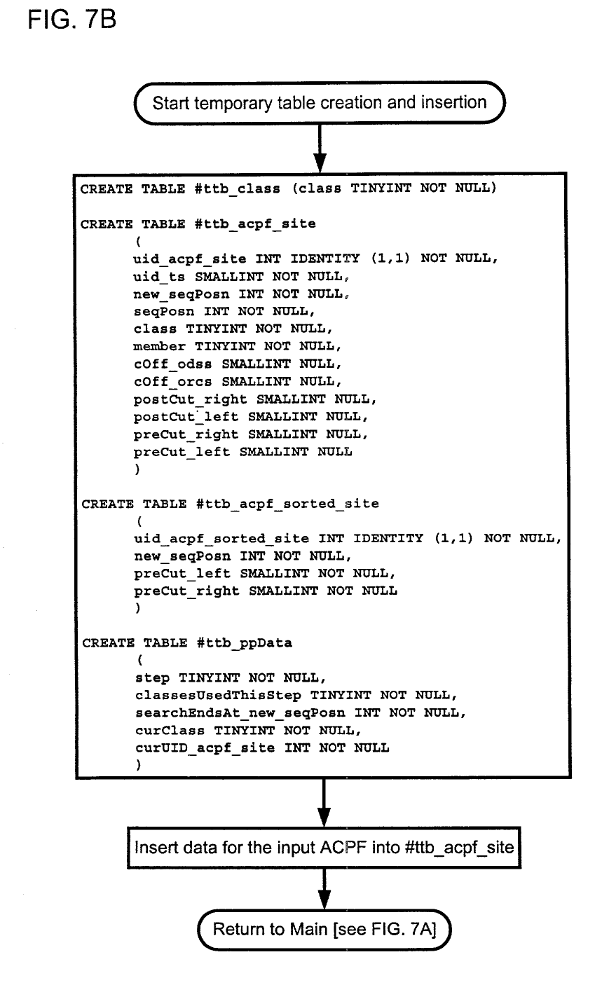

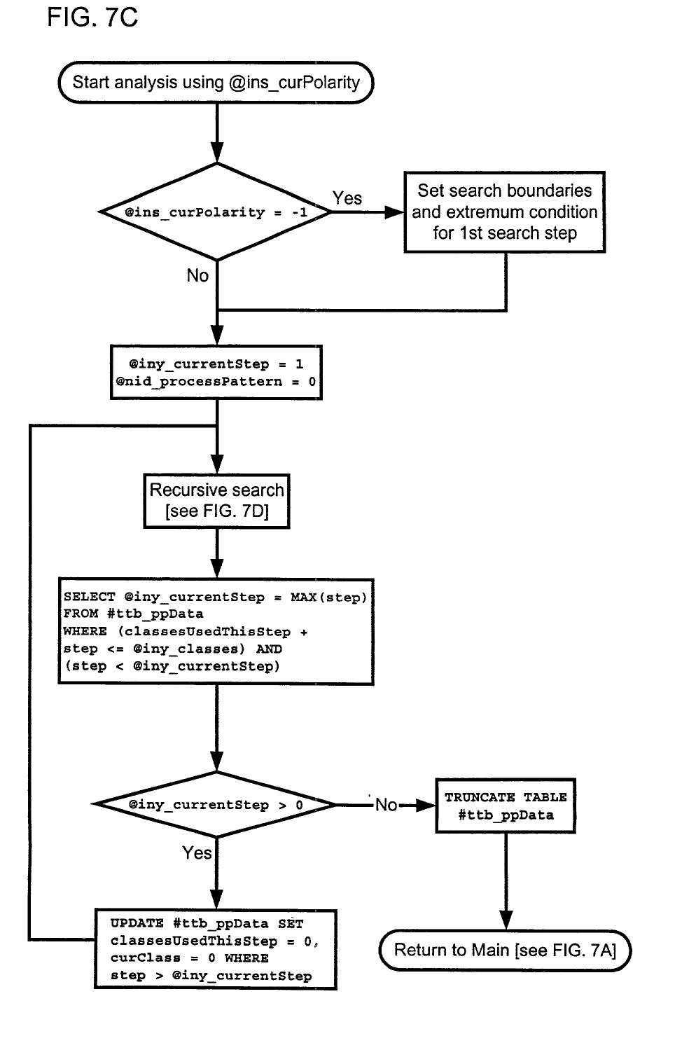

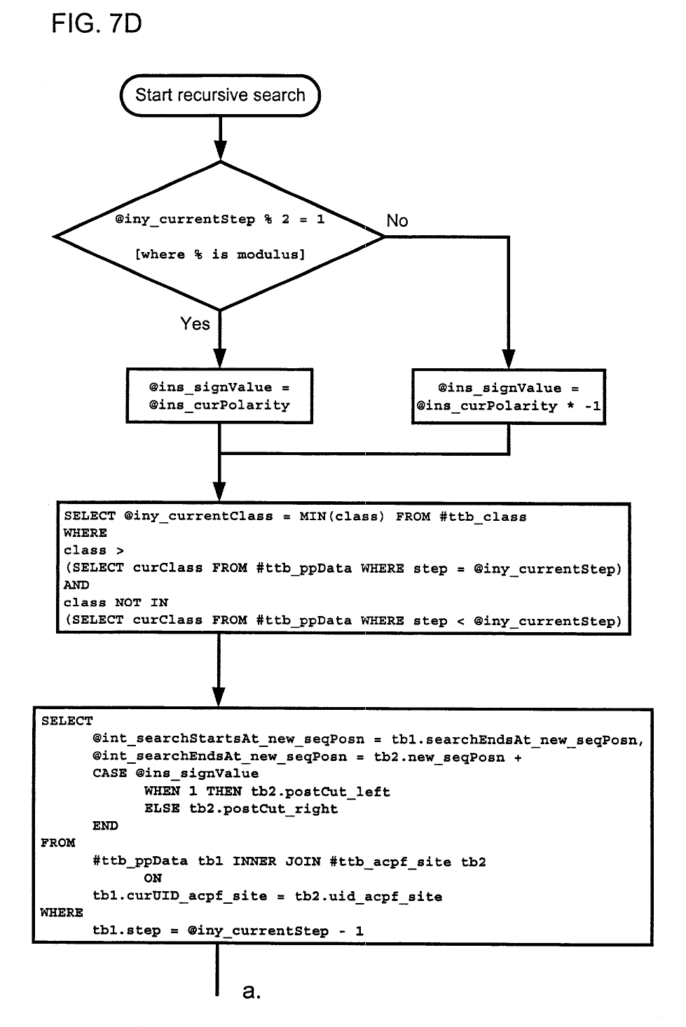

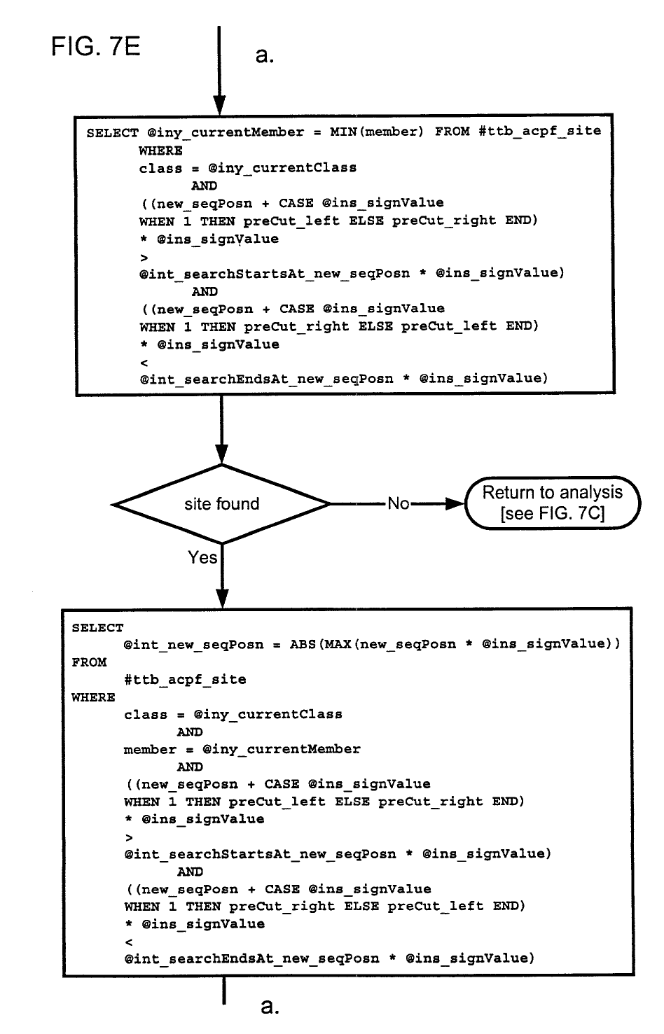

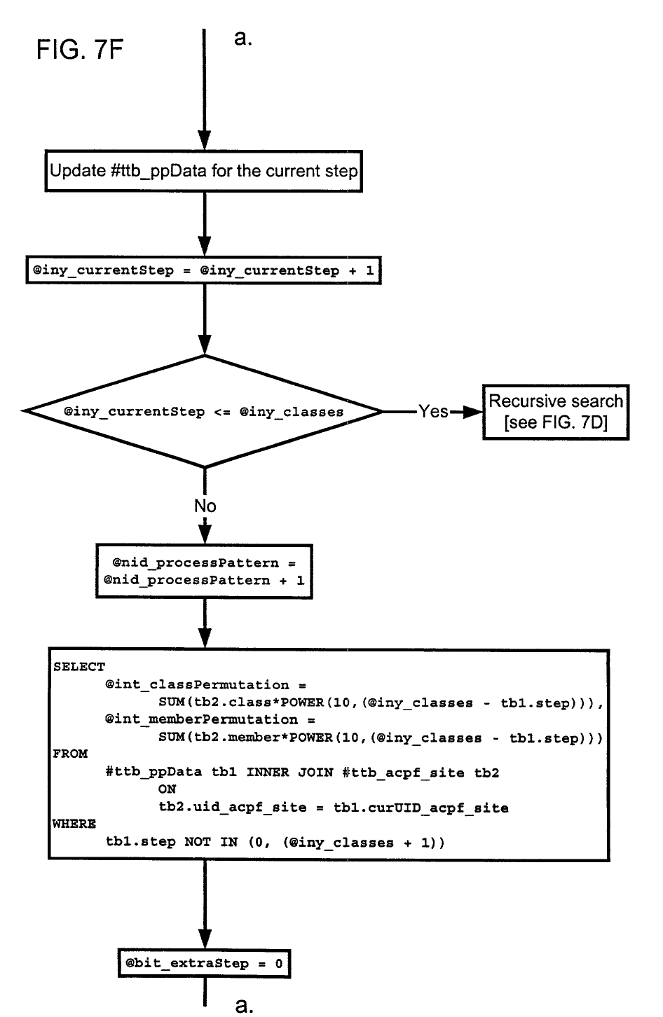

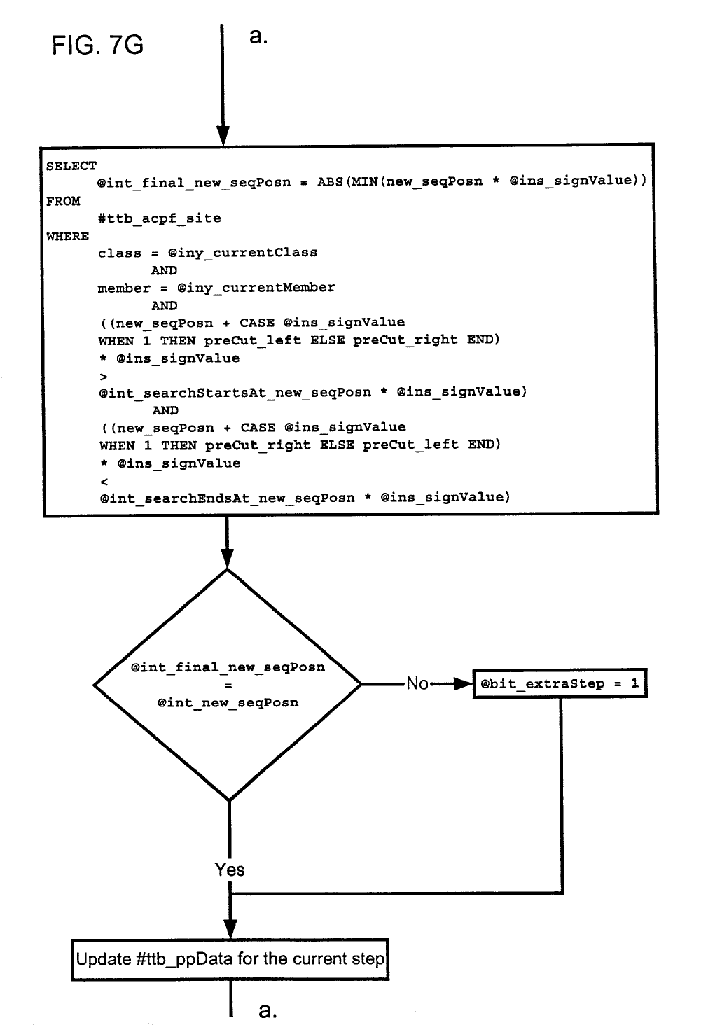

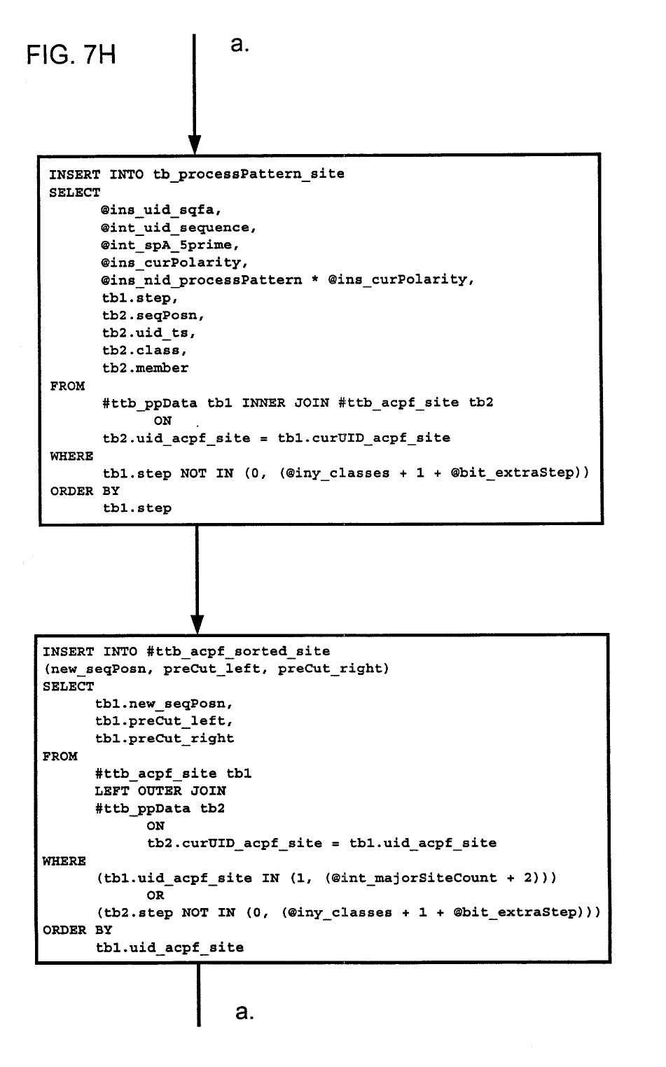

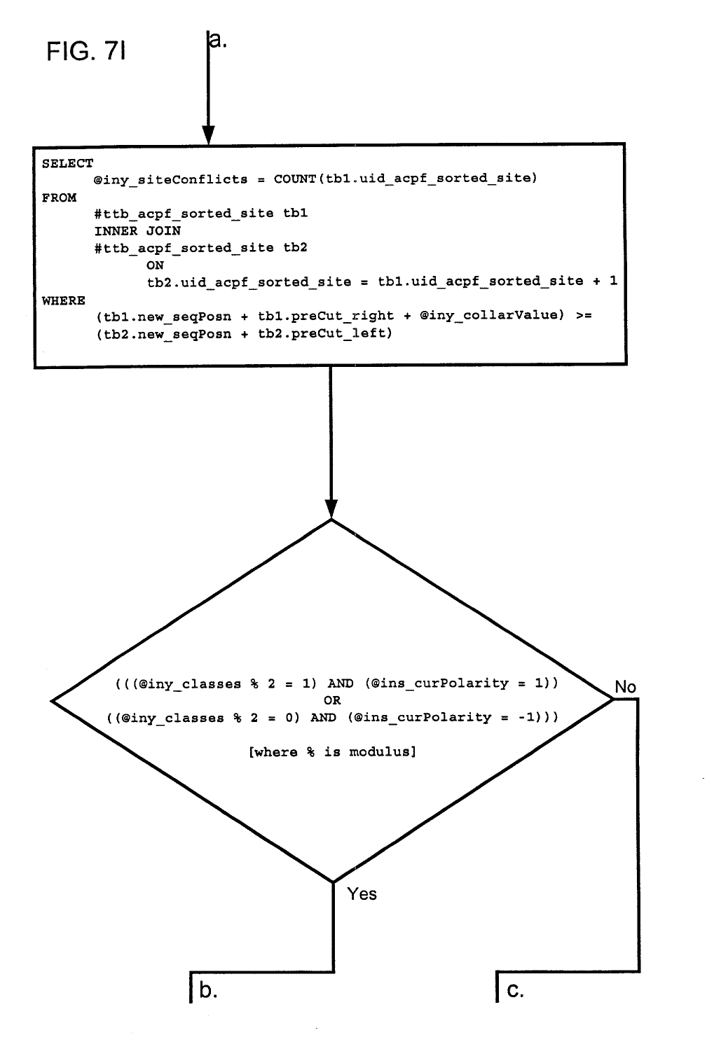

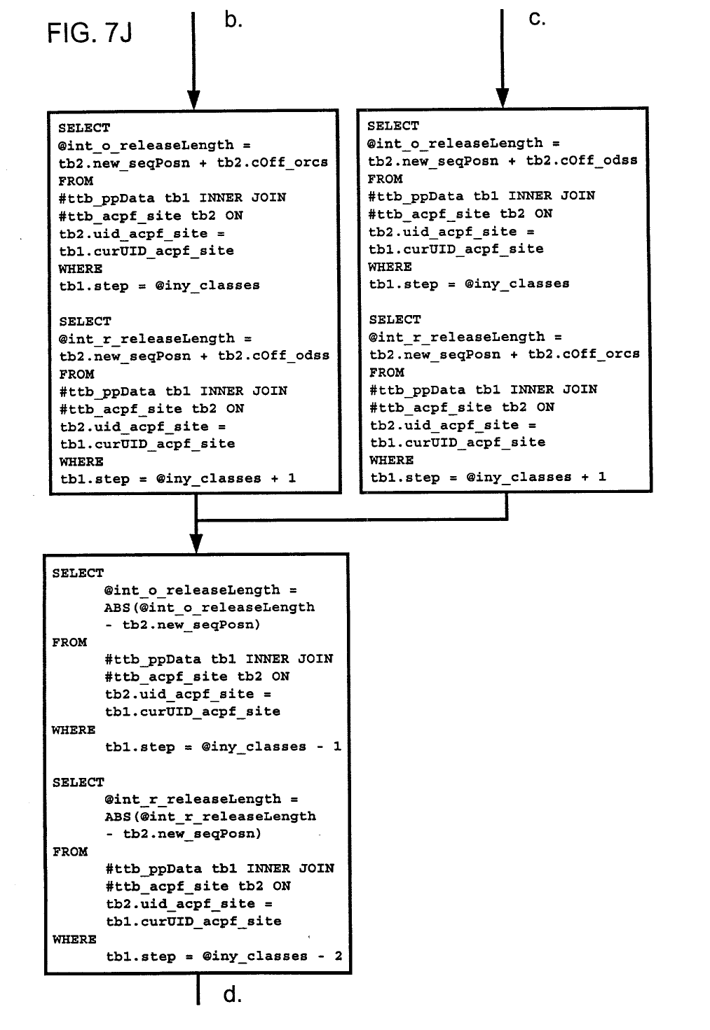

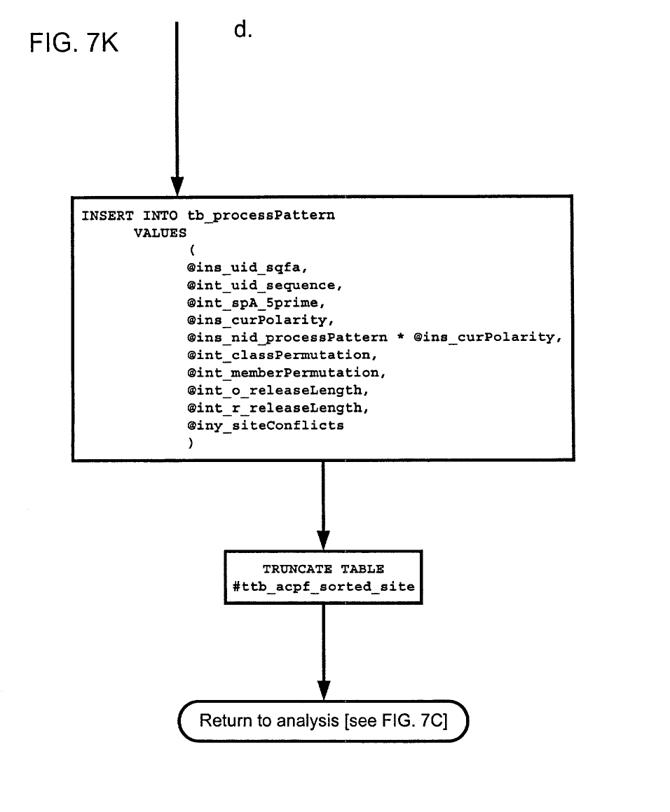

An SQF analysis uses a search target-group (Examples Tables 3-5) to detect “process-patterns”, typically in the sequences that comprise a set of biopolymer sequences (FIGS. 6 and 7). A search target-group typically contains a single “partition” search target, and a structured array of “major” search targets. Each column in the array of “major” search targets is known as a “major class”. These major classes can be referred to by letter designations such “B”, “C”, “D”, “E”, “F”, etc. or by the use of non-zero integers (1, 2, 3, . . . etc.) as index values. Each major class is comprised of a ranked set of a limited number of major search target members (e.g., B1, B2, B3, and B4 in class B). The specific number and identity of search targets and major classes chosen is flexible and depends on the particular application of the present invention.

The number of “major classes” in a search target-group determines the number of search steps required to define a process-pattern. Each search step: (i) is specific for a given major search class, where the major class for each step is selected from the major search classes that define the search target-group; (ii) proceeds in a specific direction over a process-defined, restricted region of the partition fragment; (iii) seeks the highest-ranked member of the current major search class in the current search region; (iv) if successful, truncates the current search region and limits the search region for the next search step; (v) is part of a process that defines a pattern, where for a given target-group, each site in the pattern indicates the presence of the site found-and the absence of higher-ranked members of the same major search class-in that site's process-defined search region.

In certain preferred embodiments, the procedure uses combinatorics. In these embodiments, the order of major search classes used to define process-patterns is permuted (e.g., [B, C, D, E, F] vs. [C, B, D, E, F]). Each partition fragment may be queried for the presence of all of the process-patterns that can be generated using all of the possible permutations of the major classes in the search target-group, and using both of the possible starting directions for the first search step (FIGS. 6 and 7). Thus, a well-designed search target-group comprised of a limited number of small search targets can query a genome at very high frequency.

A structured query fragment is simply a fragment bounded by two sites in a process-pattern. Typically, two SQFs adjacent to the search target site detected in the final search step are of most interest.

Individual search targets (Q) are relatively small strings of variable length (q) with a defined maximum possible length (zₘₐₓ), where the value of (zₘₐₓ) is the maximum length of the first-order substrings used during the import and storage of the input-strings in the relational database application as described earlier. Non-degenerate characters and non-degenerate variant forms of the degenerate characters used to define any search target (Q) are typically all be members of the character set used to define the input-strings from the primary dataset.

In certain embodiments, each search target (Q) typically defines at least one (Sᵢ) and at most two (Sᵢ and S₋ᵢ) search target-strings, and where in the latter case the two search target-strings (Sᵢ and S₋ᵢ) typically are structurally related forms of the search target (Q) that defines them. Search target strings are typically fully defined in an automated manner and stored in the relational database application of the present invention.

Typically, a search target group is a group of distinct, mutually non-coincident search targets, where none of a search target's possible search target-strings may be a substring of either of the possible search target-strings of another search target in the same search target group. Typically, each search target-group includes a single “partition” search target (Qa), often referred to as simply “target-A”, and an array-like set of “major” (i.e., non-Qa) search targets comprised of a limited number of major search classes, and where each major class (Cᵢ) of targets in (Gu) contains a limited number of ranked members (Qm_{i,j}) (where for a given major class Cᵢ, Qm_{i,1} is the highest-ranked member, Qm_{i,2} is the second highest-ranked member, and so on). In some embodiments, Qa may also effectively represent more than one search target, based on the definition of Qa's search target-strings; the latter, though typically the reverse complements of each other for non-palindromic search targets, do not need to be so defined, nor limited in number. The number (Jmaxᵢ) of members jᵢ) per major class (Cᵢ) may vary for each major search class defined by (Gu). The definition of the search target-group may, in the case of those search targets that can generate two search target-strings (Sᵢ and S₋ᵢ), specify that either (Sᵢ) or (S₋ᵢ) or both (Sᵢ and S₋ᵢ) are to be used in the search procedure described below. Search target-groups are defined and stored in the relational database application of the present invention.

Typically, a search target group includes between 3 and 9 major search classes, and more typically between 3 and 6 major search classes. In one preferred embodiment, the search target-group contains 5 major search classes. Typically, a major search class contains between 1 and 9, and more typically between 3 and 6, search target members. The SQF analysis simulation algorithm described below can be used to determine the number of major search classes and the specific search targets required for a given SQF analysis to yield a desired number of SQFs with a desired size distribution, based on the size of the primary dataset of interest and the mean fragment lengths associated with the search targets in the search target-group.

In a preferred embodiment, one or more search targets (Qᵣ) in a search target-group (G) is or contains a distinct recognition sequence for a cleavage effector. A “cleavage effector” in this specification refers to an enzyme or enzymatic process, or a chemical reagent or chemical process, or a physical process, that can create a double-stranded cleavage point in a polynucleotide in a sequence-specific manner at, or a known distance from, the recognition sequence of the said enzyme, reagent, or process. In certain preferred embodiments, the cleavage effector is a type II restriction endonuclease.

Typically for this embodiment, the relational database application contains information for each search target Qᵣ about the cut-offsets (CO) associated with the cleavage effector whose recognition sequence is, or is found within, Qᵣ (Examples Table 3). Preferably, a dataset-strand (CO_{ds}) cut-offset and a reverse-complement strand (CO_{rc}) cut-offset are specified for each Qᵣ. A variable may be defined, for example, CO_{ds} and CO_{rc} to set the distances from the start of a scored site for Qᵣ to the position where the 3′-hydroxyl-bearing nucleotide would be found on the dataset strand or the reverse-complementary strand, respectively, after a sequence-specific, double-stranded cleavage event. If Qᵣ is non-palindromic, the relational database application typically contains CO_{ds} and CO_{rc} values for both of the two possible search target-strings (Sⱼ) and (S₋ⱼ) defined by Qᵣ. For each Qᵣ, it is possible to calculate the effective functional boundary required by, and resulting from, a sequence-specific, double-stranded cleavage event at any Qᵣ site, using the values of CO_{ds}, CO_{rc}, and the length of Qᵣ for any search target (Qᵣ ). In a preferred embodiment, there may be one or more search targets (Qᵣ ) in a search target-group (G) where each Qᵣ comprises a distinct recognition sequence for a sequence-specific endonuclease, including a Type II restriction endonuclease.

A specific non-limiting example of a search target-group is shown in Examples Table 5 (see also Tables 3 and 4). In this search target-group (target-group ID #1), the partition search target is the string representing sites in DNA recognized by the restriction endonuclease Ssp I; the ordered members of one major class (class B) are the strings representing sites in DNA recognized by the restriction endonucleases Acc65 I, Pae I, Aft II, and Stu I; the ordered members of another major class (class C) are the strings representing sites in DNA recognized by the restriction endonucleases BstE II, Mfe I, Avr II, and Hind III; the ordered members of another major class (class D) are the strings representing sites in DNA recognized by the restriction endonucleases Bsh1365 I, Sca I, Bpu1102 I, and BsrG I; the ordered members of another major class (class E) are the strings representing sites in DNA recognized by the restriction endonucleases Spe I, Cfr9 I, Bcl I, and Nco I; and the ordered members of another major class (class F) are the strings representing sites in DNA recognized by the restriction endonucleases BamH I, Eco32 I, Bgl II, and Xba I.

In certain embodiments, there may be one or more search targets (Qᵣ ) in a search target-group (G) where each Qᵣ comprises a recognition sequence for an enzyme or enzymatic process, or a chemical reagent or chemical process, or a physical process, that can modify one or more of the mononucleotides in a polynucleotide in a sequence-specific manner at, or a known distance from, the recognition sequence associated with the enzyme, reagent, or process. In a specific embodiment, the modification is methylation of one or more of the mononucleotides; whereas in another embodiment the methylation is specific for cytosine residues in a polynucleotide. In certain embodiments, Qᵣ comprises a recognition sequence for a sequence-specific cytosine methylase enzyme.

In certain embodiments where the set of strings are polynucleotides, there may be one or more search targets (Qᵣ ) in a search target-group (G) where each Qᵣ comprises a sequence that is known, or suspected of having, some structural, functional, or regulatory significance in some naturally-occurring or experimental biological context, where such a context is defined as the presence of the sequence in a specified replicating (e.g., virus, episome or chromosome) or non-replicating polynucleotide entity, in a specified species, in a specified cell type, at a specified developmental stage, under a specified set of conditions, and the like.

Typically, the fraction of degenerate characters that are present in a search target is not allowed to exceed a certain value. Typically, a search target with a positive search target-string of length (z) (where z≦zₘₐₓ in embodiments that utilize primary dataset substrings of maximum length zₘₐₓ), the ratio (“relative degeneracy units” [RDU]/z) may not exceed 0.5, where the following commonly used nucleotide symbols are assigned the indicated values for (RDU): N=1;[any of R, Y, W, S, K, M ]=0.5; [any of B, D, H, V]=0.75; and [any of A, C, G, T]=0. In one preferred embodiment, where the values of (zₘₐₓ) and (yₘₐₓ) are 18 and 6, respectively, the RDU also may not exceed 6 in a search target.

Where the set of strings is a set of characters representing polynucleotide sequence data, the array-like set of major search targets in the search target-group (Gu) to be used in an SQF analysis (U) using dataset (Du) may be selected so that the array-like set is “symmetrically descending”, based on the mean recurrence (or fragment) length (m_{i,j}) in (Du) associated with each major search target (Qm_{i,j}) in the array (see Examples Table 5; see also Table 6, Dataset #1). “Symmetrically descending” means that for each member index value (jᵢ) common to two or more major search classes (Cᵢ) in (Gu), the values of (m_{i,j}) are as closely matched as possible, e.g., if the number of major search classes (Mᵤ) in (Gu) is five, then for (jᵢ)=1, (m_{1,1})≈(m_{2,1})≈(m_{3,1})≈(m_{4,1})≈(m_{5,1}) and similarly for all remaining values of (jᵢ); and where the definition of “symmetrically descending” also states that for each of the major search classes (Cᵢ) in (Gu), e.g., for (i)=1, and assuming that there are four members in this major search class, then (m_{1,1})>(m_{1,2})>(m_{1,3})>(m_{1,4}), and similarly for all of the remaining values of (i).

In one preferred embodiment, search targets (Q) used in the relational database application represent oligonucleotide sequences that may be of interest in the definition of, and search for, process-patterns and SQFs.

In another embodiment, search targets (Q) used in the relational database application computationally generate the full definition of their respective search target-string derivatives. Typically, this information is stored in a table in the relational database application. In this embodiment, each search target (Qₚₐ) that is a palindromic sequence with a positive target-string (Sᵢ) may define only one target-string derivative (Sᵢ); whereas each search target (Qₙₚₐ) that is a non-palindromic sequence with a positive target-string (Sⱼ) may define one target-string derivative (S₋ⱼ) and computationally generates a second target-string derivative (S₋ⱼ), where (S₋ⱼ ) is the reverse-complement of the positive target-string (Sⱼ), and where the value (−J) of the primary key assigned to (S₋ⱼ) is the negative value of the primary key value (J) assigned to the positive target-string (Sⱼ).

In certain embodiments, a simple modification of the software used in other preferred embodiments allows SQF analyses where the naturally occurring ends of a linear string representing a linear entity (e.g., the DNA sequence of a chromosome) may be used to mimic the functionality of a partition search target, and thus the entire length of the said linear sequence is treated as one single partition fragment during SQF analyses (see FIGS. 6 and 7; see also the field “pseudoPartitionLinearSequences” in “tb_sqf_analysis_seq” in FIG. 2B).

C) Processing of qualifying substrings.

1.) General considerations.

The bioinformatic embodiments of the present invention typically include the definition of searching procedures, or structured query fragment analyses, described herein, that define and locate process-patterns and SQFs. Search results may be stored in the relational database application (FIG. 2). Typically each SQF analysis specifies a unique combination of a single dataset, either a primary dataset (Dp) or a secondary dataset (Ds), and a single search target-group, together with other information that may be relevant to the SQF analysis (Examples Table 11).

The searching procedures, also called SQF analysis methods, typically involve processing each of the qualifying partition fragments obtained from the set of strings in the dataset (FIG. 7). This processing involves both a series of search target site discovery steps, and the progressive, step-wise delimitation of the available search region, based on the location of the search target sites discovered during the process.

In certain preferred embodiments, processing of a given string in the set of strings begins by determining which partition fragments in the said string are “all-classes-present fragments” or ACP fragments, where each ACP fragment contains one or more sites for at least one member of each major search class in the search target-group used in a given analysis (Examples Tables 13 and 15). All-classes-present fragments are also referred to as qualifying substrings and qualifying partition fragments in this specification.

Typically, each ACP fragment is examined using all of the possible permutations of the major search classes of the search target-group to determine the presence of process-patterns therein (FIG. 7). Thus, the “pattern” of a process-pattern is a set of search target sites in a partition fragment, with only one search target site from each major class present in the pattern (Examples Tables 14 and 15). Typically, each search step in the determination of a process-pattern is performed with a defined polarity (direction) and extremum condition, where for each search step the site chosen to contribute to the pattern is the highest-ranked member of the current search class (as defined by the major class permutation used for the search) that is within the current search area and satisfies the extremum condition adopted for the search. The extremum condition typically states that if there are two or more sites for the highest-ranked member of the current search class in the current search area, the site furthest from the starting point of the search is chosen. Typically, this processing results in the identification of process-patterns and their structured query fragment derivatives in the ACP fragments that are obtained from the set of strings (Examples Tables 14 and 15).

The following paragraphs provide a more detailed description of the searching procedure of certain preferred embodiments of the present invention, including the definition and typical values of certain variables.

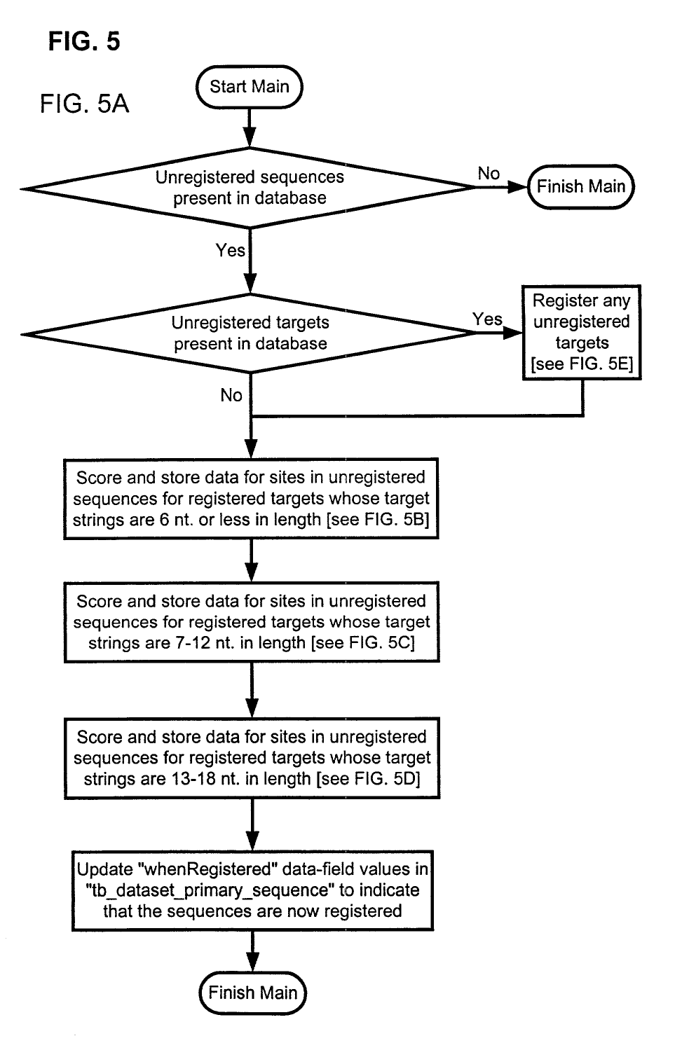

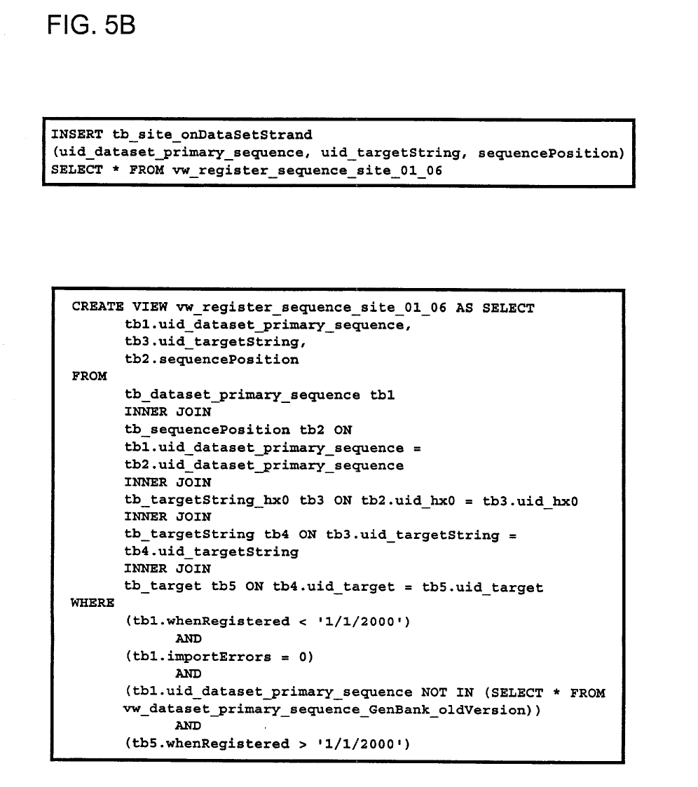

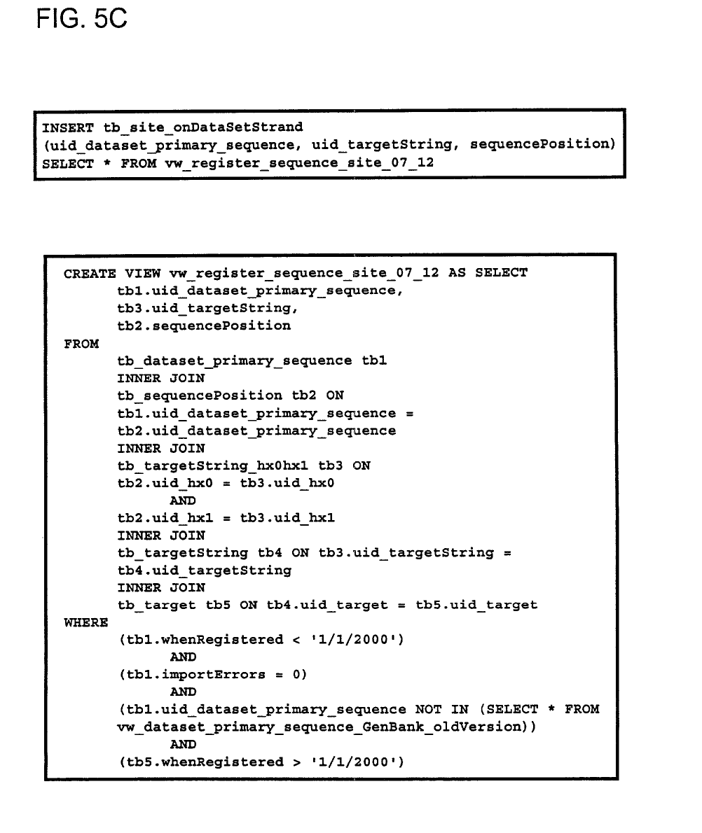

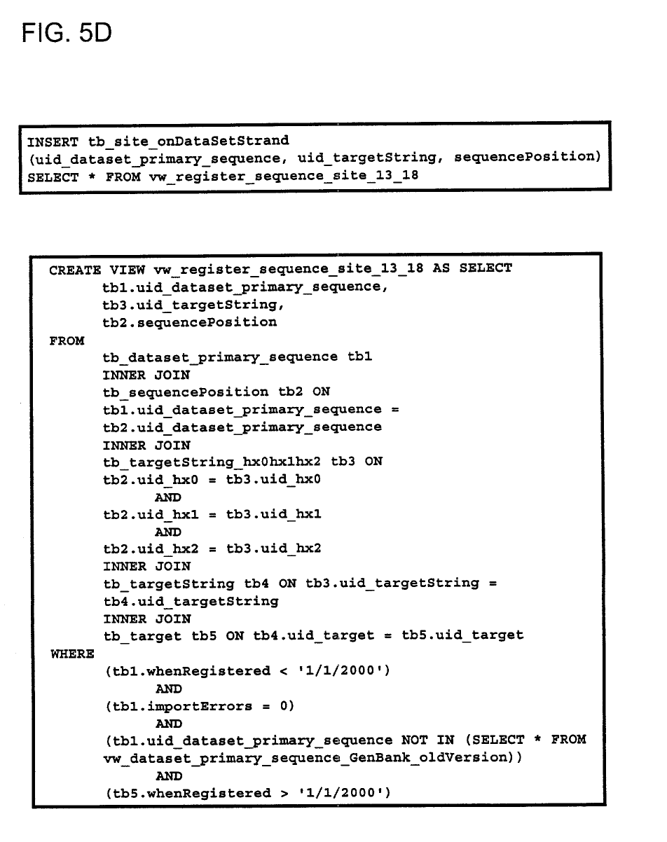

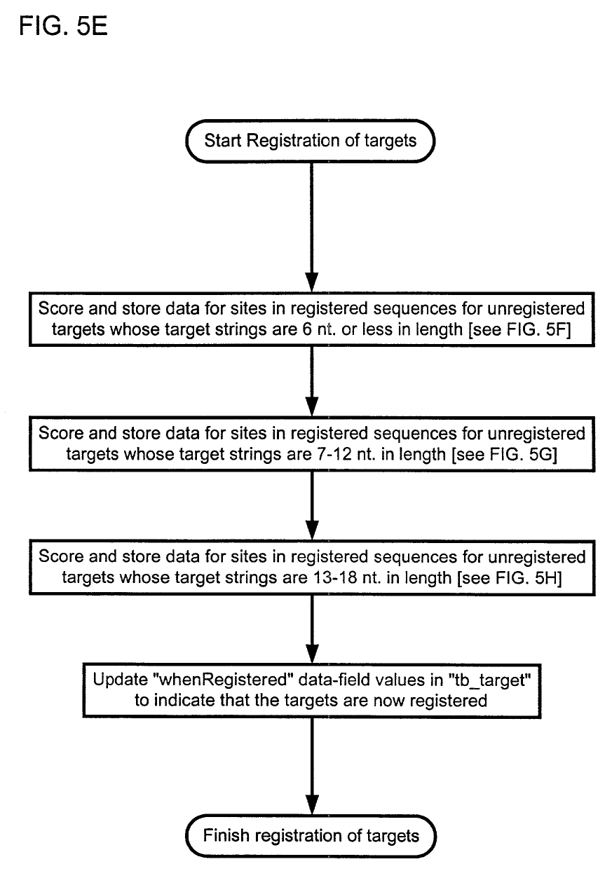

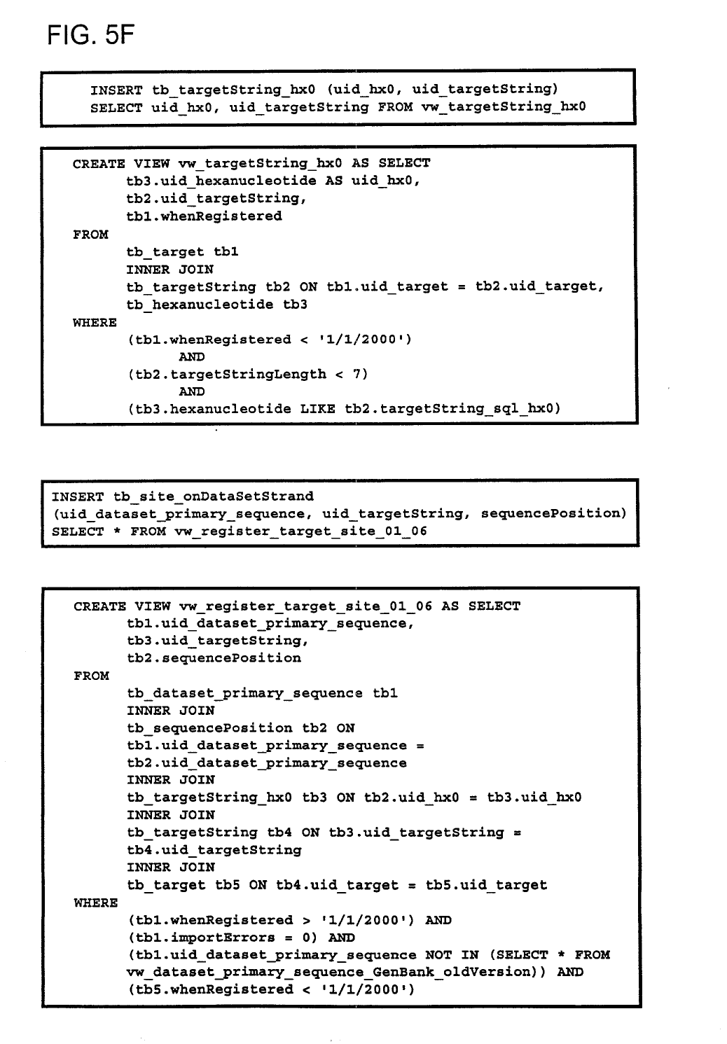

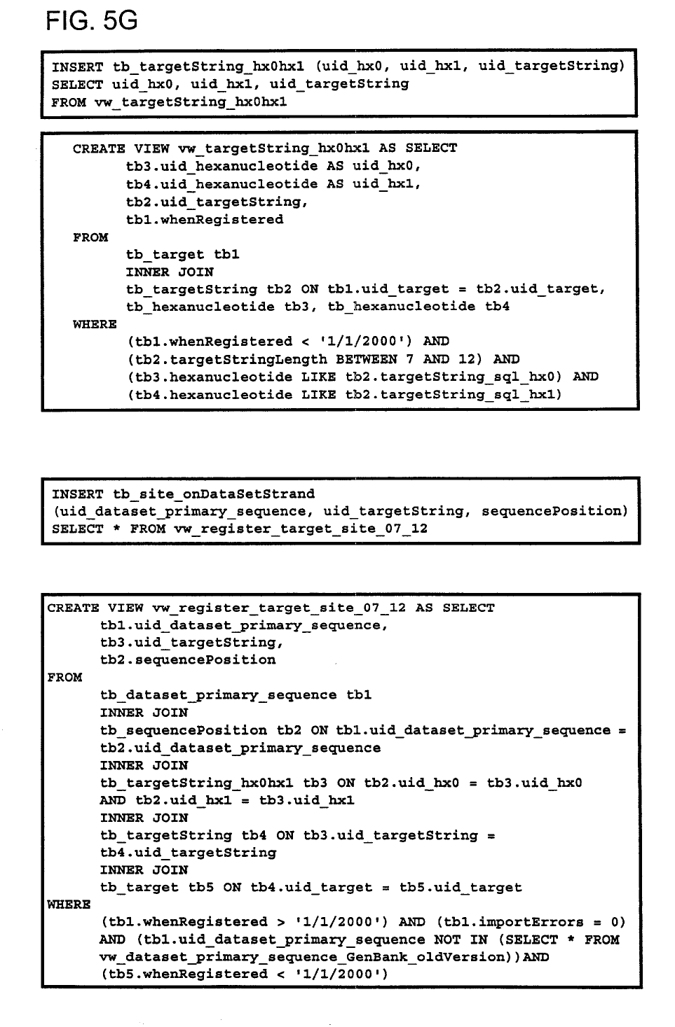

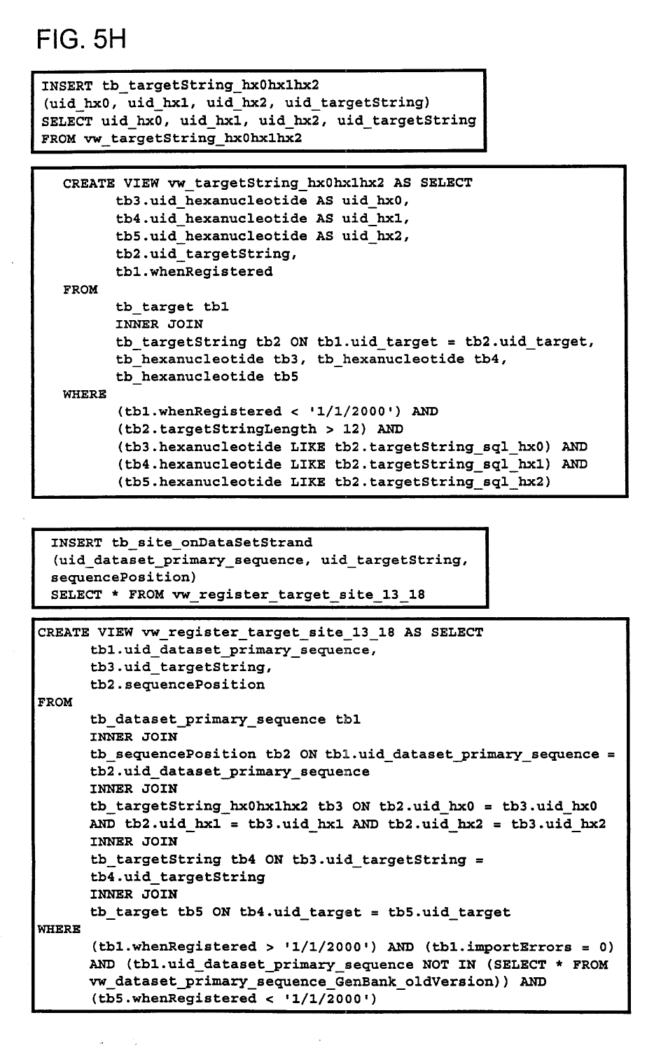

For an SQF analysis (U) defined by a dataset (Du) and a search target-group (Gu) with Mᵤ major classes, the searching process may initially comprise the “scoring” of the “partition” search target (Qa) in all of the strings in (Du), where the process of “scoring” any search target (Q) is defined as the determination of the positions of all of the instances of the occurrence of the search target-strings {either (Sᵢ) or (S₋ᵢ), or both (Sᵢ and S₋ᵢ)} that may be defined by Q and are present in the strings in Du. The requirement to search for either Sᵢ or S₋ᵢ, or both Sᵢ and S₋ᵢ, may be part of the definition of each search target's (including Qa's) membership in a search target-group such as (Gu). The rapid scoring of relevant search target sites is facilitated by a design feature of the relational database application developed for the present invention. This design feature is the “registration” of newly acquired input-strings and newly designed search targets, whereby a table (“tb_site_onDatasetStrand”, FIG. 2D) is maintained that contains the scored positions of all of the instances of the occurrence of the search target-strings of all of the registered search targets in all of the registered input-strings (FIG. 5).

Next, the search may comprise scoring of the major search targets (Qm_{i,j}) in all of the strings in Du, where for each Qm_{i,j} the requirement to search for either Sⱼ or S₋ⱼ , or both Sⱼ and S₋ⱼ is defined in Gu.

Next, a determination may be made of those “partition” (Qa-Qa) fragments, described above, in Du that qualify as “all-classes-present fragments”, also called ACP fragments or simply ACPF (Examples Tables 13 and 15). The set of Qa-Qa fragments in Du is defined as all of the substrings therein that are bounded at each end by either Sᵢ or S₋ᵢ, as defined for Qa in Gu, and where either Sᵢ or S₋ᵢ, or both Sᵢ and S₋ᵢ, may not be present between the Qa sites at either end of a Qa-Qa fragment. The definition of a Qa-Qa fragment includes any Qa-Qa fragment that may be derived from a circular string of characters where the Qa-Qa fragment spans the start site of a linear string of characters that represents the circular string of characters. All Classes Present (ACP) fragments are a subset of the set of Qa-Qa fragments that can be derived from Du. Every Qa-Qa fragment that can be derived from Du and that contains one or more instances of the occurrence of at least one member (Qm_{i,j}) of each major search class (Cᵢ) in Gu is an ACP fragment.

Next, the search process of the present invention typically involves a hierarchical, pre-emptive search for all of the process-pattern entities that are present in each of the ACP fragments derived from the dataset Du using the search target-group Gu with Mᵤ major classes (FIG. 7). The “pattern” of a process-pattern in this embodiment is an ordered set of Mᵤ search targets (Qm_{i,j}) in an ACP fragment, with only one search target (Qm_{i,j}) from each major class (Cᵢ) present in the set, and where the order of the search targets recorded for a process-pattern defines their order of discovery, and is thus a self-documenting record of each step in the definition of the search process that yielded the pattern (Examples Table 14). Furthermore, for each site in the process-pattern, higher-ranked members of the same major search class must be absent within the relevant search area of the ACP fragment (Examples Table 15). The relevant search area is defined by the search steps of the process-pattern, which as mentioned earlier is self-documented by the process-pattern's recorded description.

Thus, a process-pattern's full definition includes both the “pattern”described above and a step-wise delimitation “process”, where each of the (Mᵤ) search steps in this process has a defined polarity and extremum condition. A left-to-right polarity (+1) or a right-to-left polarity (−1) may be used in the definition of a search step. Additionally, a search step is defined by a furthest-right extremum condition (furthest-right qualifying site in the relevant search area of the ACP fragment) or a furthest-left extremum condition (furthest-left qualifying site in the relevant search area of the ACP fragment) so as to deal with the possibility of multiple instances of the highest-ranked member of the current major search class being present in the relevant search area.

For a preferred search procedure of the present invention, the first major-class-specific search step occurs at the very start of the process-pattern search, and subsequent search steps occur after the highest-ranked member of the current major search class that satisfies the current extremum condition is found. Each major-class-specific search step restricts the region of the ACP fragment where the next major-class-specific, pre-emptive target-search takes place. Typically, all possible class-permutations of the M^{u} major classes (C₁, C₂, . . . , C_{M}) are used for process-pattern definitions, and the corresponding search for all of the possible process-pattern entities in each of the ACP fragments derived from the dataset (Du). As described above, a structured query fragment (SQF), obtained by the SQF analysis (U) using the dataset (Du) and the search target-group (Gu) as described in more general terms above, is defined as a substring within one of the resulting ACP fragments, where the SQF's termini are any two search target sites that are part of the set of Mᵤ sites in a process-pattern entity that can be derived from the ACP fragment (Examples Table 15). The definition of the search target-group (Gu) together with the said process-pattern entity's definition are integral parts of the definition of the SQFs that can be derived there from.

2) Search Procedures with Polarized Search Target-Groups

The search procedure of the present invention may be performed with polarized search target-groups. The use of polarized search target-groups may be particularly valuable in bioinformatic studies of chromosomal translocations, genome rearrangement, inversion mutations, etc., where the strand-polarity of DNA regions between sites in a process-pattern entity may be uncertain.

For certain embodiments, only the dataset strands of sequences are scored for the presence of search targets. The scoring of the presence of a non-palindromic search target (Qₙₚₐ) with positive target-string (Sⱼ) may be unlimited (both Sⱼ and S₋ⱼ are scored), or limited to scoring either Sⱼ or its reverse-complement S₋ⱼ. Thus, although only the dataset strands of sequences are scored for the presence of search targets, the polarized scoring of either Sⱼ or its reverse-complement S₋ⱼ on the dataset strand of a sequence effectively allows for the polarized scoring of either S₋ⱼ or its reverse-complement Sⱼ, respectively, on the reverse-complementary strand of the said sequence.

For the search procedures of the present invention, non-polarized search target-groups or polarized search target-groups may be used in SQF analyses. A polarized search target-group (Gₚ) differs from a non-polarized search target-group (Gₙₚ) in that Gₚ contains one or more non-palindromic search targets (Qₙₚₐ), and further, one or more of the said non-palindromic search targets in Gₚ must be assigned a search target polarity of +1 or −1 in the definition of Gₚ. Other non-palindromic search targets in Gₚ may be assigned search target polarities of zero in the definition of Gₚ. However, a palindromic search target (Qₚₐ) may only be assigned a search target polarity of zero in the definition of either a non-polarized or a polarized search target-group.

An SQF analysis (Uₙₚ) performed using a non-polarized search target-group (Gₙₚ) allows two possible strand-polarities for the initial search step of an ACP fragment; a left-to-right polarity (+1), or 5′-3′ relative to the dataset strand, or a right-to-left polarity (−1), 3′-5′ relative to the dataset strand (or 5′-3′ relative to the reverse-complement strand). However, the definition of an ACP fragment in Uₙₚ is strand-independent, i.e., all search targets in Gₙₚ have search target polarities of zero, and thus ACP fragments obtained using Gₙₚ have strand-polarity zero. Furthermore, the definition of an ACP fragment in Uₙₚ does not limit the possible polarities for the initial search step, and thus process-pattern entities in Uₙₚ may have strand polarities of either +1 or −1.

The assignment of a search target polarity of zero to a non-palindromic search target (Qₙₚₐ) in either a polarized search target-group or a non-polarized search target-group specifies that both of the search target-strings Sⱼ and S₋ⱼ of Qₙₚₐ are used to score the occurrence of (Qₙₚₐ).

An SQF analysis (Uₚ) performed using a polarized search target-group (Gₚ) allows two possible strand-polarities for the initial search step of an ACP fragment; a left-to-right polarity (+1), or 5′-3′ relative to the dataset strand; or a right-to-left polarity (−1), 3′-5′ relative to the dataset strand (or 5′-3′ relative to the reverse-complement strand). However, the definition of an ACP fragment in Uₚ is strand-dependent, i.e., some or all of the search targets in Gₚ do not have search target polarities of zero, and thus ACP fragments obtained using Gₚ may only have non-zero strand-polarities of either +1 or −1.

The assignment of a search target polarity of +1 to any non-palindromic search target (Qₙₚₐ) in the definition of Gₚ specifies that in the search for ACP fragments of strand-polarity +1, only the positive target-string (Sⱼ) of the search target (Qₙₚₐ) is used to score an occurrence of Qₙₚₐ, and occurrences of the negative target-string (S₋ⱼ) are ignored. The assignment of a search target polarity of +1 to any non-palindromic search target (Qₙₚₐ) in the definition of (Gₚ) also specifies that in the search for ACP fragments of strand-polarity −1, only the negative target-string (S₋ⱼ) of the search target (Qₙₚₐ) is used to score an occurrence of (Qₙₚₐ), and occurrences of the positive target-string (Sⱼ) are ignored. The assignment of a search target polarity of −1 to any non-palindromic search target (Qₙₚₐ) in the definition of Gₚ specifies that in the search for ACP fragments of strand-polarity +1, only the negative target-string (S₋ⱼ) of the search target (Qₙₚₐ) is used to score an occurrence of (Qₙₚₐ), and occurrences of the positive target-string (Sⱼ) are ignored. The assignment of a search target polarity of −1 to any non-palindromic search target (Qₙₚₐ) in the definition of Gₚ also specifies that in the search for ACP fragments of strand-polarity −1, only the positive target-string (Sⱼ) of the search target (Qₙₚₐ) is used to score an occurrence of (Qₙₚₐ), and occurrences of the negative target-string (S₋ⱼ) are ignored.

During the search for process-pattern entities using the search target-group (Gu) with Mᵤ major classes, the search procedure of the present invention may specify that the only allowed polarity of the initial search step is left-to-right (+1) for ACP fragments with strand-polarity +1, and the only allowed polarity of the initial search step is right-to-left (−1) for ACP fragments with strand-polarity −1. The procedure may specify that both of the possible initial search step polarities (+1 and −1) are used for ACP fragments with strand-polarity zero. Regardless of the initial search step polarity (+1 or −1), each subsequent search step may be set up to proceed with the opposite search polarity of the previous search step. Furthermore, the procedure may specify that every search step with a left-to-right (+1) search polarity uses a furthest-right extremum condition, and every search step with a right-to-left (−1) search polarity uses a furthest-left extremum condition.

3) Presenting Results of SQF Analyses

As described above, a structured query fragment (SQF), obtained by the SQF analysis (U) using the dataset (Du) and the search target-group (Gu) as described in more general terms above, is defined as a substring within one of the resulting ACP fragments, where the SQF's termini are any two search target sites that are part of the set of Mᵤ sites in a process-pattern entity that can be derived from the ACP fragment. The definition of the search target-group (Gu) together with the process-pattern entity's definition are integral parts of the definition of the SQFs that can be derived there from. Therefore, display of SQF and/or process-pattern results in a table typically includes the display of a field (column) for a search target-group identifier (ID) and one, or more typically two, fields (columns) used to unambiguously identify the process-pattern (Examples Table 14).

The results of SQF analyses are stored in one or more results tables (FIG. 2D; see also Examples Table 14) in the database application of the present invention. These tables typically may include, as non-limiting examples, fields or combinations of fields for the following identifying information regarding the process-patterns detected for the analysis in question: an SQF analysis ID, a sequence ID, a target-group ID, an identifying (Qa) site of the ACP fragment, self-documenting process-pattern description, and SQF length data, typically in separate fields. Preferably, the results tables only include information regarding each of the two SQFs adjacent to the last search target site of the process-pattern entity. Process-pattern descriptions are preferably self-documenting, and typically consist of ordered numeric representations of the class permutation and member “permutation”, where the n-th digit of each number may preferably correspond to the class index and member index, respectively, of the search target site discovered in the n-th step of the search process. Although not a formal permutation, the member “permutation” is typically an inseparable contribution to the “class+member” permutation required to define each search target site in a process-pattern.

The database application of the present invention includes various stored procedures that execute database queries of the SQF analysis results tables mentioned above. These queries may provide comparative or summary information (Examples Tables 17-21) regarding process-patterns and SQFs of interest generated from a specified dataset using a specified search target-group, or comparative information regarding process-patterns and SQFs of interest generated from two specified datasets that had been analyzed using the same search target-group (Examples Table 16). The summary information queries may provide more detailed information regarding the SQFs of interest that were generated. For example, the output of summary queries may include information regarding the total number of SQFs of interest obtained in various general size ranges, typically including short, “ranged” (between a user-defined lower and upper limit), and long size ranges (Examples Tables 17-21).

II. Laboratory Identification of Structured Query Fragments

The laboratory embodiment of the present invention is a laboratory SQF analysis method for the identification, classification, comparison, generation, and separation of fragments derived from one or more physical samples of polydeoxyribonucleotides, including, but not limited to, physical samples of polydeoxyribonucleotides that are the reverse-transcription products of polyribonucleotides. Typically, the laboratory SQF analysis method faithfully emulates the computational SQF analyses described above.

The laboratory method is similar to the general searching process described above wherein:

a) the set of strings is a physical sample of polynucleotides;

b) the structured query fragments are physical polynucleotide fragments that are produced after the processing of the set of strings; and

c) the method typically further comprises detecting the physical polynucleotide fragments, or the use of the said fragments for various other analytical or preparative purposes.

Many laboratory embodiments are contemplated that fall within the present invention. These embodiments generally involve a hierarchical, recursive procedure that consists of a series of distinct, sequence-specific, double-stranded-cleavage reactions carried out on distinct fractions of DNA fragments that are immobilized, typically at one of their termini, using a separate, physically isolated solid support for each distinct fraction. For certain distinct fractions, the DNA fragments that are immobilized at their “proximal” termini (for each fragment, the terminus that is attached to the solid support) are also end-labeled at the termini that are distal to the solid support, where the attached label is a chemical moiety that can effect subsequent termini-specific immobilization (i.e., to a separate solid support) of any labeled, progeny DNA fragments that are liberated, by sequence-specific cleavage, from the parent fragments on the parent solid support. Unlabeled progeny DNA fragments that are liberated, by sequence-specific cleavage, from the parent fragments on the parent solid support cannot be re-immobilized and are not of interest.

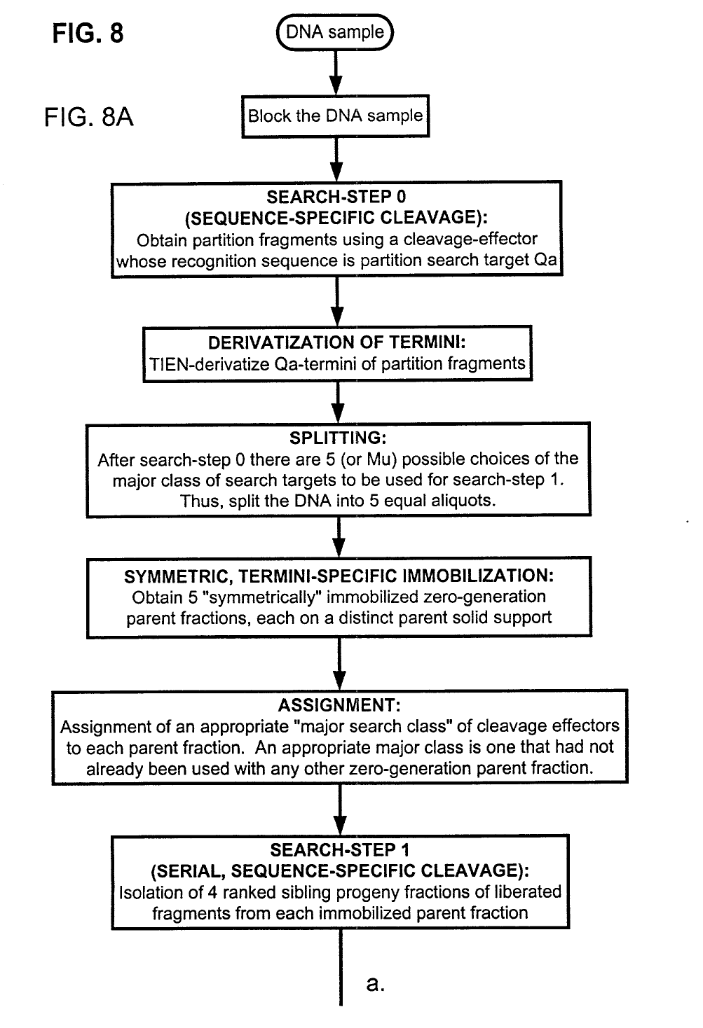

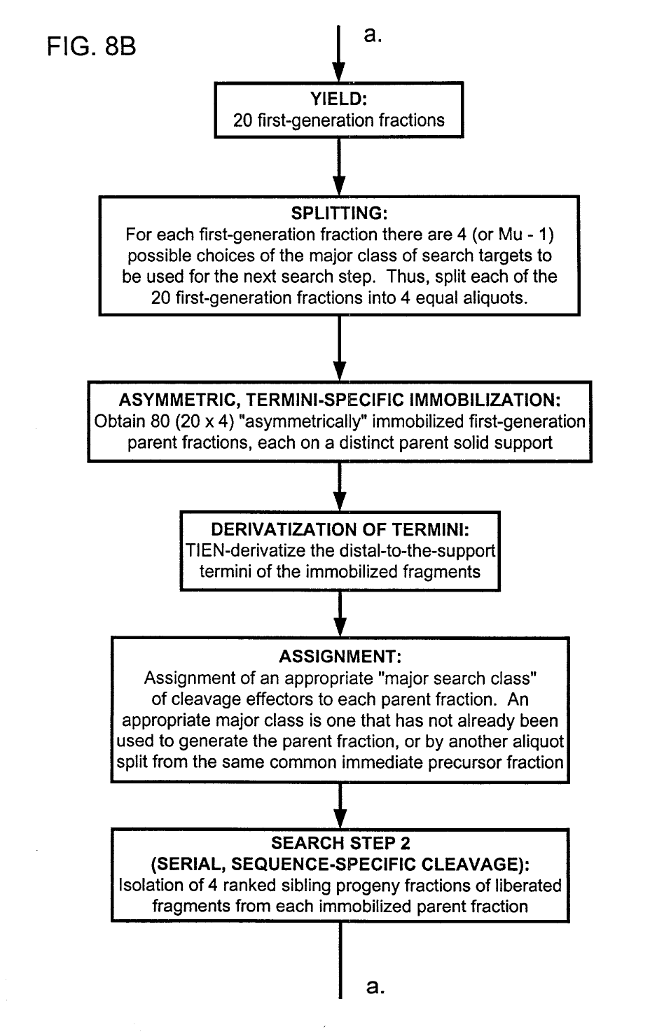

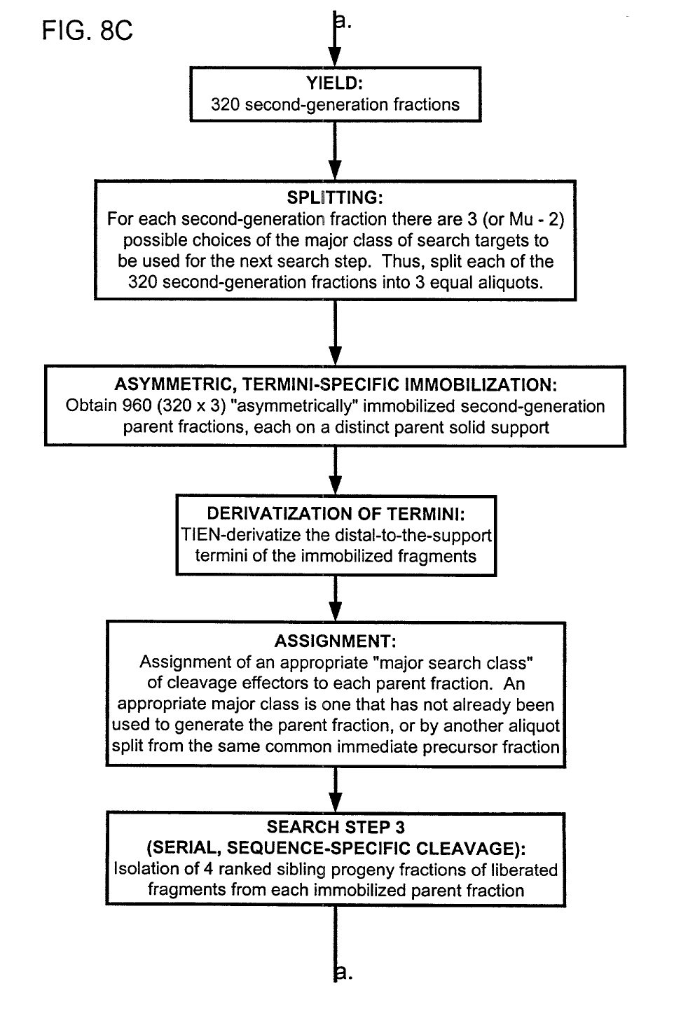

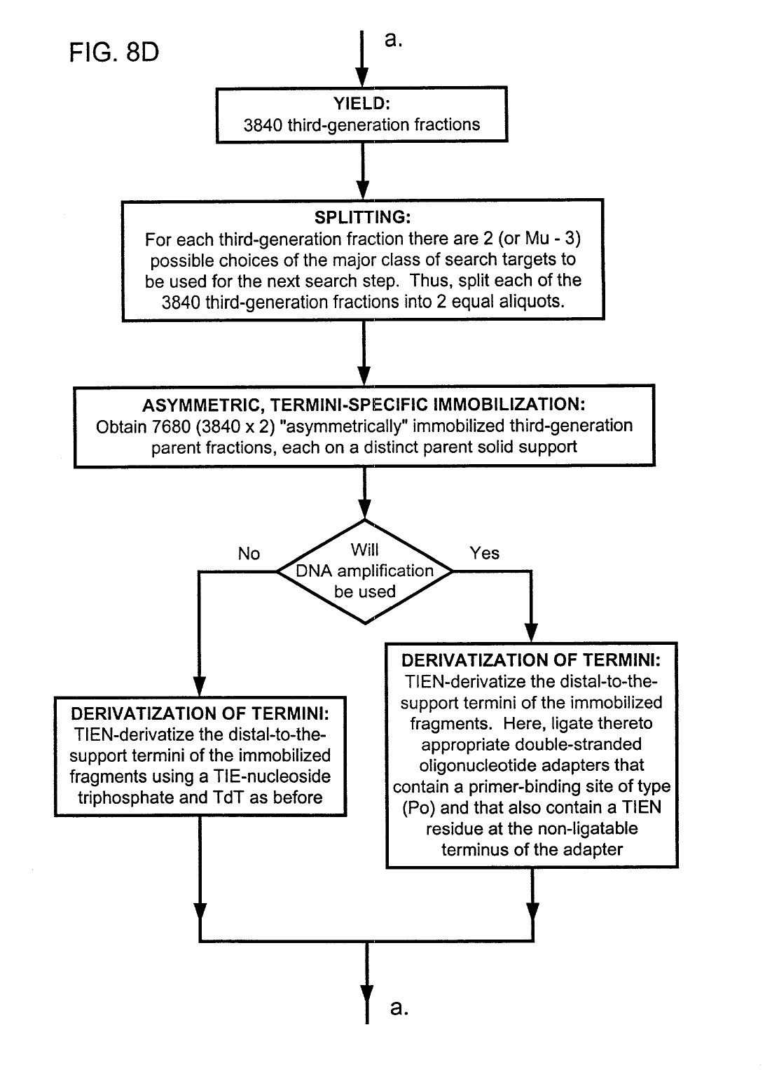

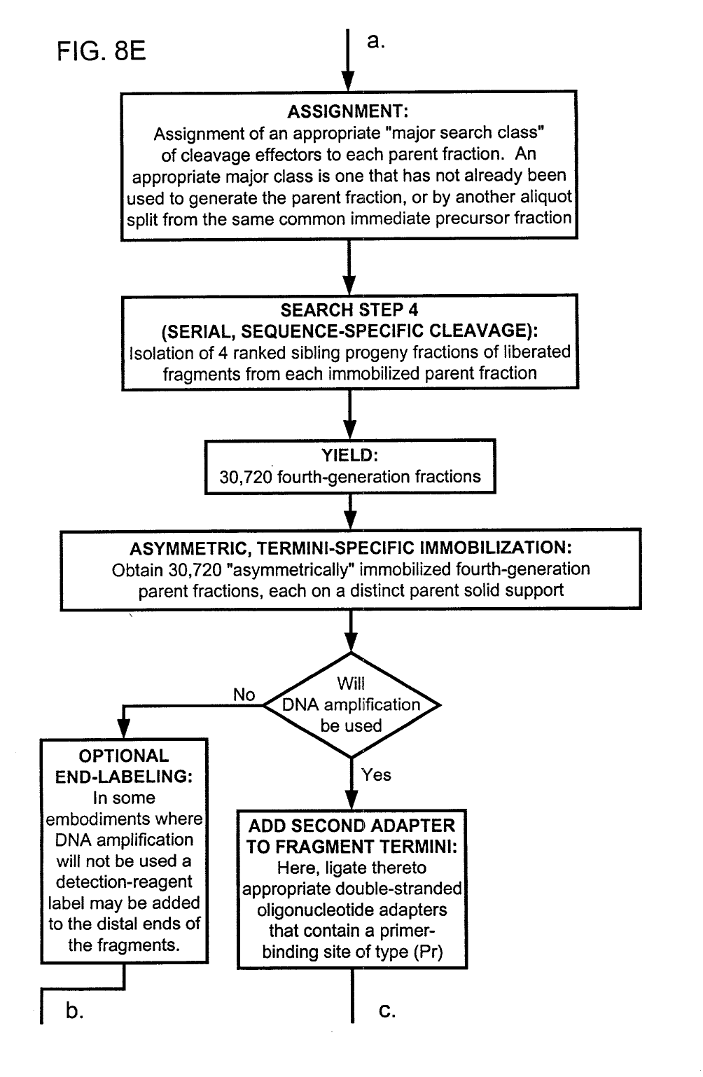

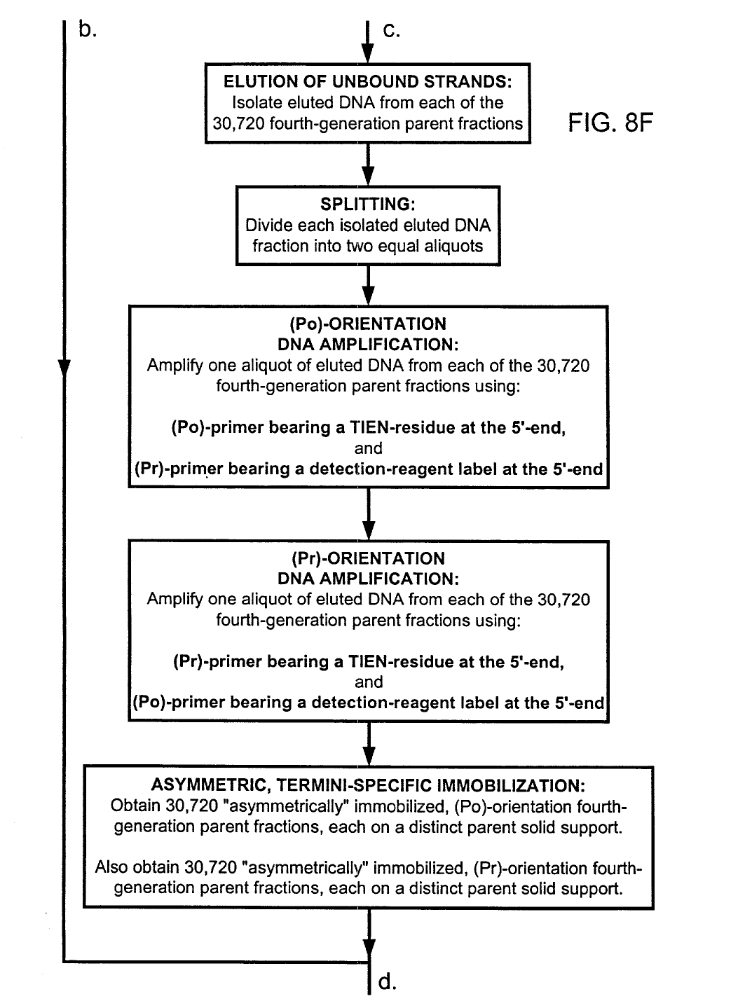

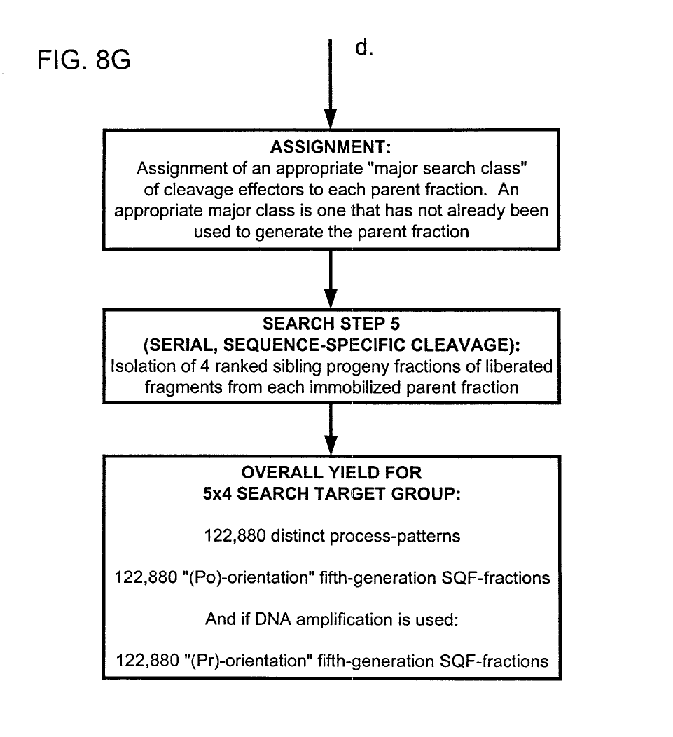

Thus, in certain steps of these embodiments, after a sequence-specific, double-stranded DNA cleavage event, the liberated, end-labeled fragments that were “most-distal” to (absolutely furthest from) the parent solid support may themselves be isolated as a specific progeny fraction (or more correctly, as the only meaningful component of a specific progeny fraction), and then re-immobilized, using their labeled termini, with opposite orientation on a new progeny solid support, and then end-labeled at the DNA fragment termini that are distal to the progeny solid support, and thus serve as a substrate for the next series of sequence-specific, double-stranded DNA cleavage reactions. Furthermore, in these embodiments the parent fragments that remain attached to the parent solid support after the cleavage step described above may themselves still serve as a substrate for another sequence-specific, double-stranded DNA cleavage reaction. Thus, in certain steps of these embodiments, each specific fraction of parent fragments immobilized on a distinct solid support may generate, in a serial fashion, several distinct fractions that contain end-labeled, “most-distal” sibling progeny fragments that may themselves be re-immobilized on distinct progeny solid supports, and where the distinct sibling progeny fractions are ranked fractions, based on the order in which they were generated by distinct, sequence-specific, double-stranded DNA cleavage reactions from a common parent fraction of fragments immobilized on a common parent solid support (FIGS. 8-10).

Ultimately in these embodiments, certain specific fractions of interest may be subject to various preparative or analytical procedures, including ligation-mediated DNA amplification using two types of generic, double-stranded oligonucleotide adapters and two types of corresponding amplification primers, as described below. In some of these embodiments the said DNA amplification reactions may also label the fragments present in specific fractions of interest, which may then be analyzed, typically to determine the number and size of the fragments therein, which in some specific fractions may include fragments that are individually distinguishable.

In a non-limiting example, such a laboratory embodiment is typically emulated by a corresponding computational embodiment, where the search target-group that is used is typically comprised of search targets that represent the recognition sequences of the enzymes or enzymatic processes, or equivalent chemical reagents or physical processes, that effect sequence-specific, double-stranded cleavage of DNA at, or a known distance from, their respective recognition sequences, and where the said set of sequence-specific, double-stranded DNA “cleavage effectors” are those used in the corresponding laboratory embodiment.

The sequence-specific cleavage and isolation of a given ranked sibling progeny fraction of DNA fragments from its immobilized parent fraction as described above in the laboratory embodiments of the present invention is thus conceptually equivalent to a massively parallel major-class-specific process-pattern search step as described in the computational embodiments of the present invention. This “physical” search step is massively parallel because it “searches” (cleaves in a sequence-specific manner) only those DNA fragments, and all such fragments, that are present in the immobilized parent fraction, and that: (i) contain an accessible recognition site for the sequence-specific cleavage effector used for the search step, and (ii) also lack any accessible recognition sites for the sequence-specific cleavage effectors used to generate any of the higher-ranked sibling progeny fractions from the same common parent fraction immobilized on the common parent solid support.

The following sections include a demonstrative subset of possible laboratory embodiments. However, other methods may be developed, using the principles of this application and well-known laboratory procedures, to achieve the present invention.

A) obtaining a physical sample or samples of polynucleotides

For the present invention, physical polynucleotide samples from any species can be used. The laboratory methods typically use one or more physical samples of polynucleotides, each of which is typically obtained from an individual. Preferably the polynucleotide sample is of sufficient purity (e.g., free of tissue-source or isolation-procedure-introduced contaminants), physical integrity (e.g., undegraded), and is substantially completely, preferably completely, dissolved in the sample solution in an appropriate buffer (one that will not affect the initial processing step).

One or more pooled samples of polynucleotides may also be used, where each pooled sample includes two or more distinct physical samples of polynucleotides, preferably DNA. Preferably, each sample that contributes to the pooled sample is obtained from a separate individual. Preferably, the DNA present in each of the contributing samples is of comparable purity and physical integrity, and is substantially completely, preferably completely, dissolved in its respective sample solution in an appropriate buffer (one that will not affect the initial processing step). Preferably, equal mass amounts of DNA are taken from each distinct contributing DNA sample to form the pooled sample.

B) Defining Recognition Site Process-Patterns Using Sequence-Specific, Double-Stranded Polynucleotide Cleavage Effectors

Typically, for the laboratory method of the present invention, each search target (Q) in Gu is the recognition sequence for a sequence-specific, double-stranded polynucleotide cleavage effector (Example Tables 3-5). The cleavage effector may be an enzyme or enzymatic process, a chemical reagent or chemical process, or a physical process, that can create a double-stranded cleavage point in a polydeoxyribonucleotide in a sequence-specific manner at, or a known distance from, the recognition sequence with which the enzyme, reagent, or process is associated. Such cleavage effectors are well known in the art, and a very large number of them are available from a variety of commercial sources.

The sequence-specific cleavage effectors used may be sequence-specific endonucleases, more particularly Type II restriction endonucleases, which are well-known in the art and are commercially available from Amersham Pharmacia Biotech Inc. USA (Piscataway, N.J., USA), New England BioLabs Inc. (Beverley, Mass., USA), Promega Corp. (Madison, Wis., USA), and Roche Molecular Biochemicals (Indianapolis, Ind., USA), among other suppliers. The sequence-specific cleavage effectors whose recognition sequences are used as major search targets may be restricted to those whose cleavage sites produce ligatable DNA fragment termini (caudemers) with either blunt ends or with single-stranded overhanging regions of length greater than one nucleotide. Type II restriction endonucleases that fulfill this “ligatable caudemer” requirement are well-known in the art and are commercially available from the suppliers of Type II restriction endonucleases mentioned earlier.

The sequence-specific cleavage effectors used may include cleavage effectors whose sequence-specific cleavage activity is un-inhibited or inhibited by the presence of naturally occurring modified nucleotide residues that may be present within, or immediately adjacent to, some or all of the DNA regions representing the cleavage effector's recognition sequence. As a non-limiting example, both cytosine-methylation-insensitive and cytosine-methylation-sensitive Type II restriction endonucleases may be used. Such enzymes are well-known in the art and commercially available from the suppliers of Type II restriction endonucleases mentioned earlier. A comprehensive listing of the known methylation sensitivities of restriction enzymes (http://rebase.neb.com/cgi-bin/mslist) is available online as part of REBASE, (http://rebase.neb.com/rebase/rebase.html), the restriction enzyme database that can be found at the website of New England Biolabs, Inc. (Beverley, Mass., USA).

C) Comprehensive-Scale Processing of a Polynucleotide Sample to Obtain Physical Structured Query Fragments

In the following description of a preferred laboratory embodiment of the present invention, a non-limiting example of a “5×4” search target-group (Gu) is used for the purposes of illustration, where Gu has Mᵤ=5 major-classes, with 4 search target members in each major class. The steps of a comprehensive-scale search strategy typically include those discussed in detail in the following paragraphs (FIG. 8).

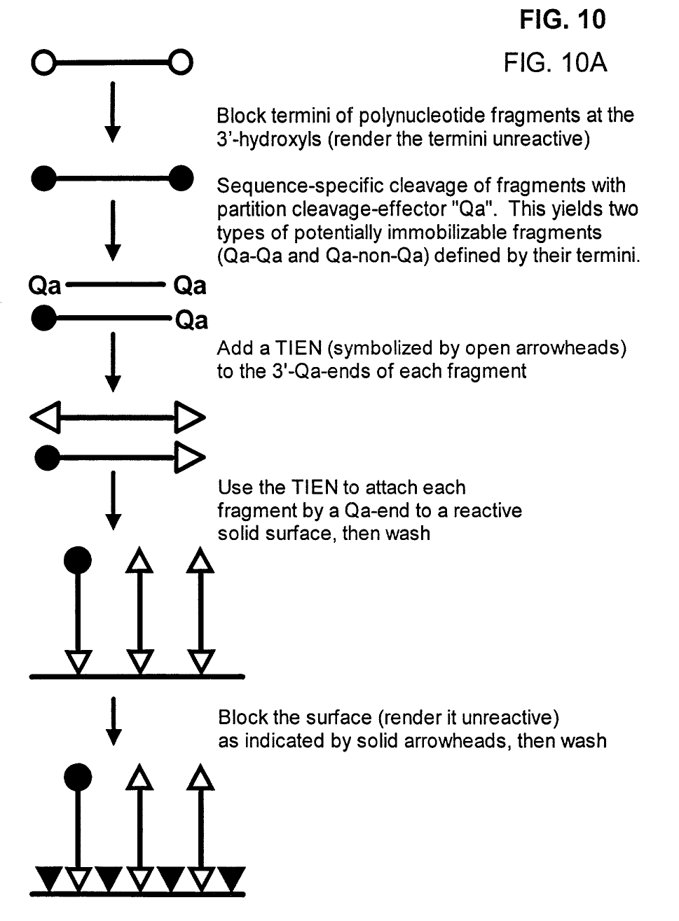

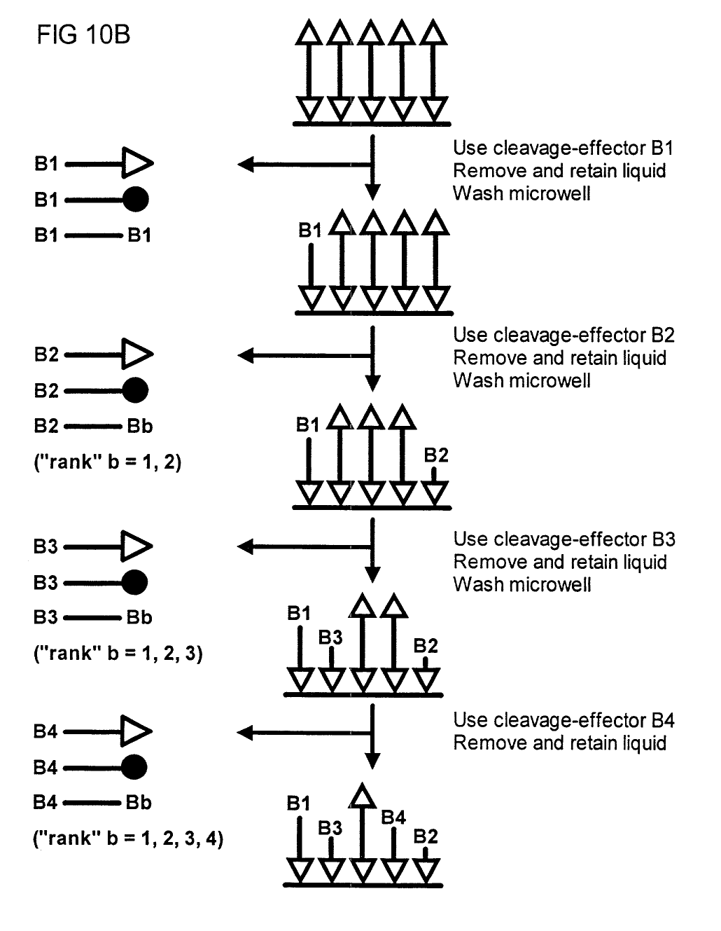

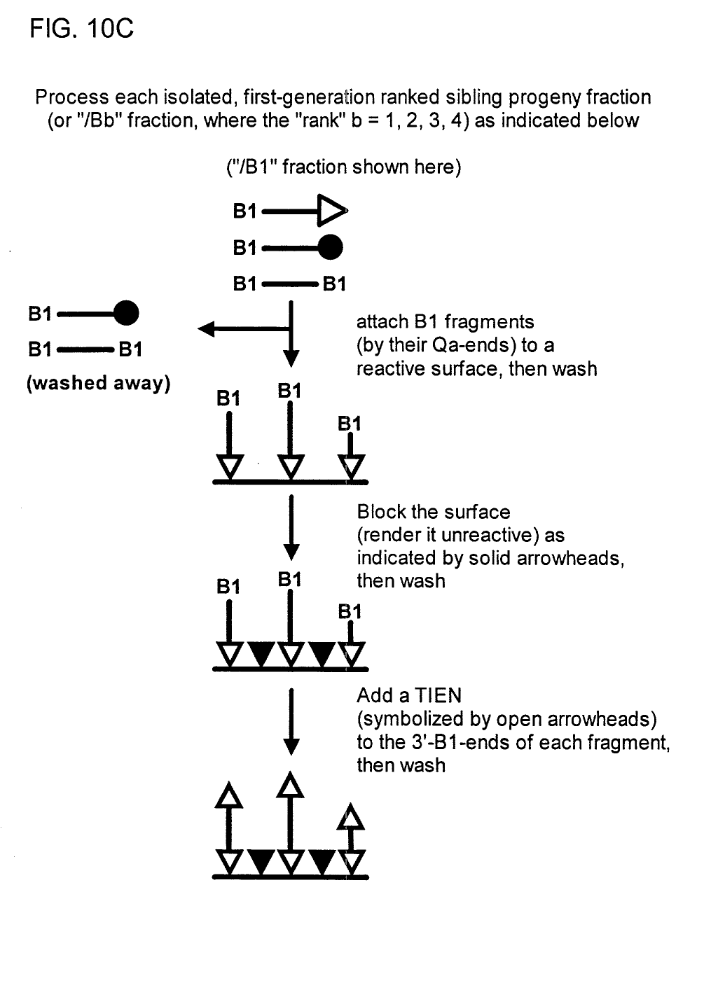

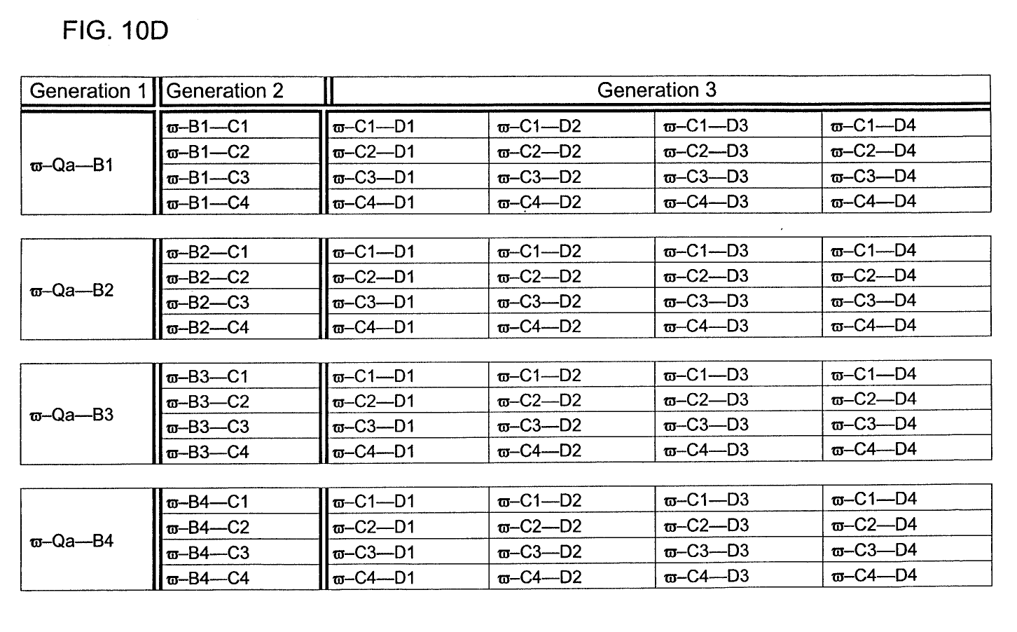

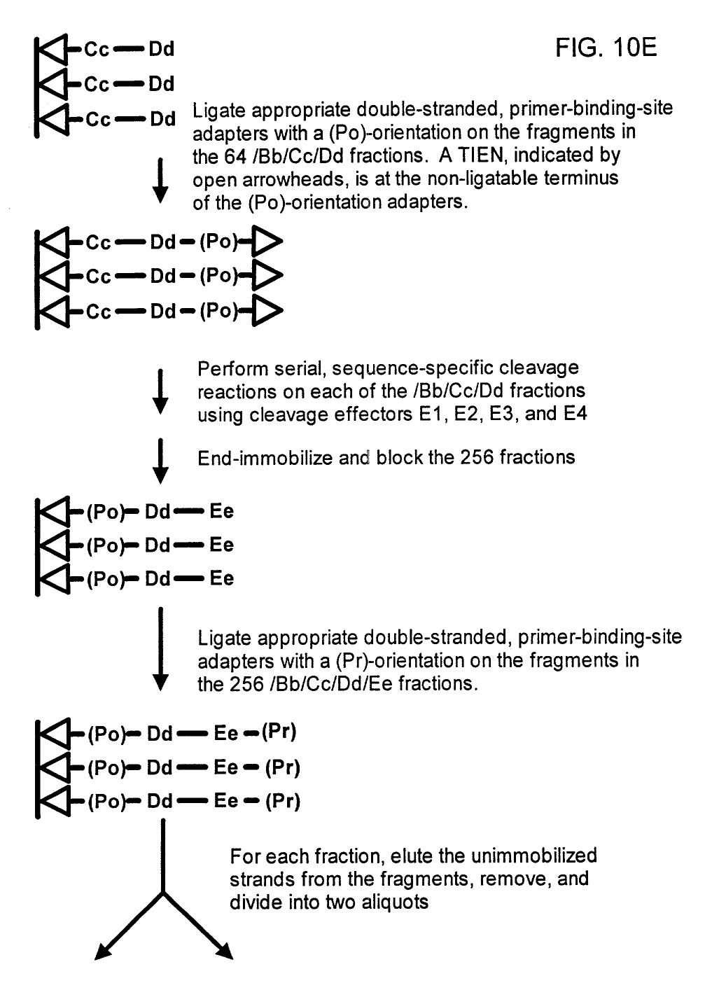

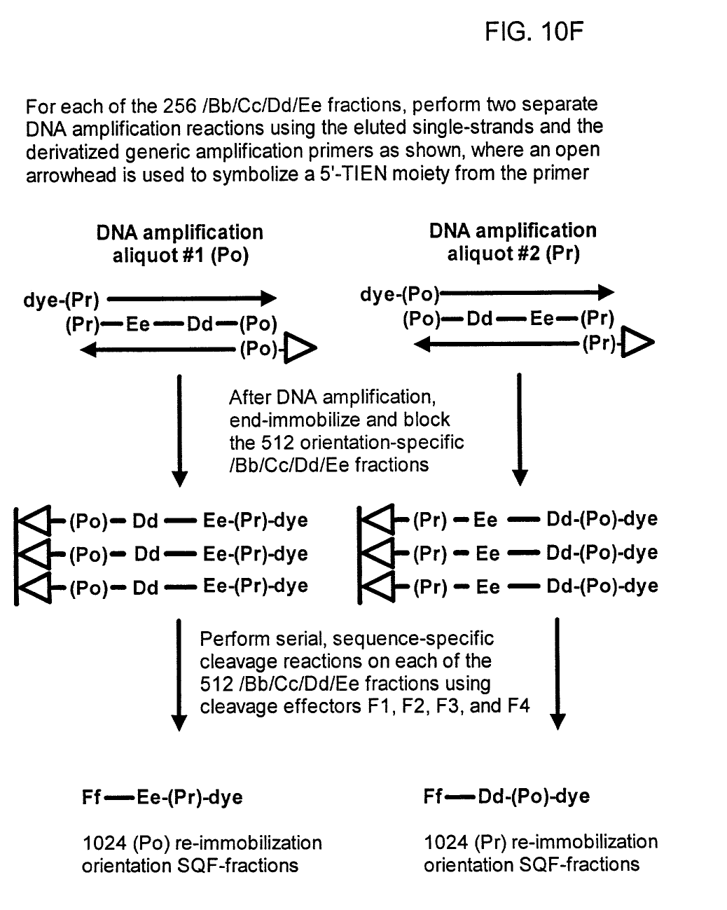

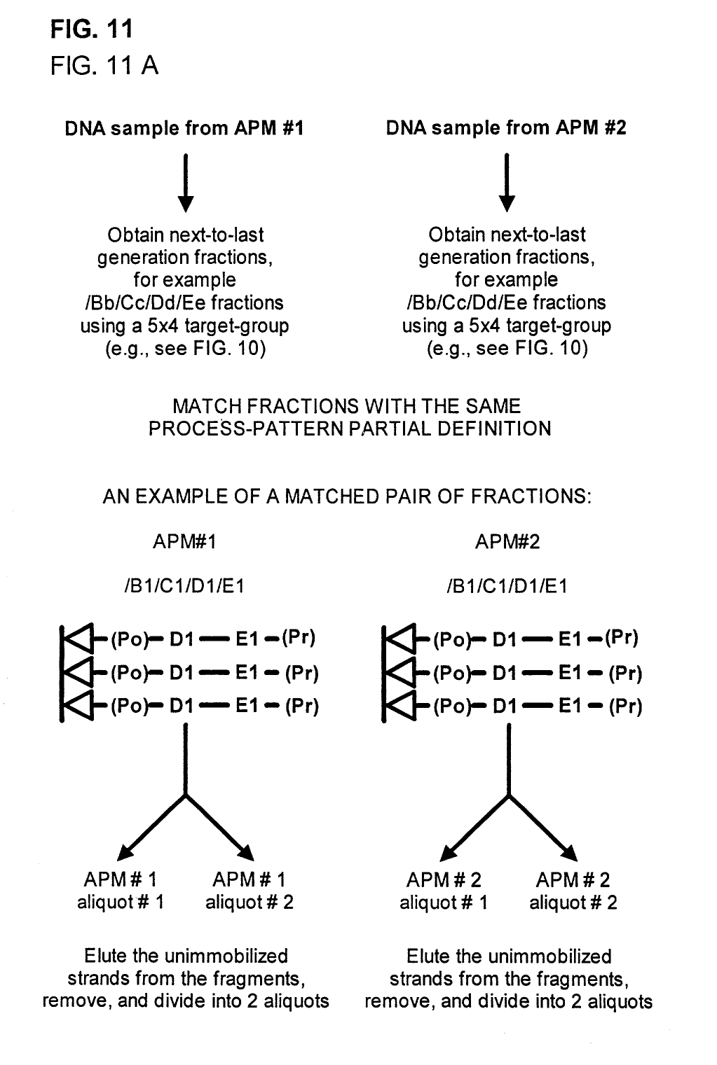

1) The procedure typically begins by the “blocking” (rendering unreactive for subsequent steps) of the 3′-hydroxyl groups at the termini of the fragments in the polynucleotide sample, preferably a DNA sample (see FIG. 8A; see also FIG. 10A). Many methods are known in the art for accomplishing this blocking step. As a non-limiting example, 3′-hydroxyl groups at the termini of DNA fragments may be blocked by the enzymatic incorporation of a mixture of dideoxynucleotides and α-thio deoxynucleotides, using one or more dideoxynucleoside triphosphates and one or more α-thio deoxynucleoside triphosphates (e.g., see Takada, S. et al. [1999] Genomics 61, 92-100), and the enzyme Terminal deoxynucleotidyl Transferase (symbolized as “TdT”, often simply referred to as “Terminal Transferase”). Terminal Transferase, dideoxynucleoside triphosphates, and a-thio deoxynucleoside triphosphates are all commercially available from Amersham Pharmacia Biotech Inc. USA (Piscataway, N.J., USA), among other suppliers.

2) Next, typically the polynucleotide sample, more typically the DNA sample, is completely digested using the sequence-specific, double-stranded-cleavage effector whose recognition sequence is the partition search target (Qa) as defined in (Gu). Methods are well-known in the art for performing restriction enzyme digestions of DNA such that the DNA is completely digested. The appropriate reaction conditions necessary to achieve complete, sequence-specific, double-stranded digestion of DNA are typically supplied by the vendor, in the form of printed instructions, whenever commercially available restriction enzyme products are purchased.

3) Next, unblocked groups at the termini of the polynucleotide fragments, preferably 3′-hydroxyl groups, are specifically activated (or derivatized) for subsequent immobilization. Specific activation of one terminus of a fragment renders the fragment capable of “asymmetric”, activated-terminus-specific immobilization to an appropriately derivatized solid support. The laboratory embodiment of the present invention makes the reasonable assumption, based on the known structural properties of DNA fragments in solution (i.e., that the flexibility of the DNA double-helix is typically limited in short, linear DNA fragments), that the specific activation of both termini of a single fragment, and subsequent reaction of such a fragment with an appropriately derivatized solid support, typically does not result in the activated-terminus-specific immobilization of the said fragment via both of its termini (“two-point attachment”). Instead, the said fragment more typically is capable of “symmetric”, activated-terminus-specific immobilization to an appropriately derivatized solid support with equal probability via one of either of its two termini. Even in those cases where “two-point attachment” is possible and does occur to a limited degree, the fragments so immobilized become an essentially inert component that does not compromise the laboratory embodiments of the present invention. Finally, a non-activated (or non-derivatized) terminus of a DNA fragment is, by definition, unable to effect activated-terminus-specific immobilization to the appropriately derivatized solid support.

Many methods for the specific activation (or derivatization) of DNA fragments for immobilization via their termini are known in the art and may be used with the present invention. Well-known, non-limiting examples include those that require, or effectively emulate, the incorporation of a specially modified “terminal-immobilization enabling (TIE) nucleotide” or (TIEN) at the free 3′-hydroxyl groups at the ends of fragments to be immobilized by their termini. The incorporation of a TIEN moiety at terminal 3′-hydroxyl groups typically requires the use of a TIE-nucleoside triphosphate. Non-limiting examples of these products include (i) 5-(3-aminoallyl)-2′-deoxyuridine-5′-triphosphate; (ii) 5-(N-[N-Biotinyl-epsilon-aminocaproyl]-3-aminoallyl)-2′-deoxyuridine 5′-triphosphate; (iii) 5-(N-[N-Biotinyl-epsilon-aminocaproyl-gamma-aminobutyryl]-3-aminoallyl)-2′-deoxyuridine 5′-triphosphate; and (iv) 5-(N-[N-Biotinyl-epsilon-aminocaproyl-gamma-aminobutyryl]-3-aminoallyl)-2′, 3′-dideoxyuridine 5′-triphosphate, where all of these products are commercially available from the Sigma Chemical Co. (St. Louis, Mo., USA), among other suppliers, and where these products will hereafter be referred to as examples of an “amino-TIEN”(i) or a “biotin-TIEN” (ii, iii, iv), respectively, because for these two types of TIEN, the reactive functional group attached to the nucleotide is either a primary amine or biotin, respectively.

It is well-known in the art that Terminal Transferase can catalyze the template-independent addition of deoxynucleoside triphosphates (or modified dNTP derivatives) at the terminal 3′positions of double-stranded DNA fragments bearing 3′- or 5′-overhangs or blunt-ended termini (see Kumar, A., et al. [1988] Anal. Biochem. 169, 376-382; and Schmitz, G. G., et al. [1991] Anal. Biochem. 192, 222-231). Typically, this enzyme is adopted for use in laboratory SQF analysis to incorporate modified deoxynucleoside triphosphates at the terminal 3′positions of double-stranded DNA. However, it is well-known in the art that there are other commercially available enzymes that may be used to accomplish the same objective.

In the description of subsequent steps, TIEN termini are indicated only for descriptive purposes. Any equivalent specific activation (or derivatization) of DNA fragment termini for immobilization on an appropriately derivatized solid support could be utilized in these steps. Typically, “biotin-TIEN-” or “amino-TIEN-” labeled DNA fragments are immobilized either on streptavidin-coated solid supports or N-hydroxysuccinimide-derivatized solid supports, respectively. Typically, large numbers of distinct solid supports are conveniently available as individual derivatized microwells of a 96-well disposable microplate, where the said disposable microplates can be used at the typical reaction temperatures required for the sequence-specific, double-stranded DNA cleavage effectors used. Both streptavidin-coated or N-hydroxysuccinimide-derivatized 96-well disposable microplates that would be suitable, given the criteria mentioned above, are commercially available from Corning Inc. Life Sciences (Acton, Mass., USA), among other suppliers.

Typically, the “comprehensive-scale” preferred laboratory embodiment of the present invention involves processing the 96-well disposable microplates using programmable, automated laboratory liquid-handling and plate-management equipment. Such equipment is commercially available from Beckman Coulter, Inc. (Fullerton, Calif., USA) and Zymark Corporation (Hopkinton, Mass., USA), among other suppliers.

4) The resulting solution of derivatized (i.e., specifically activated) partition fragments, which typically includes symmetrically derivatized (TIEN-Qa-DNA-Qa-TIEN) and asymmetrically derivatized (TIEN-Qa-DNA-dideoxynucleotide) partition fragments, is obtained in a buffer that is suitable for activated-terminus-specific immobilization of the partition fragments to appropriately derivatized solid supports. The solution is then divided into Mᵤ equal aliquots (e.g., in this non-limiting example) that represent distinct “zero-generation” parent fractions (FIG. 8A). Each of these parent fractions is then immobilized on a distinct, appropriately derivatized parent solid support using immobilization techniques described above. The only productively immobilized partition fragments on each of the “zero-generation” parent solid supports are symmetrically derivatized partition fragments (TIEN-Qa-DNA-Qa-TIEN), where the said partition fragments may be immobilized, with equal probability, at one of either of their ends, and thus with either polarity. All subsequent TIEN-mediated immobilization reactions are by definition asymmetric. “Productively immobilized” means that fragments are immobilized in such a manner that specific, liberated derivatives that may be obtained thereof may themselves be immobilized in subsequent steps according to the laboratory procedures described herein.

(i) After “blocking” of the unreacted functional groups on the parent solid supports, as described above, and after an appropriate washing step, each of the 5 “zero-generation” parent fractions are assigned an appropriate major search class that in each case would be used to isolate four “first-generation” ranked sibling progeny fractions. An appropriate major search class for a “zero-generation” parent fraction PFx_{z} is one that had not already been used with any other “zero-generation” parent fraction PFx_{y}. The only meaningful component in the “first-generation” ranked sibling progeny fractions so obtained is the TIEN-labeled fragments that were previously “most-distal” to (absolutely furthest from), and subsequently liberated by sequence-specific cleavage from, their respective parent solid support.

Thus, for example, one of the “zero generation” parent fractions, which shall be referred to here as “g0_B”, is used as a solid-phase substrate for a reaction, using an appropriate reaction buffer, temperature, duration, etc., with the sequence-specific, double-stranded cleavage effector whose recognition sequence is the highest-ranked member (“B1”) of the specific major search class “B”. Upon completion of the reaction using “B1”, an appropriate stop solution is added, and the solution phase is isolated (i.e., transferred to a new storage microwell). This storage microwell contains the highest-ranked “first-generation” sibling progeny fraction obtained from g0_B. The said “zero-generation” parent fraction (g0_B) on the parent solid support which is used as a substrate for B1 as described above is then washed using an appropriate buffer solution, and used again as a solid-phase substrate for a distinct cleavage reaction, using an appropriate reaction buffer, temperature, duration, etc., with the sequence-specific, double-stranded cleavage effector whose recognition sequence is the second-highest-ranked member (“B2”) of the specific major search class “B”. Stoppage of this cleavage reaction and isolation of the second-highest ranked “first-generation” sibling progeny fraction from g0₁₃ B is as described hereinabove. Thus, in this manner, four “first-generation” ranked sibling progeny fractions (/B1, /B2, /B3, /B4) would be obtained in a serial fashion from the said “zero-generation” parent fraction (g0₁₃ B), and isolated into physically separate storage microwells (for example, see FIG. 10B).

In some preferred embodiments, one may typically need or prefer to purify individually the isolated, stopped cleavage reactions in these and other ranked sibling progeny fractions before subsequent processing steps. This “purification” step is typically more accurately described as a buffer exchange step, and may be required to prevent buffer components from the stopped cleavage reaction mixture from inhibiting subsequent steps (e.g., activated-terminus-specific immobilization of DNA fragments). This “purification” step may be effected using well known techniques and equipment, for example by using disposable, 96-well microplate form-factor units that are commercially available from Qiagen, Inc. USA (Valencia, Calif., USA) and Millipore Corp. (Bedford, Mass., USA), among other suppliers, and are typically compatible with the programmable, automated liquid-handling equipment and 96-well microplate handling equipment mentioned hereinabove.

One can describe progeny fractions of any “generation” in a manner similar to the hierarchical file system used by most modern computer operating systems. In one such notational system, the first index value (i) for a major search target Q_{i,j} denotes the major search class used in a given search step, and the second index value (j)denotes the ranked member of the indicated major search class. In an equivalent notational system, ascending alphabetic characters (typically B, C, D, E, and F) are used to denote the major search classes used, whilst a letter immediately following any of the said characters denotes the ranked member of the indicated major search class. In both notational systems, the ordered, major-class-specific search steps (or “generations”) may be separated by a forward-slash character (“/”) and are ordered from left to right in their natural order of execution; the individual “fraction lineages” may be separated by semi-colons. Thus the 20 “first-generation” progeny fractions can be described using either of the notational systems shown in Examples Table 12.

(ii) Then each of the 20 “first-generation” progeny fractions isolated as described above is divided into four (i.e., Mᵤ−1) equal aliquots and “asymmetrically” re-immobilized (via a TIEN-moiety-bearing terminus only), each on a distinct appropriately derivatized solid support, followed by blocking of unreacted functional groups on the said solid supports as described above, and an appropriate washing step as described above, to form 80 “first-generation” parent fractions (FIG. 8B). The reason that four (i.e., Mᵤ−1=4 here) equal aliquots are used is because one of the major search classes has already been used for the first search step, and thus for each of the 20 “first-generation” progeny fractions there are only four (i.e., Mᵤ−1=4 here) major search classes available to define the next search step that can be used to obtain all of the descendant progeny fractions (and define all of the process-patterns) that may be derivable from them. Thus, for each of the fractions (/Q_{1,1};/ Q_{1,2}; /Q_{1,3}; /Q_{1,4})=(/B1; /B2; /B3; /B4), only the major search classes numbered (2, 3, 4, or 5), or in an alternative notation, major search classes (C, D, E, and F), may be used to define the next search step that can be used to obtain all of the descendant progeny fractions (and define all of the process-patterns) that may be derivable from (/Q,_{1,1}; /Q_{1,2}; /Q_{1,3}; /Q_{1,4}).

It should be apparent that as was the case with the computational preferred embodiments of the present invention, combinatorics is very important to the power of the corresponding laboratory preferred embodiments of the present invention. With each generation “x” of progeny fractions, there are M^{u}−x possible major search classes of sequence-specific, double-stranded cleavage-effectors that can be used to obtain the next generation's ranked sibling progeny fractions using the search target-group (Gu) comprised of Mᵤ major search classes. The full pursuit of this combinatorial strategy will ultimately define all of the process-patterns that may be obtained from (Gu), and further obtain all of the SQFs of interest that are typically obtained using these process-patterns.

(iii) The immobilized DNA fragments in each of the 80 “first-generation”parent fractions are then TIEN-derivatized at their distal-to-the-solid-support, 3′-hydroxyl groups, using a TIE-nucleoside triphosphate and TdT as described above. At the conclusion of the derivatization reaction, the DNA fragments on the solid supports are washed using an appropriate buffer solution, as described above.

(iv) Each of the 80 “first-generation” parent fractions are then assigned an appropriate major search class that in each case is used to isolate four “second-generation” ranked sibling progeny fractions. The fractions are generated using the cleavage reaction and product isolation steps described earlier in general terms. An appropriate major search class for the processing of a “first-generation” parent fraction PFx_{z}, is one that: had not already been used in any prior search step used to produce PFx_{z}; and had not already been used with any other “first-generation” parent fraction PFx_{y} where PFx_{y} and PFx_{z} were produced using aliquots from the same “first-generation” progeny fraction. The only meaningful component in the “second-generation” progeny fractions so obtained is the TIEN-labeled fragments that were previously “most-distal” to (absolutely furthest from), and subsequently liberated by sequence-specific cleavage from, the parent solid support.

(v) Then each of the 320 “second-generation” progeny fractions so isolated is divided into three (i.e., Mᵤ−2) equal aliquots and “asymmetrically” re-immobilized (via a TIEN-moiety-bearing terminus only), each on a distinct appropriately derivatized distinct solid support, followed by blocking of unreacted functional groups on the said solid supports and an appropriate washing step as described above, to form 960 “second-generation” parent fractions (FIG. 8C). The fractions are generated using the cleavage reaction and product isolation steps described earlier in general terms. The reason that three (i.e., Mᵤ−2=3 here) equal aliquots are used is because two of the major search classes have already been used for the first two search steps, and thus for each of the 320 “second-generation” progeny fractions there are only three (i.e., Mᵤ−2=3 here) major search classes available to define the next search step that can be used to obtain all of the descendant progeny fractions (and define all of the process-patterns) that may be derivable from them.

(vi) The immobilized DNA fragments in each of the 960 “second-generation” parent fractions are then TIEN-derivatized at their distal-to-the-solid-support, 3′-hydroxyl groups, using a TIE-nucleoside triphosphate and TdT as described earlier. At the conclusion of the derivatization reaction, the DNA fragments on the solid supports are washed using an appropriate buffer solution.

(vii) Each of the 960 “second-generation” parent fractions are then be assigned an appropriate major search class that in each case would be used to isolate four “third-generation” ranked sibling progeny fractions. The fractions are generated using the cleavage reaction and product isolation steps described earlier in general terms. An appropriate major search class for the processing of a “second-generation” parent fraction PFx_{z} is one that: had not already been used in any prior search step used to produce PFx_{z}; and had not already been used with any other “second-generation” parent fraction PFx_{y} where PFx_{y} and PFx_{z} were produced using aliquots from the same “second-generation” progeny fraction. The only meaningful component in the “third-generation” progeny fractions so obtained is the TIEN-labeled fragments that were previously “most-distal” to (absolutely furthest from), and subsequently liberated by sequence-specific cleavage from, the parent solid support.

(viii) Then each of the 3840 “third-generation” progeny fractions so isolated is divided into two (i.e., Mᵤ−3) equal aliquots and “asymmetrically”re-immobilized (i.e., via a TIEN-moiety-bearing terminus only), each on a distinct appropriately derivatized solid support, followed by blocking of unreacted functional groups on the said solid supports and an appropriate washing step, to form 7680 “third-generation” parent fractions (FIG. 8D). The reason that (M^{u}−3=2 here) equal aliquots are used is because three of the major search classes have already been used for the first three search steps, and thus for each of the 3840 “third-generation” progeny fractions there are only (M^{u}−3=2 here) major search classes available to define the next search step that can be used to obtain all of the descendant progeny fractions (and define all of the process-patterns) that may be derivable from them.

(ix) The immobilized DNA fragments in each of the 7680 “third-generation” parent fractions are then derivatized at their distal-to-the-solid-support, 3′-hydroxyl groups. At this juncture, various laboratory embodiments may carry out this derivatization step in different ways (FIG. 8D). In some laboratory embodiments, referred to herein as “conventional derivatization reactions,” there may be no requirement to begin, at the present step, to enable the use of DNA amplification at a later step. Typically in such laboratory embodiments the detection of SQFs during later steps may be so sensitive, or there may be no interest in the eventual preparative production of SQFs, that DNA amplification during the course of SQF production is not needed. Thus, in such laboratory embodiments, each of the 7680 “third-generation” parent fractions would be derivatized at their distal-to-the-solid-support, 3′-hydroxyl groups using a TIE-nucleoside triphosphate and TdT as described earlier. At the conclusion of the conventional derivatization reactions, the DNA fragments on the solid supports are washed using an appropriate buffer solution.

More typically, derivatization is effected here by the ligation, using an appropriate DNA ligase, of a double-stranded, primer-binding-site containing oligonucleotide adapter to the distal end of the currently immobilized DNA fragments. A TIEN residue or the equivalent must be present at, and typically at the 5′-position of, the double-stranded oligonucleotide adapter's non-ligatable terminus. For each of the immobilized fractions, the double-stranded oligonucleotide adapter's ligatable terminus must be capable of a ligation reaction with the immobilized DNA fragments'distal caudemers (i.e., overhanging, single-stranded DNA tails, if any, generated by the previous sequence-specific cleavage step) by the presence at the oligonucleotide adapter's ligatable terminus of a 5′-phopshate group, and either a blunt-end where required, or the appropriate caudemers.

The double-stranded oligonucleotide adapter used is typically one of two possible types of such oligonucleotide adapter that are required for subsequent DNA amplification. The types of oligonucleotide adapter are distinguishable by the sequence of the adapter-core sequence (ACS) present therein. In one oligonucleotide adapter type the 3′-end of the ACS is directed towards the double-stranded adapter's ligatable terminus and the ACS is identical to the sequence of an “original-orientation” generic primer (Pₒ) used in a subsequent DNA amplification step. For the other type of oligonucleotide adapter, the 3′-end of the ACS is also directed towards the double-stranded adapter's ligatable terminus, but here the ACS is identical to the sequence of a “reverse-orientation” generic primer (Pᵣ) used in a subsequent DNA amplification step. Both of these two distinct, generic primer sequences (Pₒ and Pᵣ), when used together, have the property of being unable to produce detectable polymerase chain reaction (PCR) products, or equivalent products generated by an equivalent primer-dependent polynucleotide amplification technique, when used as a primer pair with DNA from the same source as that of the sample used in the current SQF analysis. Thus, naturally-occurring (Pₒ and Pᵣ) primer-binding sites within the DNA fragments to be amplified are typically absent.

If (y) equals the total number of major search targets in Gu (i.e., that represent recognition sequences for the sequence-specific cleavage effectors) that generate distinct caudemers (including the blunt-end case), then a maximum of (y) double-stranded oligonucleotide adapters, all of which contain the adapter-core sequence (Pₒ) may needed. Additionally, a maximum of another (y) double-stranded oligonucleotide adapters, all of which contain the adapter-core sequence (Pᵣ) may also needed. When DNA amplification is required, these two types (Pₒ and Pᵣ) of double-stranded oligonucleotide adapters must be available for each of the possible caudemers that may be generated by the sequence-specific cleavage effectors whose recognition sequences comprise the major search targets in Gu. This is required so that all possible orientation-specific, caudemer-specific, ligation-mediated derivatization reactions may be effected, and thus all possible process-pattern definitions may be obtained, and so too all of the SQFs of interest derived from them.

Thus, in those laboratory embodiments where DNA amplification is to be used at a later step, and where the generation (x)=Mᵤ−2=3 here, each of the 7680 “third-generation” parent fractions would be derivatized at their fragments' distal-to-the-solid-support, ligatable caudemers by the ligation thereto of the appropriate (ligation-capable) double-stranded, primer-binding-site containing oligonucleotide adapter. This adapter would have the (Pₒ) adapter-core sequence, and would also have a (TIEN) residue or the equivalent at the 5′-position of its non-ligatable terminus. At the conclusion of the ligation reaction, the DNA fragments on the solid supports would be washed using an appropriate buffer solution.

(x) Each of the 7680 “third-generation” parent fractions would then be assigned an appropriate major search class that in each case would be used to isolate four “fourth-generation” ranked sibling progeny fractions (FIG. 8E).

The fractions are generated using the cleavage reaction and product isolation steps described earlier in general terms. An appropriate major search class for the processing of a “third-generation” parent fraction PFx_{z} is one that: had not already been used in any prior search step used to produce PFx_{z}; and had not already been used with any other “third-generation” parent fraction PFx_{y} where PFx_{y} and PFx_{z} were produced using aliquots from the same “third-generation” progeny fraction. The only meaningful component in the “fourth-generation” progeny fractions so obtained is the TIEN-labeled fragments that were previously “most-distal” to (absolutely furthest from), and subsequently liberated by sequence-specific cleavage from, the parent solid support.

(xi) Then each of the 30,720 “fourth-generation” progeny fractions so isolated is “asymmetrically” immobilized (via a TIEN-moiety-bearing terminus only), each on a distinct appropriately derivatized solid support, followed by blocking of unreacted functional groups on the said solid supports and an appropriate washing step, to form 30,720 “fourth-generation” parent fractions. Four of the major search classes have already been used for the first four search steps, and thus each of the 30,720 “fourth-generation” progeny fractions is used as a single “fourth-generation” parent fraction, as there is only (Mᵤ−4=1 here) major search class available to define the next search step that can be used to obtain all of the progeny fractions (and complete the definition of all of the process-patterns) that may be derivable from them.

In some of the embodiments where DNA amplification had not been used, a detection reagent label may be attached to the distal ends of the fragments in each of the 30,720 “fourth-generation” parent fractions (FIG. 8E). Many types of detection reagent labels are known in the art. For example, but not intended to be limiting, the detection reagent may be a modified nucleoside triphosphate and TdT. Typically, addition of the detection reagent is followed by an appropriate wash step.

(xii) In those laboratory embodiments where DNA amplification is to be used at a later step, each of the 30,720 “fourth-generation” parent fractions would be derivatized at their fragments' distal-to-the-solid-support, ligatable caudemers by the ligation thereto of the appropriate (ligation-capable) double-stranded, primer-binding-site containing oligonucleotide adapter. This adapter would have the (Pᵣ) adapter-core sequence, and would have an underivatized non-ligatable terminus. At the conclusion of the ligation reaction, the DNA fragments on the solid supports would be washed using an appropriate buffer solution.

(xiii) In those laboratory embodiments where DNA amplification is to be used, the unbound strands of the immobilized fragments in each of the 30,720 “fourth-generation” parent fractions are eluted using denaturing reagents, elevated temperature, or both (FIG. 8F). Nucleotide denaturation reagents are well known in the art. Denaturing reagents are then removed and the resulting material is obtained in an appropriate buffer solution and divided into two equal aliquots. DNA fragments in each of the aliquots are then amplified using separate PCR reactions, or equivalent primer-dependent polynucleotide amplification reactions of which many are known in the art. Amplification of the first aliquot obtained above uses a (Pₒ) primer bearing a (TIEN) residue or the equivalent at the 5′-position, and typically uses a (Pᵣ) primer bearing a detection-reagent label (such as, but not limited to, one of the commonly used fluorescent sequencing labels) at the 5′-position. Amplification of the second aliquot obtained above uses a (Pᵣ) primer bearing a (TIEN) residue or the equivalent at the 5′-position, and typically uses a (Pₒ) primer bearing a detection-reagent label (as described above) at the 5′-position. Note that in some laboratory embodiments the use of detection-reagent labels may either not be required for subsequent analytical purposes, or be deliberately omitted for subsequent analytical or preparative purposes.

(xiv) In those laboratory embodiments where DNA amplification had been used as described above, the products of each amplification reaction are terminally immobilized (or “re-immobilized”) via their TIEN-containing fragment termini onto distinct, appropriately derivatized solid supports, followed by blocking and washing steps as described earlier. Thus, each of the 30,720 “fourth-generation” parent fractions yields two distinct “re-immobilization-orientation-specific, fourth-generation” parent fractions on distinct parent solid supports.

(xv) In those laboratory embodiments where DNA amplification had been used as described above, each of the 61,440 “re-immobilization-orientation-specific, fourth-generation” parent fractions are then assigned the appropriate major search class that in each case would be used to isolate four “re-immobilization-orientation-specific, fifth-generation” ranked sibling progeny fractions. The fractions are generated using the cleavage reaction and product isolation steps described earlier in general terms. The appropriate major search class for the processing of a “re-immobilization-orientation-specific, fourth-generation” parent fraction PFx_{z} is the only remaining major search class that has not already been used in any prior search step used to produce PFx_{z}. The only meaningful component in the “re-immobilization-orientation-specific, fifth-generation” progeny fractions so obtained is the detection-reagent-labeled fragments that were previously “most-distal” to (absolutely furthest from), and subsequently liberated by sequence-specific cleavage from, the parent solid support.

In those laboratory embodiments where DNA amplification is not used, there are only 30,720 “fourth-generation” parent fractions available for this step. Otherwise, the generation of the “fifth-generation” ranked sibling progeny fractions here using the final major search class is as described immediately above.

(xvi) In those laboratory embodiments where DNA amplification had been used as described above, each of the 122,880 distinct process-patterns that are obtainable from a 5×4 search target-group yield two “re-immobilization-orientation-specific, fifth-generation” ranked sibling progeny fractions (FIG. 8G). Thus, a total of 245,760 “re-immobilization-orientation-specific, fifth-generation” ranked sibling progeny fractions typically may be obtained after “comprehensive-scale” processing of a polynucleotide sample using a 5×4 search target group. To reduce verbiage, the “last-generation”ranked sibling progeny fractions obtained using embodiments of the present invention are often simply referred to as “SQF-fractions”.

In those laboratory embodiments where DNA amplification had been used as described above, half of the 245,760 SQF-fractions so obtained, or 122,880 SQF-fractions, were obtained using the “original” {TIEN-(Pₒ)-adapter-mediated} immobilization orientation of the “fourth-generation” (or “next-to-last-generation”) parent fractions. Found within each of these 122,880 SQF-fractions are all of the SQFs that may be obtained using either of the possible initial search step polarities (i.e., either of the possible partition fragment initial immobilization polarities), in all of the ACP (i.e., partition) fragments that may be obtained from the entire polynucleotide starting material, and where the said SQFs are those between the search target sites defined by steps (Mᵤ) and (Mᵤ−1) of the process-pattern and the re-immobilization orientation used to generate each SQF-fraction.

In those laboratory embodiments where DNA amplification had been used as described above, half of the 245,760 SQF-fractions so obtained, or 122,880 SQF-fractions, are obtained using the “reverse” {TIEN-(Pᵣ)-adapter-mediated} immobilization orientation of the “fourth-generation” (or “next-to-last-generation”) parent fractions. Found within each of these 122,880 SQF-fractions are all of the SQFs that may be obtained, using either of the possible initial search step polarities (i.e., either of the possible partition fragment initial immobilization polarities), in all of the ACP (partition) fragments that may be obtained from the entire polynucleotide starting material, and where the said SQFs are those between the search target sites defined by steps (Mᵤ) and (Mᵤ−2) of the process-pattern and the re-immobilization orientation used to generate each SQF-fraction.

In those laboratory embodiments where DNA amplification had not been used, there would only be 122,880 “fifth-generation” ranked sibling progeny fractions (SQF-fractions), each of which was obtained using one of the 122,880 distinct process-patterns that are obtainable from a 5×4 search target-group. These 122,880 SQF-fractions contain SQFs of the “original” immobilization orientation used to produce the fourth-generation (or “next-to-last-generation”) parent fractions. Found within each of these 122,880 SQF-fractions are all of the SQFs that may be obtained, using either of the possible initial search step polarities (i.e., either of the possible partition fragment initial immobilization polarities), in all of the ACP (partition) fragments that may be obtained from the entire polynucleotide starting material, and where the SQFs are those between the search target sites defined by steps (Mᵤ) and (Mᵤ−1) of the process-pattern used to generate each SQF-fraction.

Typically, regardless of whether DNA amplification was or was not used during the production of SQFs, the majority of the SQFs obtained in a given SQF-fraction may be uniquely distinguished (e.g., by size) by appropriate well-known polynucleotide fragment-detection techniques. Typically, for a given polynucleotide sample, the ability to distinguish the majority of SQFs (e.g., by size) obtained there from is a function of the following properties of the search target-group (Gu): the number (Mᵤ) of major search classes; the number and order of search target members in each major search class; and the mean fragment length associated with each search target, including the partition search target, in (Gu) for the given polynucleotide sample.

Pertinent references for the well-known laboratory procedures that are used in the laboratory embodiments of the present invention and that may also be noted immediately below and elsewhere include: Sambrook, J.; Russell, D.: Molecular Cloning: A Laboratory Manual, 3^{rd} ed.; Cold Spring Harbor Press, Cold Spring Harbor, 2000; and Birren, B.; Green, E. D.; Klapholz, S.; Myers, R. M.; Roskams, J.: Analyzing DNA (Genome Analysis: A Laboratory Manual Series, Vol. 1); Cold Spring Harbor Press, Cold Spring Harbor, 1997.

The annealing of two appropriate oligonucleotides to form a “ligatable”double-stranded oligonucleotide adapter of interest is well-known in the art. Also well-known in the art is the use of an appropriate DNA ligase, such as T4 DNA ligase, to effect the ligation of an appropriate, “ligatable”, double-stranded oligonucleotide adapter to DNA fragments, including those fragments immobilized on solid supports, that bear the appropriate caudemers. Also well-known in the art are several DNA amplification methods that may be suitable for use as indicated above. T4 DNA ligase and DNA amplification reagents are commercially available from Amersham Pharmacia Biotech Inc. USA (Piscataway, N.J., USA), among other suppliers. Oligonucleotides of interest, including end-labeled oligonucleotides of interest, may be obtained as highly purified products from many commercial sources.

Conditions for the elution of unbound polynucleotide strands of interest from DNA fragments immobilized by one strand of one of their termini are also well-known in the art. Finally, buffer solutions suitable for washing the immobilized DNA fragments on the solid support are well-known in the art. Typically, washing is effected using any appropriate solution or buffer that will not interfere with the binding of the DNA fragments to the solid support, nor interfere with subsequent reactions using the fragments, e.g., cleavage, TIEN derivatization, ligation, strand elution, or DNA amplification reactions. Typically, a wash or washing step may actually include several distinct cycles of wash buffer addition, brief or extended incubation, and wash buffer removal to effect the complete removal of unwanted material.

D) Variable-Scale Processing of a Polynucleotide Sample to Obtain Physical Structured Query Fragments

In the following description of a preferred laboratory embodiment of the present invention, the basic processing steps (e.g., termini-specific immobilization, washing, generation and isolation of ranked sibling progeny fractions of polynucleotide fragments, stoppage of cleavage reactions, purification or buffer exchange, re-immobilization, eventual use of double-stranded adapters and DNA amplification, etc.) are essentially as described for the “comprehensive-scale” processing of a polynucleotide sample to obtain physical structured query fragments. However, these steps are organized somewhat differently, and the general case of using any search target-group (Gu) of valid structure is described. This description of “variable-scale” processing also recognizes that at certain junctures one may elect to proceed along only certain execution paths because only certain process-patterns are of interest. However, if all possible execution paths are pursued using the search target group (Gu) and the variable-scale processing description provided below, the end result in terms of the process-patterns and SQFs of interest obtained would be exactly the same as would be obtained using “comprehensive-scale” processing of the same sample, where the “comprehensive-scale” processing was amended as appropriate for the specific structure of the search target group (Gu) (i.e., the number of major search classes and the number of search targets per major class). The steps of a variable-scale search strategy typically include those discussed in a generalized fashion in the following paragraphs.

Steps (1) through (3) are essentially as described for the “comprehensive-scale” processing described above in the previous section.

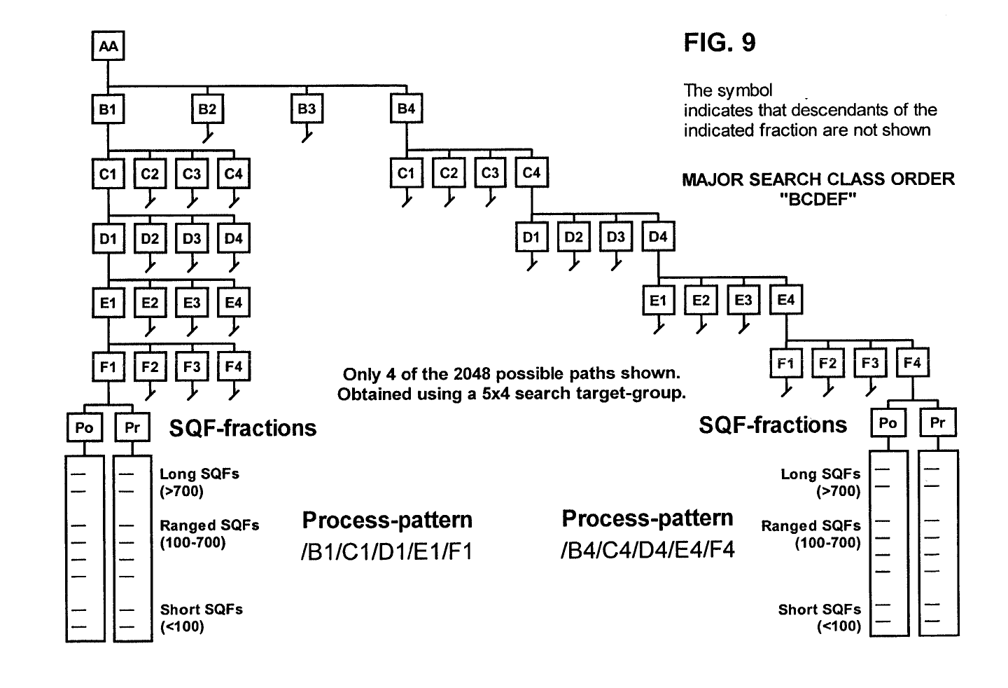

4) The resulting solution of derivatized (i.e., specifically activated) partition fragments, which typically includes symmetrically derivatized (TIEN-Qa-DNA-Qa-TIEN) and asymmetrically derivatized (TIEN-Qa-DNA-dideoxynucleotide) partition fragments, is obtained in a buffer that is suitable for activated-terminus-specific immobilization of the partition fragments to an appropriately derivatized solid support. The solution is then divided into equal aliquots that represent distinct “zero generation” parent fractions, typically up to a maximum of Mᵤ factorial aliquots. Each aliquot (CPAₖ, where typically k≦Mᵤ!) typically is processed, as described in the following sections, using one of the Mᵤ! permutations of the Mᵤ major classes of search targets defined in the search target-group (Gu). For an example of the variable-scale processing of a polynucleotide sample using a 5×4 search target-group and using a single major class permutation (“BCDEF”), see FIGS. 9 and 10.

5) Next, each aliquot (CPAₖ) is subjected to a hierarchically ordered, recursive, branching sequence of processing steps defined by the major search class permutation assigned to the aliquot. For the partition-fragment immobilization events, the only productively immobilized partition fragments on each of the “zero-generation” parent solid supports are symmetrically derivatized partition fragments (TIEN-Qa-DNA-Qa-TIEN), where the partition fragments may be immobilized, with equal probability, at one of either of their ends, and thus with either polarity. All subsequent TIEN-mediated immobilization reactions are by definition asymmetric.

For the purposes of defining the recursive sequence of steps, the initial current major search class index (i) may be initialized to zero at the very start of the following sequence of steps. For each major search class index (i), Jmaxᵢ is the number of sequence-specific double-stranded-cleavage effectors, each of whose recognition sequence is a major search target member, in the major search class (Cᵢ). The upper bound of the index value i=1, 2, . . . , Mᵤ is the number of major search classes (Mᵤ) defined by Gu.

The hierarchically ordered, recursive, branching sequence of processing steps typically is comprised of;

i) obtaining the derivatized DNA fragments in a solution suitable for activated-terminus-specific immobilization of the fragments to an appropriately derivatized solid support, and the subsequent terminal immobilization of the fragments using a physically isolated microwell or the equivalent whose surface represents the appropriately derivatized solid support. This step typically establishes the current parent fraction.

ii) blocking of unreacted binding sites (e.g., TIEN-specific binding sites) on the derivatized solid support;

iii) washing of the blocked solid support to remove any unimmobilized fragments, using any appropriate solution or buffer that will not interfere with the binding of the DNA fragments to the solid support nor interfere with any subsequent reaction involving the DNA fragments;

iv) Derivatization, typically of the 3′-hydroxyl group, of the currently immobilized DNA fragments' distal termini (i.e., termini generated by the previous sequence-specific, double-stranded cleavage step) as follows: If (i)=0 or (Mᵤ), no derivatization is required; If 0<(i)<(Mᵤ−2), derivatization is effected using a TIE-nucleoside triphosphate and TdT as described earlier; If (i)=(Mᵤ−2), derivatization may, in some laboratory embodiments, be effected using a TIE-nucleoside triphosphate and TdT as described earlier, and no derivatization is required thereafter. However, in some laboratory embodiments, when (i)=(Mᵤ−2) or (i)=(Mᵤ−1), derivatization in each case is effected by the ligation, using an appropriate DNA ligase, of a double-stranded, primer-binding-site-containing oligonucleotide adapter to the distal end of the currently immobilized DNA fragments, essentially as described for the “comprehensive-scale” processing described above in the previous section.

v) post-derivatization washing of the solid support using any appropriate solution or buffer that will not interfere with the binding of the DNA fragments to the solid support nor interfere with any subsequent reaction involving the DNA fragments;

vi) If (i)=(Mᵤ−1), in those laboratory embodiments where DNA amplification is used, the unbound strands are eluted, divided into two equal aliquots, amplified using the appropriately labeled, generic primers (Pₒ) and (Pᵣ), re-immobilized onto appropriately derivatized distinct solid supports and then washed, essentially as described for the “comprehensive-scale”processing described above in the previous section.

vii) if (i)<Mᵤ then increment (i) by one to specify the current major search class (Cᵢ); otherwise the processing steps of the current branch of the recursive procedure are complete;

viii) initialization of the value of the class member index (jᵢ)for the current class to zero;

ix) if (jᵢ)<Jmaxᵢ for the current major class, incrementing jᵢ by one to specify the current major search target Qm_{i,j} for the following steps, otherwise all of the ranked progeny fractions have been generated from the current parent fraction using the current major search class (Cᵢ);

x) complete cleavage of the terminally immobilized fragments using the sequence-specific, double-stranded cleavage effector whose recognition sequence is the current major search target Qm_{i,j}, followed by stopping of the reaction, and the removal and storage of the ranked progeny fraction containing the completed digest solution;

xi) If i<Mᵤ, calling of the recursive procedure, where a next-generation parent fraction is established at Step (i) using the jᵢ—ranked sibling progeny fraction of liberated fragments obtained in Step (x) using the current-generation parent fraction. However, if i=Mᵤ, the jᵢ—ranked sibling progeny fraction or a portion thereof is subjected to physical analysis to determine the number of, size of, and relative signal-strength associated with some or all of the structured query fragments (SQFs) found therein, as described in the section “Physical Analysis of SQFs” herein below;

xii) post-cleavage washing of the solid support using any appropriate solution or buffer that will not interfere with the binding of the DNA fragments to the solid support nor interfere with any subsequent reaction involving the DNA fragments; and

xiii) beginning the next iteration of cleavage at Step (ix) of the current branch of execution using the terminally immobilized DNA fragments that have been retained on the solid support after Step (x) of the current iteration of the current branch of execution, thereby obtaining the next-highest-ranked sibling progeny fraction using the current-generation parent fraction.

6) Through the use of the protocol described in Part (5) above one is able to generate, for each (CPAₖ) of interest, a total of {ΠJmaxᵢ} distinct process-patterns. In those laboratory embodiments where DNA amplification had been used as described above, each of these distinct process-patterns yield two “re-immobilization-orientation-specific, last-generation” ranked sibling progeny fractions (SQF-fractions). Thus, a total of {ΠJmaxᵢ*2} SQF-fractions typically may be obtained for each (CPAₖ) of interest.

In those laboratory embodiments where DNA amplification had been used as described above, and for each (CPAₖ) of interest, {ΠJmaxᵢ} SQF-fractions may be obtained using the “original” {TIEN-(Pₒ)-adapter-mediated} immobilization orientation of the “next-to-last-generation” parent fractions. Found within each of these {ΠJmaxᵢ} SQF-fractions are all of the SQFs that may be obtained using either of the possible initial search step polarities (i.e., either of the possible partition fragment initial immobilization polarities), in all of the ACP (partition) fragments that may be obtained from the entire polynucleotide starting material, and where the SQFs are those between the search target sites defined by steps (Mᵤ) and (Mᵤ−1) of the process-pattern and the re-immobilization orientation used to generate each SQF-fraction.

Similarly, in those laboratory embodiments where DNA amplification had been used as described above, and for each (CPAₖ) of interest, {ΠJmaxᵢ} SQF-fractions may be obtained using the “reverse” {TIEN-(Pᵣ)-adapter-mediated} immobilization orientation of the “next-to-last-generation” parent fractions. Found within each of these {ΠJmaxᵢ} SQF-fractions are all of the SQFs that may be obtained using either of the possible initial search step polarities (i.e., either possible partition fragment initial immobilization polarities), in all of the ACP (partition) fragments that may be obtained from the entire polynucleotide starting material, and where the SQFs are those between the search target sites defined by steps (Mᵤ) and (Mᵤ−2) of the process-pattern and the re-immobilization orientation used to generate each SQF-fraction.

In those laboratory embodiments where DNA amplification is not used, and for each (CPAₖ) of interest, there are only {ΠJmaxᵢ} “last-generation” ranked sibling progeny fractions (SQF-fractions), each of which was obtained using one of the {ΠJmaxᵢ} distinct process-patterns that are obtainable. These {ΠJmaxᵢ} SQF-fractions contain SQFs of the “original” immobilization orientation used to produce the “next-to-last-generation” parent fractions. Found within each of these {ΠJmaxᵢ} SQF-fractions are all of the SQFs that may be obtained, using either of the possible initial search step polarities (i.e., either of the possible partition fragment initial immobilization polarities), in all of the ACP (partition) fragments that may be obtained from the entire polynucleotide starting material, and where the SQFs are those between the search target sites defined by steps (Mᵤ) and (Mᵤ−1) of the process-pattern used to generate each SQF-fraction.

E) Physical Analysis of SQFs

As described above and in the preceding section, a DNA amplification procedure may be included in the laboratory embodiments of the disclosed invention. For a given SQF analysis or process-pattern(s) defined thereby, this procedure typically is used to accomplish one or more of the following objectives: to double the number of SQF-fractions obtained using each process-pattern; to obtain adequate amounts of detection-reagent-labeled SQFs for subsequent analytical purposes; or to obtain adequate amounts of unlabeled SQFs for subsequent analytical or preparative purposes.

Many polynucleotide amplification procedures are known in the art and can be used with the present invention, including the use of the polymerase chain reaction (PCR) with “generic” primers. In this discussion and hereafter a “generic” PCR primer is one that is solely and specifically intended to bind to and permit primer extension from a synthetic primer-binding (annealing) site that had been artificially introduced at the end of a DNA fragment by the ligation thereto of a double-stranded oligonucleotide adapter that contains the primer-binding site in the proper orientation. Thus, a “generic” PCR primer is designed so that it typically will not anneal to, and therefore will not permit primer extension from, any naturally occurring sequence in the physical sample(s) of polynucleotides under study.

The polymerase chain reaction can be conveniently introduced and used in the laboratory preferred embodiment of the disclosed invention. When PCR is used in this embodiment, it is implemented using two different “generic” primers whose synthetic annealing sites are introduced artificially by the step-wise ligation of double-stranded oligonucleotide adapters of known sequence on either end of the fragments to be amplified. These steps are carried out in a manner that does not affect the process-patterns required to produce the desired end products (structured query fragments) of the laboratory preferred embodiments of the disclosed invention. Only two different types of PCR primers are required to amplify extremely large numbers of SQFs. These SQFs have partially characterized sequence properties that typically allow them to be mapped automatically to a reference dataset using the computational preferred embodiment of the disclosed invention. Furthermore, once a generic PCR primer has been found to perform satisfactorily for one or more sources of polynucleotides, it may be used in all laboratory SQF analyses that use samples from these sources of polynucleotides, regardless of the specific search target-group used for these analyses. The unique manner and scale with which PCR can be used during laboratory SQF analyses as described above addresses the inherent scalability problems associated with attempts to implement conventional, locus-specific PCR or related locus-specific amplification techniques on a genomic scale.

Physical techniques are used to analyze SQF-fractions and the SQFs therein. The method used (if any) to label the fragments during the above-described procedures may limit those fragment analysis techniques that are appropriate. These fragment analysis techniques can be selected based on their ability to resolve, preferably at nucleotide resolution, the SQFs in each SQF fraction that fall within a suitable or desired size range, and that typically are referred to as “ranged” SQFs. When the fragment analysis technique is denaturing capillary electrophoresis using fluorescence-based detection, a suitable or desired size range for the resolution of the labeled strands of the ranged SQFs present in an SQF-fraction may be between 100 to 700 nucleotides. When the fragment analysis technique is HPLC, mass spectrometry (MS), or combined HPLC-MS, a suitable or desired size range for the resolution of double-stranded ranged SQFs present in an SQF-fraction may be between 50 to 550 nucleotide pairs. For any of these or other fragment analysis techniques, the actual lower and upper fragment analysis limits that may be used to define “ranged” SQFs may vary considerably from those indicated, depending on the instrumentation and analysis conditions used.

Although typically physical analysis techniques may be most conveniently applied to individual SQF fractions or known combinations thereof, in some embodiments one may wish to obtain a more detailed characterization of one or more individual SQF fragments within an SQF fraction of interest. Thus, as a non-limiting example, one may use preparative capillary electrophoresis to isolate individual fragments that may then be obtained in an appropriate buffer and used as a template for conventional DNA sequencing reactions, typically dideoxy sequencing reactions. Alternatively, the DNA sequence of each fragment in individual SQF fractions or known combinations thereof may be determined in parallel using newer DNA sequencing methodologies, such as “sequencing by hybridization”. Conventional DNA sequencing reactions are well-known in the art (see e.g., Sambrook, J.; Russell, D.: Molecular Cloning: A Laboratory Manual, 3^{rd} ed.; Cold Spring Harbor Press, Cold Spring Harbor, 2000; and Birren, B.; Green, E. D.; Klapholz, S.; Myers, R. M.; Roskams, J.: Analyzing DNA (Genome Analysis: A Laboratory Manual Series, Vol. 1); Cold Spring Harbor Press, Cold Spring Harbor, 1997) and “sequencing by hybridization” (U.S. Pat. No. 5,202,231).

The SQF fragment analysis data obtained in the laboratory embodiments of the present invention may be recorded in a relational database application, together with other known information, in a single table or two or more related tables. For each laboratory SQF detected, i.e., each distinct fragment resolved and detected among the limited number of SQFs present in an SQF-fraction, a record may be created in the table (or appropriately joined tables), where each such record may include non-nullable data fields or data field combinations whose data values may serve as unambiguous identifiers of one or preferably all of the following: the analytical method used; the instrument used; the DNA sample used; the process-pattern; for certain embodiments, the re-immobilization orientation used {TIEN-(Pₒ) or TIEN-(Pᵣ)} prior to the use of the final major search class (Mᵤ); the estimated size of the SQF, where the estimated size of the SQF may be uncorrected or corrected for the addition of a “generic” labeling primer (as described earlier); size standards used; the relative strength of the signal attributed to the detection of the SQF; the adapters, primers, and detection labels (if any) used in the generation of the SQF; an identifier of the particular experiment performed (e.g., date of experiment); and other data that may be relevant to a complete description of each SQF.

Some of the computationally predicted SQF entities are designated as possessing “site conflicts” due to violation of the effective functional boundary requirements of the sequence-specific, double-stranded cleavage effectors that would be required to produce the corresponding laboratory SQF entities. As described above in one of the computational preferred embodiments, it is possible to calculate the effective functional boundary required by and resulting from a cleavage event at any search target (Q) site, using the values of the cut-offsets CO_{ds} and CO_{rc}, and the length of Q, for any search target (Q) in (Gu).

The information acquired and stored in the laboratory SQF database may be used to compare the physical SQF entities with the computationally predicted SQF entities obtained using the same search target-group (Gu) as was used in the laboratory SQF analysis, and a sequence dataset (D) that contains the sequences of all of the ACP fragments expected to be present in the physical sample of DNA used for the laboratory SQF analysis. These comparisons typically are most useful when computationally predicted SQF entities that lack site-conflicts (or whose predicted site conflicts do not prevent the generation of the corresponding laboratory SQF entities) are compared with the corresponding laboratory SQF entities that share the same process-pattern definition and size. The estimated size of each laboratory SQF may, where necessary, be corrected for the addition of the generic labeling primer (Pₒ) or (Pᵣ) that was required to generate the laboratory SQF as described earlier.

III. Specific Applications Using the Current Invention

A) DNA Methylation Analysis

A preferred embodiment of the present invention is a laboratory SQF analysis procedure that can be used to identify naturally-occurring methylation sites in a sample of DNA. Generally, for these embodiments, a “comprehensive” or “variable” scale laboratory SQF analysis is carried out as previously described. However, the search target-group that is used contains one or more search targets that is the recognition sequence for a methylation-sensitive, sequence-specific double-stranded cleavage effector. Typically, each of the search targets is a CG-dinucleotide-containing recognition sequence for a cytosine-methylation-sensitive Type 11 restriction enzyme that cannot cleave its recognition sequence in DNA if the site contains an internal methylated cytosine residue, typically the cytosine residue in the CG-dinucleotide of its recognition sequence.

These SQF analyses are typically carried out using DNA samples from mammalian species, where it is well-known that methylation of cytosine residues at the 5-position of the pyrimidine ring is a naturally occurring DNA modification at certain sites in DNA. Cytosine methylation typically occurs at some but not all CG dinucleotide sites in mammalian DNA, and may have significant effects on a variety of genetic and epigenetic phenomena that have important biological consequences. Thus, determining the location of these cytosine methylation sites in mammalian DNA is of considerable importance, but existing methods typically are difficult if not impossible to carry out on a genome-wide scale.

Typically, in these laboratory embodiments each of the major search targets in the search target-group is a recognition sequence for a methylation-insensitive, sequence-specific double-stranded cleavage effector, whereas the partition search target (Qa), is a recognition sequence for a methylation-sensitive, sequence-specific double-stranded cleavage effector. Thus, as a non-limiting example, the restriction enzyme Hpa II may be used as the sequence-specific double-stranded cleavage effector whose recognition sequences is the partition search target (sQa) in the search target-group (sGu). The cleavage effector Hpa II cleaves its recognition sequence [CCGG] but cannot cleave [C^{m5}CGG]. Typically, physical SQFs are identified that cannot be detected using the sample of DNA and (sGu), typically in all of the SQF-fractions of interest, but that are detectable in the corresponding computational process-patterns using the same search target-group (sGu). Where possible, the locations of methylated cytosine residues [C^{m5}CGG] at the termini of SQF-yielding ACP fragments in the DNA sample under study are determined or inferred based on the known locations of the appropriate computationally predicted SQFs whose physical counterparts could not be obtained using sGu. The appropriate computationally predicted SQFs are obtained using a primary dataset that contains the known DNA sequences for the physical sample of DNA under study.

In some embodiments two different search target groups may be used, each for a separate SQF analysis using aliquots of the same DNA sample, where the two search target-groups are identical except for the methylation sensitivity of the partition search target (Qa). As before, each of the major search targets in both search target-groups is a recognition sequence for a methylation-insensitive, sequence-specific double-stranded cleavage effector. Thus, as a non-limiting example, the restriction enzymes Hpa II and Msp I may be used as the sequence-specific double-stranded cleavage effectors whose recognition sequences are the partition search targets sQa and nQa, respectively, for two otherwise identical search target-groups sGu and nGu, respectively.

The cleavage effectors Hpa II and Msp I have identical recognition sequences [CCGG] and cut-offsets. However, Hpa II cannot cleave [C^{m5}CGG] sites whereas Msp I can. Thus, the laboratory SQFs obtained using (nGu) and (sGu) are typically compared in order to identify laboratory SQFs that can be detected using one aliquot of DNA and the search target-group (nGu), typically in all of the SQF-fractions of interest, but that are not detectable in the same fraction obtained using the other aliquot of DNA and the search target-group (sGu). Where possible, the locations of methylated cytosine residues [C^{m5}CGG] at the termini of SQF-yielding ACP fragments in the DNA sample under study are determined or inferred based on the known locations of the appropriate computationally predicted SQFs whose physical counterparts were obtained using (nGu) but not with (sGu). The appropriate computationally predicted SQFs are obtained using a primary dataset that contains the known DNA sequences for the physical sample of DNA under study.

There are other possible embodiments of laboratory SQF analyses that may be used to study DNA. These include, but are not limited to, embodiments where the search target-group includes one or more major search targets that is the recognition sequence for a methylation-sensitive, sequence-specific double-stranded cleavage effector. An important proviso here is that when DNA amplification is used as described above during the generation of SQFs, then methylation sites at the recognition sequences for the search target members of the last major search class used (i.e., after DNA amplification) cannot be examined using SQFs derived from these process-patterns.

B. Use of SQFs in Hybridization reactions.

Physical SQFs generated using the laboratory procedures described above can be used, typically as a degenerate pool (i.e., a SQF-fraction) of partially characterized polynucleotide hybridization probes, in virtually any current laboratory methodology employing polynucleotide hybridization probes. Polynucleotide hybridization reaction conditions are well-known in the art, and are reviewed in Cantor, C. R.; Smith, C. L. Genomics: The Science and Technology Behind the Human Genome Project; Wiley: New York, 1999; Sambrook, J.; Russell, D.: Molecular Cloning: A Laboratory Manual, 3^{rd} ed.; Cold Spring Harbor Press, Cold Spring Harbor, 2000; and Birren, B.; Green, E. D.; Klapholz, S.; Myers, R. M.; Roskams, J.: Analyzing DNA (Genome Analysis: A Laboratory Manual Series, Vol. 1); Cold Spring Harbor Press, Cold Spring Harbor, 1997.

Typically, one of the SQF-fractions is used as a degenerate pool of partially characterized polynucleotide probes. In general, a positive hybridization signal obtained using the appropriate stringency indicates that at least one polynucleotide sequence, or a region therein, in the test sample is capable of complementary base-pairing with at least one SQF sequence, or a region therein, in the SQF-fraction used in the hybridization reaction. Ambiguity as to which SQF sequence(s) had actually been involved in complementary base-pairing with one or more polynucleotide sequence(s) in the test sample may be reduced if not eliminated altogether through parallel hybridization reactions involving the same test sample with as many different SQF-fractions of interest as is deemed necessary.

Hybridization reactions using SQF-fractions may be carried out on spatially addressable microarrays (hereafter referred to as “microarrays”). Virtually any microarray technology can be used with the-present invention. For example, SQF-fractions can be immobilized on microarrays similar to those described in U.S. Pat. No. 5,807,522. Microarrays containing SQF-fractions can be used for a variety of useful preparative and analytical procedures that previously relied on the use of polynucleotide hybridization probes obtained directly by recombinant DNA cloning, or as synthetic oligonucleotides whose sequence was determined from a polynucleotide fragment obtained by recombinant DNA cloning. Some important examples of procedures involving the use of SQF-fractions as hybridization probes include the identification and mapping of RNA transcripts, gene discovery, and quantitative analyses of gene expression.

In one preferred embodiment, SQF-fractions obtained using the present invention may enable the detect or isolation (or both) of transcribed polyribonucleotide sequences that are difficult if not impossible to detect or isolate by existing methods. Examples of such transcripts include low copy number RNA transcripts, or RNA transcripts that are directly (as polyribonucleotides) or indirectly (e.g., because of a protein product or products derived there from) deleterious to any host species (e.g., E. coli) or strain that one may attempt to use during molecular cloning of a cDNA representing the RNA transcripts.

As a non-limiting example, SQF-fractions may be used as a hybridization substrate to be spotted individually (using one fraction per spatially addressable spot) on one or more microarrays. Each spatially addressable spot on the DNA microarray(s) may contain an SQF-fraction of interest and of known process-pattern. Any signal generated by the hybridization, using appropriate stringency, of a labeled test polynucleotide sample to a given spot on the microarray can be assumed to have arisen due to the hybridization of one or more specific polynucleotides in the labeled sample to one or more SQFs in the SQF-fraction represented by the spot, and thus involve complementary base-pairing to one or more of only a limited number of possible locations in the DNA sample used to generate the SQFs. These locations may be determined in a sequence dataset (D) that contains the sequences of all of the ACP fragments expected to be present in the physical sample of DNA used to generate the laboratory SQFs, using the computationally generated SQFs of the same process-pattern definition as that which characterizes the laboratory SQF-fraction used for the given spot. More than one SQF-fraction may be included in a spatially addressable spot. Furthermore, one or more of the precursor DNA fractions (i.e., fragments liberated from a defined immobilized substrate with current major search class Cᵢ where i<Mᵤ) used in the generation of laboratory SQFs may be used in a spot, where each such fraction is spotted individually (using one such fraction per spatially addressable spot), or where certain precursor DNA fractions may be pooled.

In another non-limiting example of hybridization reactions using physical SQFs, one or more SQF-fractions of interest may be labeled and used as a hybridization probe for screening a library of molecular clones. In other embodiments, the physical material to be labeled and used as a hybridization probe may be one or more of the precursor DNA fractions of interest (i.e., fragments liberated from a defined immobilized substrate with current major search class Cᵢ where i<Mᵤ) used in the generation of laboratory SQFs.

C. Cloning of SQFs.

In another embodiment of the present invention, one or more SQF-fractions of interest, or one or more DNA fragment precursor fractions (described above) of interest, may be used to construct one or more libraries of molecular clones of the SQFs. Molecular cloning methods, including methods for constructing libraries of molecular clones from polynucleotide samples, are well-known in the art and are described in: Sambrook, J.; Russell, D.: Molecular Cloning: A Laboratory Manual, 3^{rd} ed.; Cold Spring Harbor Press, Cold Spring Harbor, 2000.

D.) Structure-Based Annotation of Primary Datasets and Comparative Analyses using Process-Patterns and SQFs

SQF analysis has an essentially unlimited potential to generate computational annotations of one or more sets of strings of interest, where each such set of strings is typically a large to extremely large primary dataset of biopolymer sequences. These computational annotations are structure-based and are comprised of the process-pattern entities, and the SQFs found therein, that are obtained throughout the dataset or datasets of interest using the search target-groups of interest. For any given dataset of interest, or for comparisons of different datasets of interest, multiple search target-groups may be defined and used to obtain structure-based annotations that taken together may attain any desired density or complexity. The power and flexibility of SQF analysis in this regard is only limited by the ability of investigators in the research community to design search target-groups that generate process-pattern entities or their SQF derivatives that reveal the structure-based similarities or differences of interest in the dataset or datasets.

Thus, a single process-pattern, or two or more process-patterns taken as a group, or a single SQF, or two or more SQFs taken as a group, that may be obtained from one or more ACP fragments in each string (Xᵢ) in a set (X) of one or more unique strings in the relational database application, may be used to define membership in the set (X), and thus must be present in each (Xᵢ) or one or more substrings of each (Xᵢ) (and the entity or entities that each Xᵢ or one or more substrings of each Xᵢ may represent) and thereby distinguish the members of the set (X) from other strings (zⱼ) (and the entities that the other strings zⱼ may represent). None of the ACP fragments obtainable from (zⱼ) yield the single process-pattern, or two or more process-patterns taken as a group, or single SQF, or two or more SQFs taken as a group, that define membership in (X). The presence of the process-pattern, group of process-patterns, SQF, or group of SQFs thereby establish a classification system for some or all of the strings or substrings therein (and the entities that the strings or substrings therein may represent) analyzed by the relational database application. The classification system may reflect and reveal underlying structural, functional, phylogenetic, or other useful properties that are common or related among the entities that may be represented by the strings or substrings analyzed by the relational database application of the present invention.

The relational database application developed for a computational preferred embodiment of the present invention even allows an entire linear sequence (e.g., a chromosome) to be treated as one large partition fragment. Thus, using this SQF analysis option, or using the conventional search target-group structure, appropriately designed search target-groups may be used in computational analyses to obtain process-patterns whose search target sites span very large regions of a chromosome, and allow for comparative analyses at, say, the level of whole chromosomes, or at the level of very large subregions therein.

The ability to use the results of computational SQF analyses for the structure-based annotation and classification of polynucleotide sequences has many other important applications. In some cases, a single process-pattern, or two or more process-patterns taken as a group, or a single SQF, or two or more SQFs taken as a group, may act as a unique or diagnostic molecular signature or fingerprint for the identification of a genome, or a subregion thereof that may be of interest. In a similar manner, a single process-pattern, or two or more process-patterns taken as a group, or a single SQF, or two or more SQFs taken as a group, may act as a unique or diagnostic molecular signature or fingerprint for the identification of a specific individual's genome, or a subregion thereof that may be of interest, where the individual may need to be identified for some reason, non-limiting examples of which may include a medical diagnosis, tissue or organ transplant, or forensic or other type of identification.

Other important applications include the comparison of laboratory SQF data and computational SQF data using a given sequence dataset, where the comparison may be used to help prevent, identify, or correct errors or ambiguities in the sequence dataset. These sequencing errors or ambiguities may have arisen during the assembly of individual sequencing fragments into larger contiguous sequences (contigs), or they may have arisen during molecular cloning procedures required to produce the DNA sequencing templates used to generate the sequence data in question. Thus, if a given computational process-pattern is known to span the overlap region of two individual sequences that were subsequently joined to form one contiguous sequence, and the corresponding physical SQFs may be obtained using the process-pattern and an appropriate DNA sample or samples, then the agreement between the computationally predicted and observed data validates the joining or assembly of the two individual sequences. Similarly, any accepted reference sequence for a given genome may be annotated wherever its computationally predicted SQFs cannot be obtained as their corresponding physical SQF counterparts using an appropriate DNA sample or samples, especially where (as is typically the case) the computationally predicted SQFs do not contain any “site-conflicts” as described above.

In certain embodiments, comparative laboratory SQF analyses may be performed using a common search target-group (Gu) and two aliquots of the same physical sample of DNA to determine the effects of a test treatment on a DNA sample. Such a test treatment may include, as non-limiting examples, exposure to radiation or a suspected or known carcinogen, mutagen, or teratogen or the like, or a mixture of two or more of the exposures. For these experiments, one DNA aliquot, referred to as the “control” aliquot, is not subjected to any further treatment. The other DNA aliquot, referred to as the “test” aliquot, is subjected to some enzymatic, chemical, or physical process or exposure of interest, or to some combination of enzymatic, chemical, or physical processes or exposures of interest. In these embodiments, the treatment is referred to as the “test” treatment. The laboratory SQFs obtained using the control aliquot and the test aliquot are compared in order to determine the effect(s), if any, that the test treatment has on the DNA sample under study, as reflected by the detection of laboratory SQFs using one aliquot of DNA that are not detectable using the other aliquot. Furthermore, where possible, the location(s) of the effect(s) in the polynucleotide may be determined based on the known locations of the computationally predicted SQFs obtained using a primary dataset that contains known DNA sequences for the physical sample of DNA under study.

In certain embodiments, comparative SQF analyses are performed using a common search target-group (Gu) and two physical samples of DNA of comparable purity and physical integrity, that are isolated from biological samples that are derived from the same individual (or genetically identical individuals), to determine the effects of a test treatment on the biological samples. For example, the biological samples may be tissue samples, or cell-cultures, or the like. For these embodiments, one DNA sample, referred to as the “control” sample, is derived from a biological sample that has not experienced or been subjected to the “test” treatment. The other DNA sample, referred to as the “test” sample, is derived from a biological sample that has experienced or been subjected to some biological, chemical, or physical process or exposure of interest, or to some combination of biological, chemical, or physical processes or exposures of interest. The laboratory SQFs obtained using the control sample and the test sample are compared in order to determine the effect(s), if any, that the test treatment has on the DNA sample under study, as reflected by the detection of laboratory SQFs using one sample of DNA that are not detectable using the other sample. Furthermore, where possible, the location(s) of the effect(s) in the polynucleotide may be determined based on the known locations of the computationally predicted SQFs obtained using a primary dataset that contains the known DNA sequences for the physical sample of DNA under study.

In certain embodiments the “control” sample is derived from a biological sample that does not exhibit a trait (desirable or undesirable phenotype) under study, whereas the other DNA sample, referred to as the “trait” sample, is isolated from a tissue or tissues, or cell-culture, or the like, that does exhibit the trait under study but that is from the same species as the “control” sample, and to the extent possible, is otherwise as genetically similar to the “control” sample as possible.

In these and similar embodiments (e.g., using a “control” sample and a “trait” sample), the laboratory SQFs obtained using the control sample and the trait sample are compared in order to identify laboratory SQFs that can be detected using one sample of DNA, but that are not detectable using the other sample. Furthermore, where possible, the location(s) of the differences(s) are determined based on the known locations of the computationally predicted SQFs obtained using a primary dataset that contains the known DNA sequences for the physical samples of DNA under study. These embodiment may be useful for identifying DNA regions (or their possible gene expression products) that are involved in the trait under study, or determining DNA regions whose physical integrity may be affected by the trait under study, or both.

Thus, in other embodiments, comparative SQF analyses may be performed using a common search target-group (Gu) and physical samples of DNA obtained from two distinct populations of unrelated individuals. For these embodiments, each individual in one population (“control”) does not exhibit the trait (desirable or undesirable phenotype) under study, whereas each individual in the other population (“trait”) does exhibit the trait under study. In yet other embodiments, comparative SQF analyses are performed using a common search target-group (Gu) and physical samples of DNA obtained from two distinct populations of related individuals, where each individual in one population (“control”) does not exhibit the trait (desirable or undesirable phenotype) under study, whereas each individual in the other population (“trait”) does exhibit the trait under study.

E.) Comparative SQF Analysis for the Detection of Identity-By-Descent

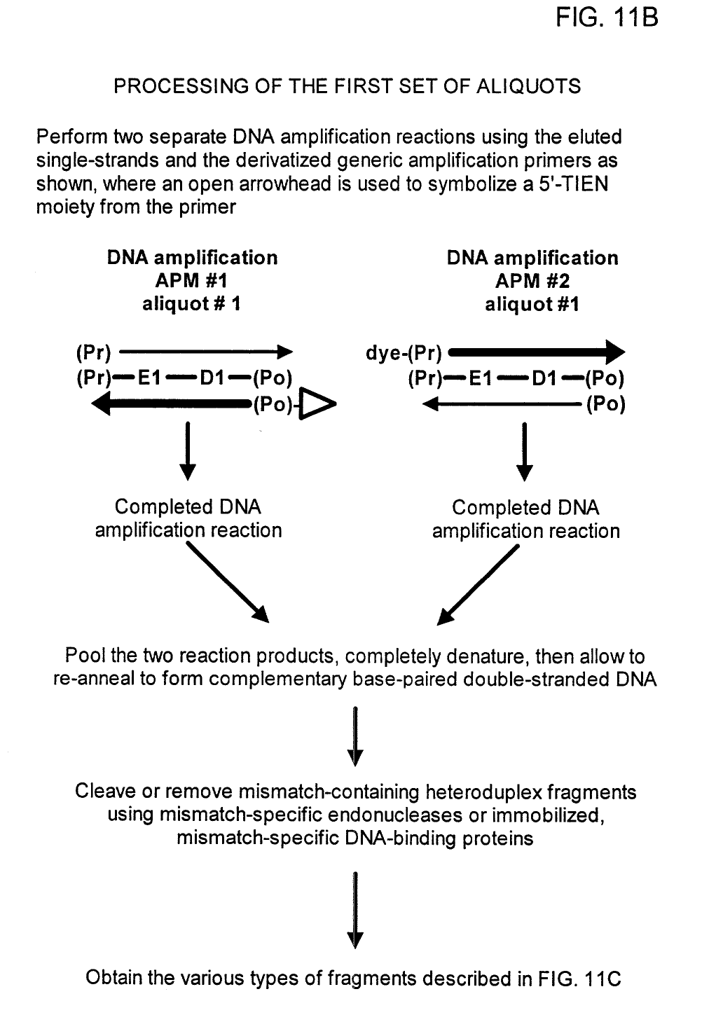

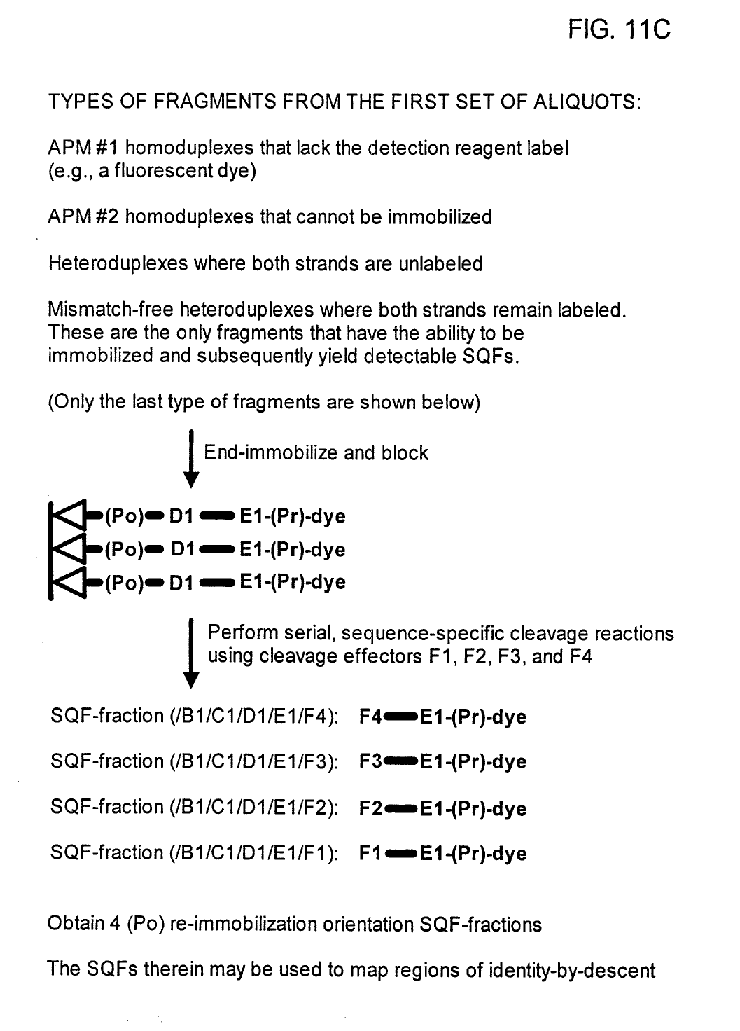

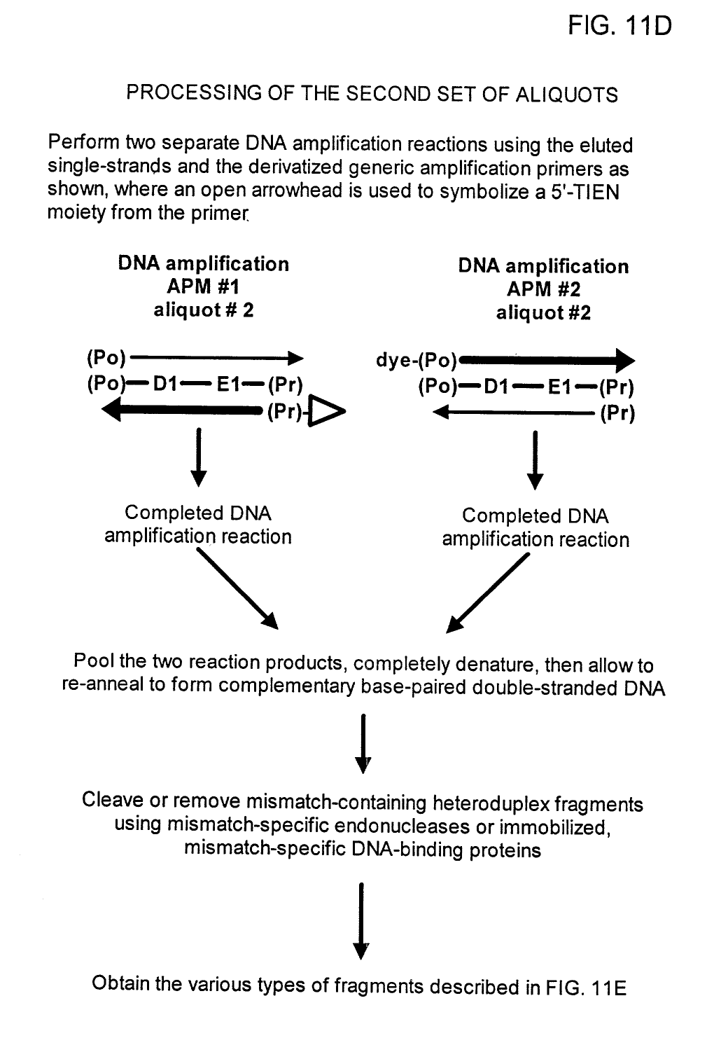

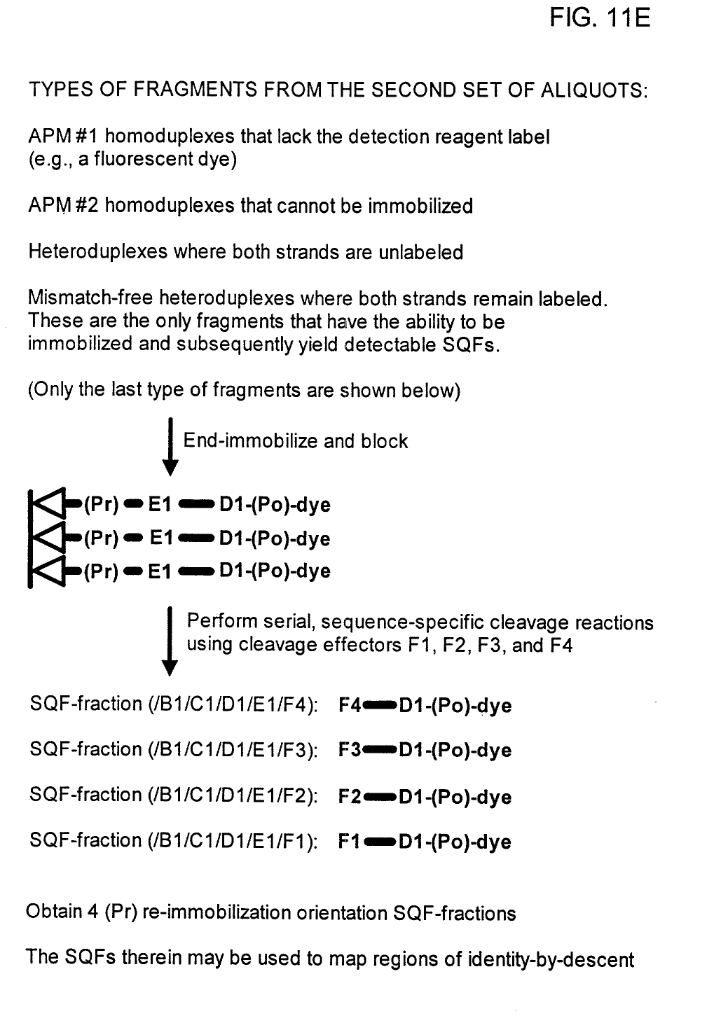

The comparative SQF analysis methods of the present invention may be used in affected pedigree member (APM) studies for identifying DNA regions (or their possible gene expression products) that are identical-by-descent, and that may be involved in the trait under study. For these studies, comparative SQF analyses are performed using a common search target-group (Gu) and physical samples of DNA obtained from one or more pairs of individuals that all exhibit the trait (desirable or undesirable phenotype) under study (FIG. 11A). Each pair (APM pair) is comprised of two “affected pedigree members” (APM) designated as (APM₁) and (APM₂), where the (APM) are two relatives. For each APM pair, laboratory SQFs are generated in parallel using DNA samples obtained from each APM as described in the laboratory methods described above. However, the DNA amplification steps of these two distinct laboratory procedures are typically modified in parallel as described below (FIG. 11B). These parallel modifications affect pairs of “next-to-last generation” fractions obtained as described earlier, where the pairs of fractions are each comprised of one fraction obtained using DNA from (APM₁) and another fraction obtained using DNA from (APM₂), and where the two fractions in a pair have the same process-pattern definition up to and including the “next-to-last generation” processing step as described earlier.

a) For the “next-to-last generation” fraction from (APM₁), carrying out PCR amplification (or the equivalent) of the first of the two aliquots of eluted DNA, using a (Pₒ) primer bearing a (TIEN) residue or the equivalent at the 5′-position, and an unlabeled (Pᵣ) primer (FIG. 11B). In a separate reaction, carrying out PCR amplification (or the equivalent) of the second of the two aliquots of eluted (APM₁) DNA, using a (Pᵣ) primer bearing a (TIEN) residue or the equivalent at the 5′-position, and an unlabeled (Pₒ) primer (FIG. 11D); and

b) For the “next-to-last generation” fraction from (APM₂), carrying out PCR amplification (or the equivalent) of the first of the two aliquots of eluted DNA, using an unlabeled (Pₒ) primer, and a (Pᵣ) primer bearing a detection-reagent label (such as, but not limited to, one of the commonly used fluorescent sequencing labels) at the 5′-position (FIG. 11B). In a separate reaction, PCR amplification (or the equivalent) of the second of the two aliquots of eluted (APM₂) DNA, is carried out using an unlabeled (Pᵣ) primer, and a (Pₒ) primer bearing a detection-reagent label (such as, but not limited to, one of the commonly used fluorescent sequencing labels) at the 5′-position (FIG. 11D);and

c) combining the DNA amplification products obtained using the “first aliquot” reactions described in Steps (a) and (b) above, then completely denaturing the mixture for a brief period of time using elevated temperature, and then decreasing the temperature slowly to allow the DNA strands to form complementary base-paired, double-stranded DNA molecules that are either unlabeled hetero-hybrids (with strands generated by unlabeled (Pₒ)_{stepB} and (Pᵣ)_{stepA} primers), singly labeled homo-hybrids of two types (with, in one case, strands generated by TIEN-labeled (Pₒ)_{stepA} and unlabeled (Pᵣ)_{stepA} primers; and in the other case, strands generated by unlabeled (Pₒ)_{stepB} and detection-reagent-labeled (Pᵣ)_{stepB} primers), and most importantly, double-labeled hetero-hybrids (with strands generated by TIEN-labeled (Pₒ)_{stepA} and detection-reagent-labeled (Pᵣ)_{stepB} primers); and where;

(i) the mixture of homo-hybrid and hetero-hybrid double-stranded DNA molecules are then treated with a mismatch-specific cleavage effector which are known in the art (for example, see U.S. Pat. No. 5,824,471), where the treatment creates a cleavage point in mismatch-containing double-stranded DNA molecules, or alternatively where the treatment may involve the physical removal of mismatch-containing double-stranded DNA molecules by way of their binding to and removal by, for example, mismatch-specific DNA binding proteins, which are known in the art, immobilized on a solid support;

(ii) the resumption of the previously described method where the double-stranded DNA molecules obtained from combining the two DNA amplification reaction products as described above for a given matched pair of (APM₁) and (APM₂), “next-to-last-generation” fractions are re-immobilized on a distinct solid support using the “original” or TIEN-(Pₒ) orientation (FIG. 11C); and where the only double-stranded DNA molecules capable of immobilization and subsequent generation of a detectable SQF signal are mismatch-free, double-labeled hetero-hybrids (with strands generated by TIEN-labeled (Pₒ)_{stepA} and detection-reagent-labeled (Pᵣ)_{stepB} primers); and

d) combining the DNA amplification products obtained using the “second aliquot” reactions described in Steps (a) and (b) above, then completely denaturing the mixture for a brief period of time using elevated temperature, and then decreasing the temperature slowly to allow the DNA strands to form complementary base-paired, double-stranded DNA molecules that are either unlabeled hetero-hybrids (with strands generated by unlabeled (Pₒ)_{stepA} and (Pᵣ)_{stepB} primers), singly labeled homo-hybrids of two types (with, in one case, strands generated by unlabeled (Pₒ)_{stepA} and TIEN-labeled (Pᵣ)_{stepA} primers; and in the other case, strands generated by detection-reagent-labeled (Pₒ)_{stepB} and unlabeled (Pᵣ)_{stepB} primers), and most importantly, double-labeled hetero-hybrids (with strands generated by detection-reagent-labeled (Pₒ)_{stepB} and TIEN-labeled (Pᵣ)_{stepA} primers); and where;

(i) the mixture of homo-hybrid and hetero-hybrid double-stranded DNA molecules are then treated with a mismatch-specific cleavage effector which are known in the art (for example, see U.S. Pat. No. 5,824,471), where the treatment creates a cleavage point in mismatch-containing double-stranded DNA molecules, or alternatively where the treatment may involve the physical removal of mismatch-containing double-stranded DNA molecules by way of their binding to and removal by, for example, mismatch-specific DNA binding proteins immobilized on a solid support;

(ii) the resumption of the previously described method, where the double-stranded DNA molecules obtained from combining the two DNA amplification reaction products as described above for a given matched pair of (APM₁) and (APM₂), “next-to-last-generation” fractions are re-immobilized on a distinct solid support using the “reverse” or TIEN-(Pᵣ) orientation (FIG. 11E); and where the only double-stranded DNA molecules capable of immobilization and subsequent generation of a detectable SQF signal are mismatch-free, double-labeled hetero-hybrids (with strands generated by detection-reagent-labeled (Pₒ)_{stepB} and TIEN-labeled (Pᵣ)_{stepA} primers); and

e) determining the location(s) of the mismatch-free hetero-hybrid duplexes based on the known locations of the computationally predicted SQFs obtained using a primary dataset that contains the known DNA sequences for the physical samples of DNA under study; and where the comparative SQF analyses may be useful for identifying DNA regions (or their possible gene expression products) that are identical-by-descent, and that may be involved in the trait under study.

IV. SQF Simulation Method.

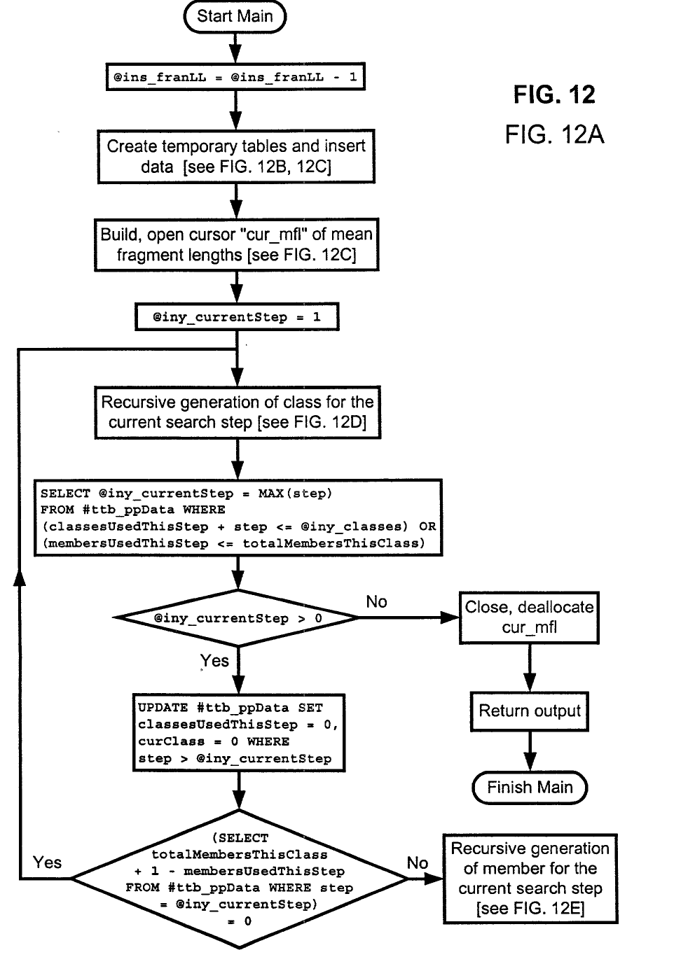

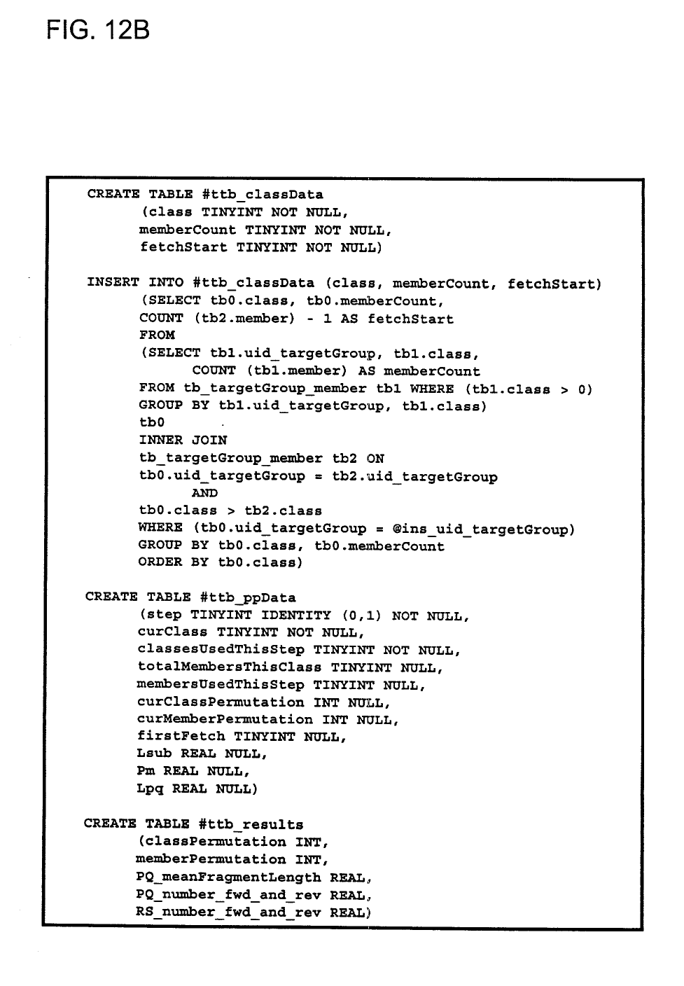

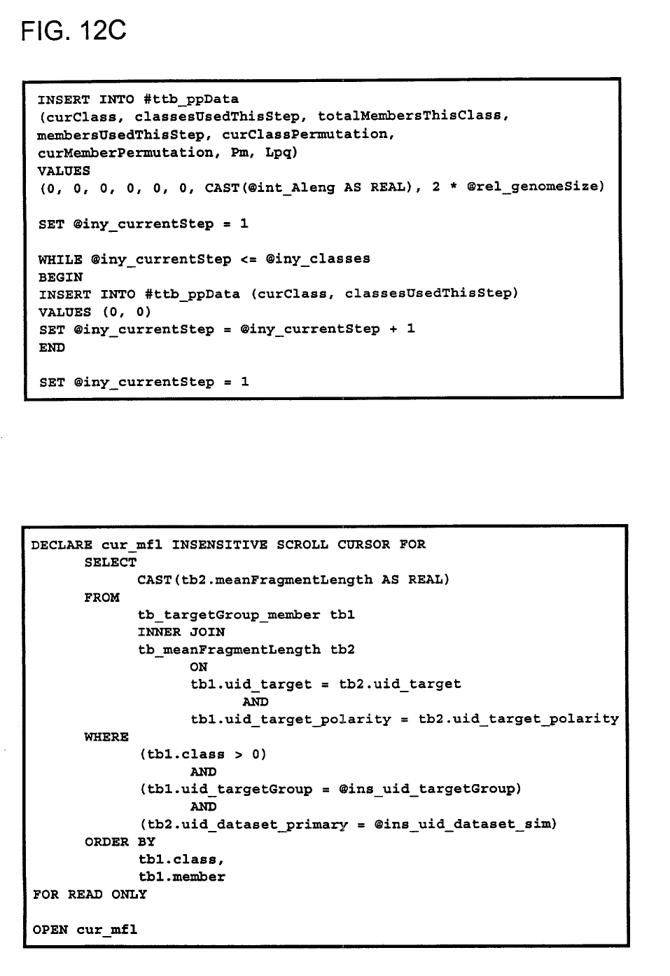

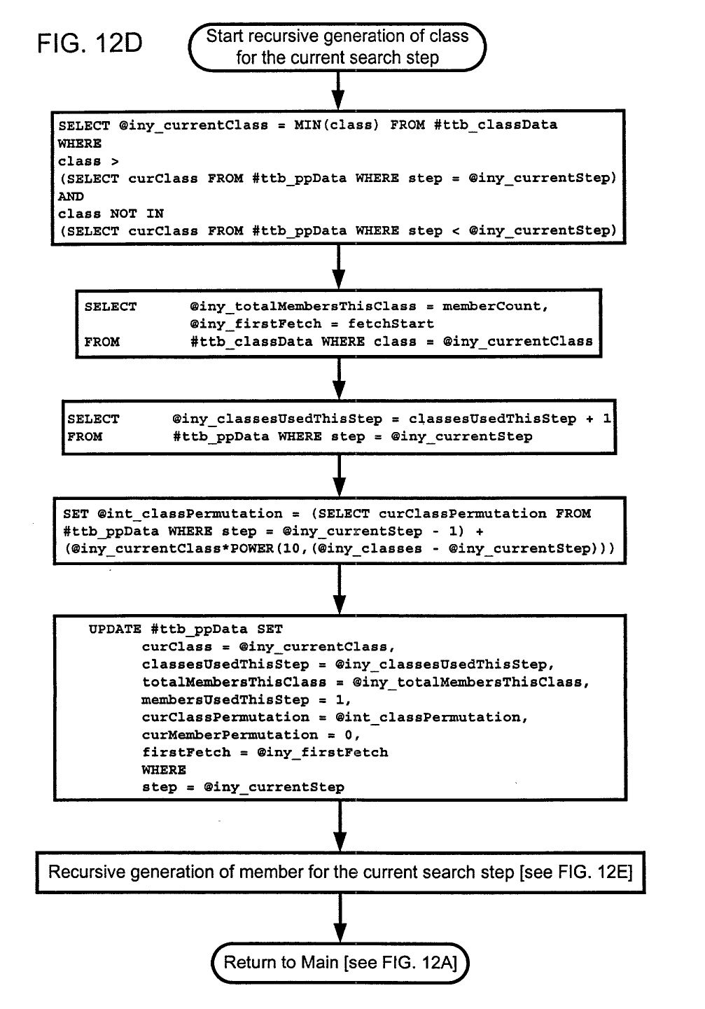

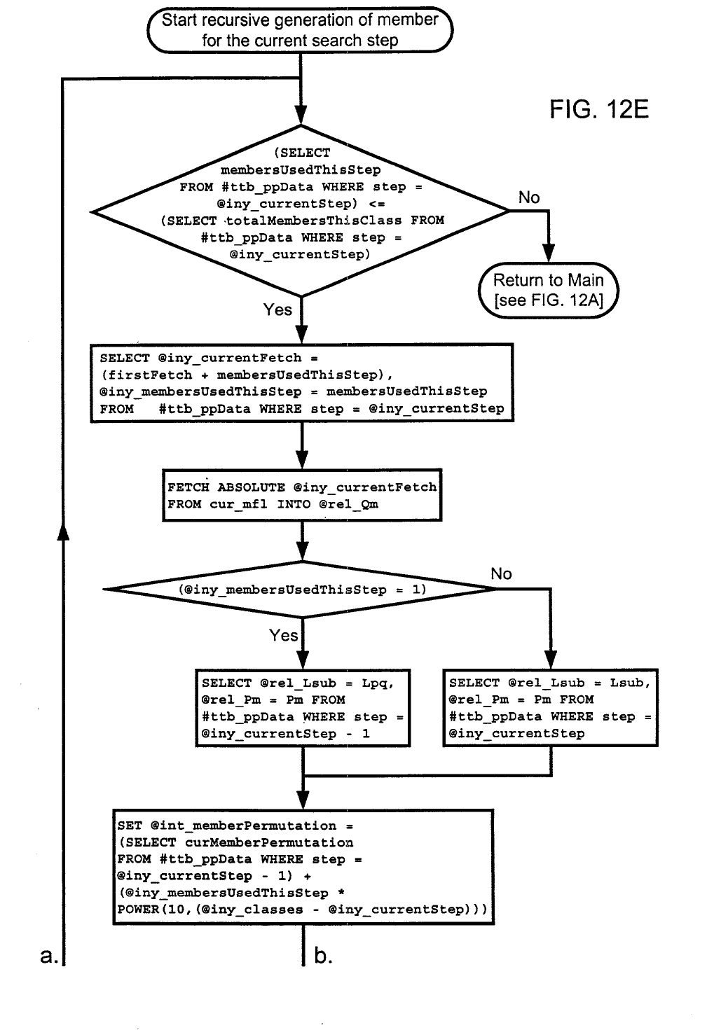

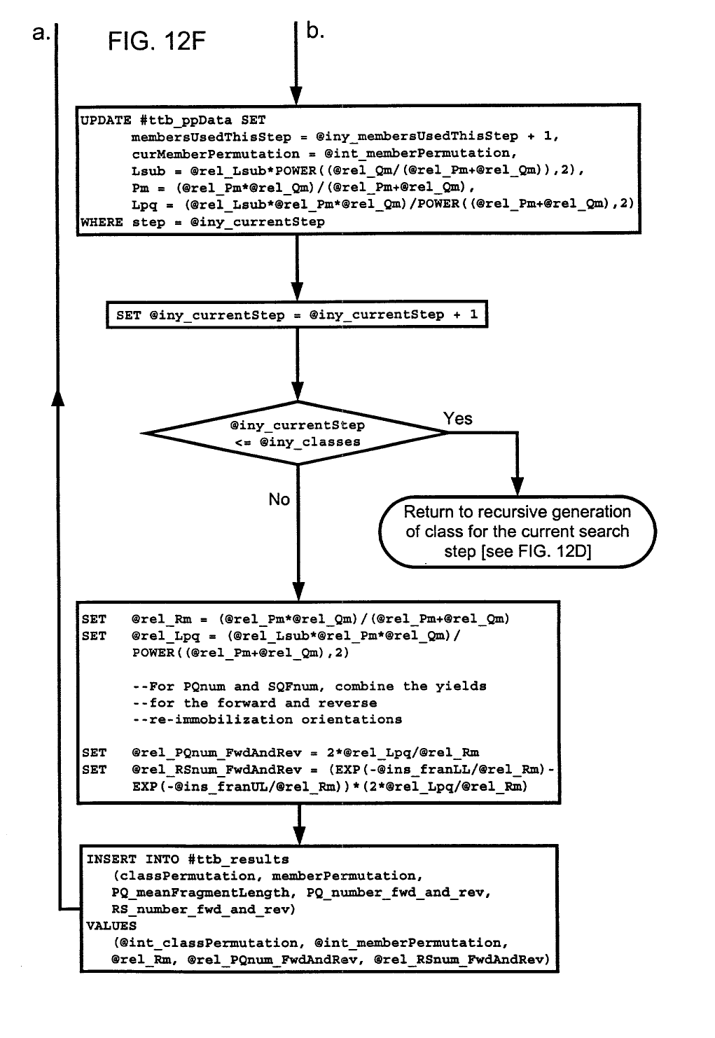

In another aspect, the present invention provides a rapid computational simulation method that is useful for estimating the values of various parameters, such as the average length and total number of SQFs, that would be obtained in an SQF analysis using a search target-group and a primary dataset (Dpₓ) of known or projected size (FIG. 12; see also Examples Tables 22 and 23). In addition to the utility of the simulation method for estimation purposes, it is also useful for guiding the design of search target-groups that may later be used for a full computational or laboratory embodiment of the present invention for the SQF analysis of (Dpₓ) or physical samples of polynucleotides that (Dpₓ) describes.

The computational SQF analysis simulation method includes computer program code, algorithms, data structures and the like, for rapidly estimating the expected number and mean fragment length of all possible SQFs, and the expected number of all possible SQFs whose lengths fall within a size range defined by a “fragment analysis lower limit” (fran_{LL}) and a “fragment analysis upper limit” (fran_{UL}). This method, for the purposes of illustration, is defined as a simulated SQF analysis (sU) using a search target-group (Gu) with (Mᵤ) major classes of major search targets. Each major search class (Cᵢ) in (Gu) contains a limited number of ranked members (Qm_{i,j}) (where for a given major search class Cᵢ, Qmᵢ₁ is the highest-ranked member, Qmᵢ₂ is the second highest-ranked member, and so on). The number (Jmaxᵢ) of members (jᵢ) per major search class (Cᵢ) may vary for each major search class defined by (Gu). Each non-initial search step for a major search class Cᵢ may preferably proceed with the opposite search polarity of the previous search step. The execution of (sU) requires a primary dataset (Dp) as defined earlier, but where the number of strings in Dp may be zero. The only information required for the execution of sU is the mean recurrence length (or mean fragment length, m) in the dataset (Dp), or an estimate of m in Dp, associated with each search target (Q) in Gu, and an initial value for L_{sub}, the total “substrate” length of all of the strings in the dataset (Dp), where L_{sub} may be either the actual size of the primary dataset (Dp) (i.e., the total number of characters in all of the strings in Dp) in a relational database application such as described above, or a projected size for Dp. The method is comprised of the following steps:

a) making the following assumptions, adapted from Bishop et al. (1983) Am. J. Hum. Genet. 35, 795-815, concerning the occurrences of each search target (Q) in (Gu) in the dataset (Dp);

(i) the distribution of the random variable (Y) for the distance between two consecutive occurrences of a search target (Q) is approximated best by the exponential distribution; and therefore, where (m) is the mean recurrence length (or mean fragment length) associated with a search target (Q), and exp(x) is the exponential function raising (e), the base of natural logarithms, to the power (x); then the (probability) Prob [Y>y]=exp(−y/m) for any y≧0; and thus Prob [Y≧y₁]=Prob[Y>(y₁−1)] for integer values of y₁≧1; and by definition Prob [Y≧y₁]=Prob [y₁≦Y≦y₂]+Prob[Y>y₂] for integer values of y₁≧1 and y₂>y₁; and thus Prob [y₁≦Y≦y₂]=Prob[Y>(y₁−1)]−Prob [Y>y₂] for integer values of y₁≧1 and y₂>y₁; and thus Prob [y₁≦Y≦y₂]=exp(−(y₁−1)/m)−exp(−y₂/m) for integer values of y₁≧1 and y₂>y₁;

(ii) if two distinct search targets, (Q₁) and (Q₂), with mean recurrence lengths (m₁) and (m₂), respectively, are used to generate fragments from a region of (Dp), then the expected value E(Rₘ) for the mean length of the resulting fragments is E(Rₘ)=(m₁*m₂)/(m₁+m₂), and the distribution of (Rₘ) remains exponential for the lengths of the fragments regardless of their termini;

(iii) if two distinct search targets, (Q₁) and (Q₂), with mean recurrence lengths (m₁) and (m₂), respectively, are used to generate fragments from a region of (Dp), then the proportion of fragments with only (Q₁) termini is [m₂/(m₁+m₂)]²;

(iv) if two distinct search targets, (Q₁) and (Q₂), with mean recurrence lengths (m₁) and (m₂), respectively, are used to generate fragments from a region of (Dp), then the proportion of fragments with only (Q₂) termini is [m₁/(m₁+m₂)]²;

(v) if two distinct search targets, (Q₁) and (Q₂), with mean recurrence lengths (m₁) and (m₂), respectively, are used to generate fragments from a region of (Dp), then the proportion of fragments with (Q₁ and Q₂) termini is [2*(m₁*m₂)]/[(m₁+m₂)²]; and

b) generating (Mᵤ factorial) permutations of the (Mᵤ) major classes of search targets defined in (Gu), where each class-permutation (CPₖ, where k=1, 2, . . . , Mᵤ!) will be processed as described in the following parts of this claim;

c) subjecting each class-permutation (CPₖ) to a hierarchically ordered, recursive, branching sequence of processing steps defined by the major search class permutation. The initial value for (L_{sub}) is doubled if the strings in the primary dataset (Dp) used in the simulated SQF analysis (sU) represent polynucleotide sequences or entities represented by polynucleotide sequences. This is done because there are two initial search step polarities that may be pursued. For the purposes of defining the recursive sequence of steps, the initial current major search class index (i) is initialized to zero at the very start of the following sequence of steps, and the value of the “petite fragment length” parameter (mₚ) is initialized to the value of the mean recurrence length associated with the partition search target (Qa) in (Dp). Also, for each major search class index (i), Jmaxᵢ is the maximum number of search target members in the major search class (Cᵢ), where the index value i=1, 2, . . . , Mᵤ as defined by (Gu). The hierarchically ordered, recursive, branching sequence of processing steps is comprised of (FIG. 12);

i) if (i)=(Mᵤ−1), typically one would double the size of (L_{sub}) if the strings in the primary dataset (Dp) used in the simulated SQF analysis (sU) represent polynucleotide sequences or entities represented by polynucleotide sequences. This step is analogous to the elution, amplification, and reimmobilization steps described above for the amplification procedure during the laboratory SQF analysis;

ii) if (i)<Mᵤ then increment (i) by one to specify the current major search class (Cᵢ); otherwise the processing steps of the current branch of the recursive procedure are complete;

iii) initialization of the value of the class member index (jᵢ) for the current class to zero;

iv) the designation of the current set of fragments as (PP) fragments, each of which has only “generic” (P) termini, where the two (P) termini on a (PP) fragment may have been generated by different search targets (Q), but are nevertheless classified generically as (P) termini solely because they were generated by search targets that differ from any of the search targets (Qm_{i,j}) in the current major search class (Cᵢ);

v) if (jᵢ)<Jmaxᵢ for the current major class, then increment (jᵢ) by one to specify the current major search target (Qm_{i,j}) for the following steps; otherwise the processing steps are complete for the current major search class (Cᵢ);

vi) use the current major search target (Qm_{i,j}), with mean recurrence length (m_{i,j}), to generate fragments from the current set of (PP) fragments of total length (L_{sub}) with mean fragment length (mₚ). The expected value E(Rₘ)=(mₚ*m_{i,j})/(mₚ+m_{i,j}) for the resulting fragments, and the resulting fragments of interest are of two types: newly generated (PQ) fragments of the correct polarity (i.e., only half of all of the newly generated PQ fragments), each of which has (P and Qm_{i,j}) termini; and unaffected (PP) fragments, each of which has only (P) termini. The total length (L_{pq}) of (PQ) fragments of the correct polarity is estimated as (L_{pq})=(L_{sub})*[(mₚ*m_{i,j})]/[(mₚ+m_{i,j})²]; and the total length (Lₚₚ) of unaffected (PP) fragments is estimated as (Lₚₚ)=(L_{sub})*[m_{i,j}(mₚ+m_{i,j)]}2;

vii) if (i)<Mᵤ, calling of the recursive procedure, initiating a new branch of execution at Step (i) using the newly generated (PQ) fragments obtained in Step (vi) of the current iteration of the current branch of execution, where the new value of (L_{sub})=(L_{pq}) and the new value of (mₚ)=E(Rₘ) where (L_{pq}) and E(Rₘ) are defined in Step (vi) of the current iteration of the current branch of execution. Otherwise if (i)=Mᵤ, the values of various parameters for each process-pattern are stored in a temporary or permanent database table that includes non-nullable data fields or data field combinations for the various parameters, which include: the identity of each process-pattern; E(Rₘ), used as a mean fragment length parameter for all of the SQFs defined by the process-pattern; an estimate of the number (PQₙᵤₘ) of SQFs (of any size) defined by the process-pattern, where (PQₙᵤₘ)=(L_{pq})/E(Rₘ); and an estimate of the number (RSₙᵤₘ) of “ranged” SQFs of length (Iᵣₛ) where (fran_{LL})≦(Iᵣₛ)≦(fran_{UL}), and that are defined by the process-pattern, where (RSₙᵤₘ)=(PQₙᵤₘ)*[exp{−(fran_{LL}−1)/E(Rₘ)}−exp{−fran_{UL}/E(Rₘ)}]; and where (L_{pq}) and E(Rₘ) are defined in Step (vi) of the current iteration of the current branch of execution;

viii) begin the next iteration of fragmentation at Step (v) of the current branch of execution, using the unaffected (PP) fragments as defined in Step (vi) of the current iteration of the current branch of execution, and where the new value of (L_{sub})=(Lₚₚ) and the new value of (mₚ)=E(Rₘ) where (Lₚₚ) and E(Rₘ) are defined in Step (vi) of the current iteration of the current branch of execution; and

d) through the repeated use of the protocol described in Part (c), obtaining for each major class-permutation (CPₖ) the exponential generation of a total of (ΠJmaxᵢ) process-patterns and theoretical “last-generation” SQF-fractions, where each of the fraction of SQFs will be estimated to contain a non-zero real number of SQFs defined by the same process-pattern. The information acquired and stored in the database table or the equivalent described in Step vii of Part (c) above may be queried to determine the results of the simulation for a specific process-pattern or for aggregates thereof.

### EXAMPLES

The following examples describe and illustrate the methods and compositions of the invention. These examples are intended to be merely illustrative of the present invention, and not limiting thereof in either scope or spirit.

### Example 1

Primary datasets of Polynucleotide sequence data.

The data-import software program (see FIGS. 3 and 4) and the relational database application that were developed for a preferred embodiment of the present invention were used to import the primary datasets of polynucleotide sequence data indicated in Tables 1 and 2.

### Example 2

Search targets, target-strings, and target-groups.

The examples of SQF analyses described below make use of several different search target-groups. Table 3 provides information regarding the individual search targets, and their search target-strings, that were used to assemble these search target-groups. All of these search targets are the recognition sequences of the indicated restriction endonucleases, and possess the cut-offset properties indicated. Tables 4 and 5 show the partition (Qa or simply “A”-class) search targets, and major search targets, respectively, that comprise the indicated search target-groups.

### Example 3

Search target mean fragment length data.

Table 6 shows the mean fragment length (or mean recurrence length) data of the indicated search targets and primary datasets referred to in this application. This data is conveniently obtained from the relational database application developed for a preferred embodiment of the present invention.

### Example 4

The coding table for the second-order substrings.

Table 7 shows two related excerpts from the table “tb_hexanucleotide” used as a coding table in the relational database application developed for a preferred embodiment of the present invention. The first column (“Hexanucleotide ID”; formally, “uid_hexanucleotide”) is the primary key of the coding table (see also FIG. 2D).

### Example 5

The storage of input strings as relational records.

Tables 8-10 show how input-strings may be stored in the relational database application developed for a preferred embodiment of the present invention. Note the difference between the ending of a stored circular sequence (Table 9) and a stored linear sequence (Table 10).

### Example 6

SQF analyses of genomic DNA sequence data.

Tables 11-21 document various aspects of SQF analyses of the primary datasets described above (see Tables 1 and 2). Table 11 introduces the various SQF analyses whose results are described in detail in the summaries shown in Tables 17-21. Table 12 describes two equivalent notational schemes used to construct the self-documenting definitions of process-patterns. Tables 13-16 provide an example of a logical sequence of information retrieval from the relational database application developed for a preferred embodiment of the present invention. ACP fragments present in a sequence of interest (e.g., see Table 13) may be first identified by querying the database application. The process-patterns entities present in one or more ACP fragments in the sequence of interest may then be examined as shown in Table 14. Then, one or more specific process-pattern definitions of interest may be used to locate other process-patterns entities in other datasets, as shown in Table 16. Finally, Table 15 provides a step-by-step account of the discovery of process-patterns entities in one of the ACP fragments introduced in Tables 13 and 14.

### Example 7

Simulated SQF analyses. Tables 22 and 23 show the

results of some simulated SQF analyses (see also FIG. 12) of two of the primary datasets described above, and comparisons of these theoretical results with the observed results obtained by actual searching of the datasets as described in FIGS. 6 and 7. Despite the vast difference (926 vs. 12 Mb) in size between the two primary datasets used for these simulated SQF analyses, the results obtained are still useful in both cases for predictive or approximation purposes.

TABLE 1
Primary datasets referred to in this application¹.

| | Human | Human | Mouse | Fruit-fly² | Nematode³ | Yeast⁴ |
|---|---|---|---|---|---|---|
| Dataset ID | 1 | 2 | 3 | 5 | 7 | 9 |
| (primary key) | | | | | | |
| Species | H. sapiens | H. sapiens | M. musculus | D. melanogaster | C. elegans | S. cerevisiae |
| Genome type | Nuclear | Mitochondrion | Nuclear | Nuclear | Nuclear | Nuclear |
| Sequence type | HTG phase 3 | Circular | HTG phase 3 | Gapped | Gapped | Whole |
| | (“finished”) | sequence | (“finished”) | chromosome | chromosome | chromosomes |
| | | | | arms | arms | |
| Dataset provider | NCBI | NCBI | NCBI | U. C. Berkeley | NCBI | Stanford Univ. |
| Sequences count | 8,639 | 1 | 142 | 6 | 6 | 16 |
| Total nt. | 926,000,944 | 16,569 | 16,379,018 | 116,117,226 | 100,096,025 | 12,069,247 |
| Degenerate (N) nt. | 40,923 | 0 | 894 | 1,915,658 | 4,868,144 | 0 |

(1) Datasets are comprised of genomic DNA sequences of the indicated type.
(2) Dataset 5 (March 2000 release) was downloaded from the Berkeley Drosophila Genome Database FTP site on Oct. 4, 2000.
(3) Dataset 7 was downloaded from the NCBI FTP site on Oct. 4, 2000.
(4) Dataset 9 was downloaded from the Saccharomyces Genome Database FTP site on Oct. 4, 2000.

TABLE 2
Acquisition specifications of NCBI datasets
referred to in the application^{1,2}.

| Dataset ID | |
|---|---|
| (primary | |
| key) | URL used with “PmQty” to obtain GenBank GIs |
| 1 | http://www.ncbi.nlm.nih.gov/entrez/utils/pmqty.fcgi?&db= |
| | n&term=((gbdiv+pri[PROP])+AND+(Homo+ |
| | sapiens[ORGN])+AND+(biomol+genomic[PROP])+AND+ |
| | (htgs+phase3[PROP]))&dispmax=999999999&mode=html |
| 2 | http://www.ncbi.nlm.nih.gov/entrez/utils/pmqty.fcgi?&db= |
| | n&term=(NC_001807[ACCN])&dispmax= |
| | 999999999&mode=html |
| 3 | http://www.ncbi.nlm.nih.gov/entrez/utils/pmqty.fcgi?&db= |
| | n&term=((Mus+musculus[ORGN])+AND+(biomol+ |
| | genomic[PROP])+AND+(htgs+phase3[PROP]))&dispmax= |
| | 999999999&mode=html |

¹URL: uniform resource locator; PmQty is the name of an NCBI web application utility for downloading GIs (GenBank identifiers) and is described at http://www.ncbi.nlm.nih.gov/entrez/utils/pmqty_help.html.
²Each dataset contained all of the relevant publicly available sequence data that was available as of Nov. 12, 2000.

TABLE 3
Data for search targets and search target-strings referred to in this application¹.

| Target name | T ID | TS ID | Is pal. | TargetString | CO_{ds} | CO_{rc} | Pst-R | Pst-L | Pre-R | Pre-L | Len | SQL-hx0 | SQL-hx1 | SQL-hx2 |
|---|---|---|---|---|---|---|---|---|---|---|---|---|---|---|
| SspI | 1 | 1 | Yes | AATATT | 2 | 3 | 2 | 3 | 5 | 0 | 6 | AATATT | <NULL> | <NULL> |
| Acc65I | 2 | 2 | Yes | GGTACC | 0 | 5 | 4 | 1 | 5 | 0 | 6 | GGTACC | <NULL> | <NULL> |
| PaeI | 3 | 3 | Yes | GCATGC | 4 | 1 | 4 | 1 | 5 | 0 | 6 | GCATGC | <NULL> | <NULL> |
| AflII | 4 | 4 | Yes | CTTAAG | 0 | 5 | 4 | 1 | 5 | 0 | 6 | CTTAAG | <NULL> | <NULL> |
| StuI | 5 | 5 | Yes | AGGCCT | 2 | 3 | 2 | 3 | 5 | 0 | 6 | AGGCCT | <NULL> | <NULL> |
| BstEII | 6 | 6 | Yes | GGTNACC | 0 | 6 | 5 | 1 | 6 | 0 | 7 | GGT_AC | C % | <NULL> |
| MfeI | 7 | 7 | Yes | CAATTG | 0 | 5 | 4 | 1 | 5 | 0 | 6 | CAATTG | <NULL> | <NULL> |
| AvrII | 8 | 8 | Yes | CCTAGG | 0 | 5 | 4 | 1 | 5 | 0 | 6 | CCTAGG | <NULL> | <NULL> |
| HindIII | 9 | 9 | Yes | AAGCTT | 0 | 5 | 4 | 1 | 5 | 0 | 6 | AAGCTT | <NULL> | <NULL> |
| Bsh1365I | 10 | 10 | Yes | GATNNNNATC | 4 | 5 | 4 | 5 | 9 | 0 | 10 | GAT___ | _ATC % | <NULL> |
| ScaI | 11 | 11 | Yes | AGTACT | 2 | 3 | 2 | 3 | 5 | 0 | 6 | AGTACT | <NULL> | <NULL> |
| Bpu1102I | 12 | 12 | Yes | GCTNAGC | 1 | 5 | 4 | 2 | 6 | 0 | 7 | GCT_AG | C % | <NULL> |
| BsrGI | 13 | 13 | Yes | TGTACA | 0 | 5 | 4 | 1 | 5 | 0 | 6 | TGTACA | <NULL> | <NULL> |
| SpeI | 14 | 14 | Yes | ACTAGT | 0 | 5 | 4 | 1 | 5 | 0 | 6 | ACTAGT | <NULL> | <NULL> |
| Cfr9I | 15 | 15 | Yes | CCCGGG | 0 | 5 | 4 | 1 | 5 | 0 | 6 | CCCGGG | <NULL> | <NULL> |
| BcII | 16 | 16 | Yes | TGATCA | 0 | 5 | 4 | 1 | 5 | 0 | 6 | TGATCA | <NULL> | <NULL> |
| NcoI | 17 | 17 | Yes | CCATGG | 0 | 5 | 4 | 1 | 5 | 0 | 6 | CCATGG | <NULL> | <NULL> |
| BamHI | 18 | 18 | Yes | GGATCC | 0 | 5 | 4 | 1 | 5 | 0 | 6 | GGATCC | <NULL> | <NULL> |
| Eco32I | 19 | 19 | Yes | GATATC | 2 | 3 | 2 | 3 | 5 | 0 | 6 | GATATC | <NULL> | <NULL> |
| BgIII | 20 | 20 | Yes | AGATCT | 0 | 5 | 4 | 1 | 5 | 0 | 6 | AGATCT | <NULL> | <NULL> |
| XbaI | 21 | 21 | Yes | TCTAGA | 0 | 5 | 4 | 1 | 5 | 0 | 6 | TCTAGA | <NULL> | <NULL> |
| AseI | 22 | 22 | Yes | ATTAAT | 1 | 4 | 3 | 2 | 5 | 0 | 6 | ATTAAT | <NULL> | <NULL> |
| NdeI | 23 | 23 | Yes | CATATG | 1 | 4 | 3 | 2 | 5 | 0 | 6 | CATATG | <NULL> | <NULL> |
| SacI | 24 | 24 | Yes | GAGCTC | 4 | 1 | 4 | 1 | 5 | 0 | 6 | GAGCTC | <NULL> | <NULL> |
| BssSI | 26 | −26 | No | CACGAG | 0 | 5 | 4 | 1 | 5 | 0 | 6 | CACGAG | <NULL> | <NULL> |
| BssSI | 26 | 26 | No | CTCGTG | 0 | 5 | 4 | 1 | 5 | 0 | 6 | CTCGTG | <NULL> | <NULL> |
| HpyCH4 IV | 31 | 31 | Yes | ACGT | 0 | 3 | 2 | 1 | 3 | 0 | 4 | ACGT % | <NULL> | <NULL> |
| MspI | 32 | 32 | Yes | CCGG | 0 | 3 | 2 | 1 | 3 | 0 | 4 | CCGG % | <NULL> | <NULL> |

(1) Legend: T ID, target ID; TS ID, target-string ID; Is pal., is the target a palindrome; CO, cut-off; ds, dataset strand; rc, reverse complement strand; Pst, post-cut; Pre, pre-cut; R, right; L, left; Len, length (nt.); SQL-hx(0, 1, 2), SQL LIKE operator clause.

TABLE 4
Search target groups, and their partition search targets,
that are referred to in this application¹.

| Target | Partition target | Qa |
|---|---|---|
| group ID | (Qa) name | target ID |
| 1 | SspI | 1 |
| 2 | AseI | 22 |
| 3 | NdeI | 23 |
| 4 | SacI | 24 |
| 5 | BssSI | 26 |
| 6 | HpyCH4 IV | 31 |
| 7 | Msp I | 32 |

¹In all cases, the Qa search target polarity used was zero.

TABLE 5
The major search targets that were used for all of the
search target-groups referred to in this application.

| | | Member | | |
|---|---|---|---|---|
| Class | Class | number | Target | Target |
| number | code | (rank) | name | ID |
| 1 | B | 1 | Acc65I | 2 |
| 1 | B | 2 | PaeI | 3 |
| 1 | B | 3 | AfIII | 4 |
| 1 | B | 4 | StuI | 5 |
| 2 | C | 1 | BstEII | 6 |
| 2 | C | 2 | MfeI | 7 |
| 2 | C | 3 | AvrII | 8 |
| 2 | C | 4 | HindIII | 9 |
| 3 | D | 1 | Bsh1365I | 10 |
| 3 | D | 2 | ScaI | 11 |
| 3 | D | 3 | Bpu1102I | 12 |
| 3 | D | 4 | BsrGI | 13 |
| 4 | E | 1 | SpeI | 14 |
| 4 | E | 2 | Cfr9I | 15 |
| 4 | E | 3 | BcII | 16 |
| 4 | E | 4 | NcoI | 17 |
| 5 | F | 1 | BamHI | 18 |
| 5 | F | 2 | Eco32I | 19 |
| 5 | F | 3 | BgIII | 20 |
| 5 | F | 4 | XbaI | 21 |

TABLE 6
Mean fragment lengths (MFL) obtained using the indicated datasets and search targets¹.

| Target | Target | Code | Dataset 1 | Dataset 3 | Dataset 5 | Dataset 7 | Dataset 9 |
|---|---|---|---|---|---|---|---|
| ID | name | [TG ID] | (human) | (mouse) | (fruit-fly) | (nematode) | (yeast) |
| 1 | SspI | A [1] | 1248 | 2086 | 1064 | 796 | 1050 |
| 22 | AseI | A [2] | 1976 | 2865 | 1454 | 1614 | 1787 |
| 23 | NdeI | A [3] | 3130 | 3316 | 3339 | 5072 | 3581 |
| 24 | SacI | A [4] | 4438 | 3359 | 5247 | 5038 | 8467 |
| 26 | BssSI | A [5] | 6473 | 7345 | 4266 | 4475 | 5932 |
| 31 | HpyCH4 IV | A [6] | 1297 | 1374 | 513 | 472 | 411 |
| 32 | Msp I | A [7] | 1155 | 1198 | 504 | 702 | 871 |
| 2 | Acc65I | B1 | 8847 | 6682 | 18140 | 14149 | 6330 |
| 3 | PaeI | B2 | 4847 | 3846 | 4574 | 10432 | 7972 |
| 4 | AflII | B3 | 4269 | 4478 | 4365 | 10692 | 6143 |
| 5 | StuI | B4 | 3319 | 2842 | 9542 | 13140 | 8351 |
| 6 | BstEII | C1 | 7427 | 5922 | 8073 | 14428 | 7739 |
| 7 | MfeI | C2 | 5017 | 5662 | 2201 | 2069 | 2097 |
| 8 | AvrII | C3 | 4511 | 3839 | 22749 | 17402 | 17584 |
| 9 | HindIII | C4 | 3352 | 3300 | 3666 | 2667 | 2707 |
| 10 | Bsh1365I | D1 | 6065 | 6649 | 3962 | 4233 | 3531 |
| 11 | ScaI | D2 | 5043 | 3900 | 6624 | 4778 | 3939 |
| 12 | Bpu1102I | D3 | 4879 | 3278 | 4586 | 11603 | 10018 |
| 13 | BsrGI | D4 | 3375 | 3046 | 3349 | 4211 | 3989 |
| 14 | SpeI | E1 | 6748 | 7073 | 9661 | 7704 | 5047 |
| 15 | Cfr9I | E2 | 6072 | 8555 | 13542 | 30524 | 37514 |
| 16 | BcII | E3 | 3773 | 4194 | 4943 | 3318 | 3048 |
| 17 | NcoI | E4 | 3533 | 2830 | 4924 | 10226 | 5018 |
| 18 | BamHI | F1 | 7013 | 4328 | 5938 | 8821 | 7161 |
| 19 | Eco32I | F2 | 6246 | 6232 | 5329 | 4638 | 2856 |
| 20 | BgIII | F3 | 3590 | 2625 | 5542 | 4713 | 3399 |
| 21 | XbaI | F4 | 3476 | 3196 | 8014 | 3552 | 4223 |

(1) MFL values are in nt. In all cases, the target polarity was zero. Legend: TG, target-group; “Code” is the class + member code.

TABLE 7
Excerpts from the second-order substring coding table (“tb_hexanucleotide”)
referred to in this application¹.

| Hexanucleotide | | Dipeptide | Dipeptide | Hexanucleotide | DipeptideRevComp | DipeptideRevComp |
|---|---|---|---|---|---|---|
| ID | Hexanucleotide | 1charCode | 3charCode | RevComp | 1charCode | 3charCode |
| −6012 | ACTTTT | TF | ThrPhe | AAAAGT | KS | LysSer |
| −6011 | CCTTTT | PF | ProPhe | AAAAGG | KR | LysArg |
| −6010 | GCTTTT | AF | AlaPhe | AAAAGC | KS | LysSer |
| −6009 | TCTTTT | SF | SerPhe | AAAAGA | KR | LysArg |
| −6008 | AGTTTT | SF | SerPhe | AAAACT | KT | LysThr |
| −6007 | CGTTTT | RF | ArgPhe | AAAACG | KT | LysThr |
| −6006 | GGTTTT | GF | GlyPhe | AAAACC | KT | LysThr |
| −6005 | TGTTTT | CF | CysPhe | AAAACA | KT | LysThr |
| −6004 | ATTTTT | IF | IlePhe | AAAAAT | KN | LysAsn |
| −6003 | CTTTTT | LF | LeuPhe | AAAAAG | KK | LysLys |
| −6002 | GTTTTT | VF | ValPhe | AAAAAC | KN | LysAsn |
| −6001 | TTTTTT | FF | PhePhe | AAAAAA | KK | LysLys |
| 6001 | AAAAAA | KK | LysLys | TTTTTT | FF | PhePhe |
| 6002 | AAAAAC | KN | LysAsn | GTTTTT | VF | ValPhe |
| 6003 | AAAAAG | KK | LysLys | CTTTTT | LF | LeuPhe |
| 6004 | AAAAAT | KN | LysAsn | ATTTTT | IF | IlePhe |
| 6005 | AAAACA | KT | LysThr | TGTTTT | CF | CysPhe |
| 6006 | AAAACC | KT | LysThr | GGTTTT | GF | GlyPhe |
| 6007 | AAAACG | KT | LysThr | CGTTTT | RF | ArgPhe |
| 6008 | AAAACT | KT | LysThr | AGTTTT | SF | SerPhe |
| 6009 | AAAAGA | KR | LysArg | TCTTTT | SF | SerPhe |
| 6010 | AAAAGC | KS | LysSer | GCTTTT | AF | AlaPhe |
| 6011 | AAAAGG | KR | LysArg | CCTTTT | PF | ProPhe |
| 6012 | AAAAGT | KS | LysSer | ACTTTT | TF | ThrPhe |

(1) Legend: RevComp, reverse complementary strand.

TABLE 8
The storage of input string data as relational records: The beginning of
an encoded circular polynucleotide sequence^{1,2}.

| Position | uid_hx0 | uid_hx1 | uid_hx2 | hx0 | hx1 | hx2 |
|---|---|---|---|---|---|---|
| 1 | 7619 | −6498 | 6747 | GATCAC | AGGTCT | ATCACC |
| 2 | 6746 | 7816 | −7805 | ATCACA | GGTCTA | TCACCC |
| 3 | −7485 | −6718 | −6625 | TCACAG | GTCTAT | CACCCT |
| 4 | 6944 | −7615 | 6333 | CACAGG | TCTATC | ACCCTA |
| 5 | 6288 | 7416 | −6726 | ACAGGT | CTATCA | CCCTAT |
| 6 | 7015 | −7868 | −6197 | CAGGTC | TATCAC | CCTATT |
| 7 | −6498 | 6747 | 7420 | AGGTCT | ATCACC | CTATTA |
| 8 | 7816 | −7805 | 7948 | GGTCTA | TCACCC | TATTAA |
| 9 | −6718 | −6625 | 6842 | GTCTAT | CACCCT | ATTAAC |
| 10 | −7615 | 6333 | −7822 | TCTATC | ACCCTA | TTAACC |
| 11 | 7416 | −6726 | 7902 | CTATCA | CCCTAT | TAACCA |
| 12 | −7868 | −6197 | 6081 | TATCAC | CCTATT | AACCAC |
| 13 | 6747 | 7420 | 6313 | ATCACC | CTATTA | ACCACT |
| 14 | −7805 | 7948 | 7092 | TCACCC | TATTAA | CCACTC |
| 15 | −6625 | 6842 | 6974 | CACCCT | ATTAAC | CACTCA |
| 16 | 6333 | −7822 | 6436 | ACCCTA | TTAACC | ACTCAC |
| 17 | −6726 | 7902 | −7371 | CCCTAT | TAACCA | CTCACG |
| 18 | −6197 | 6081 | −7193 | CCTATT | AACCAC | TCACGG |
| 19 | 7420 | 6313 | 6968 | CTATTA | ACCACT | CACGGG |
| 20 | 7948 | 7092 | 6401 | TATTAA | CCACTC | ACGGGA |
| 21 | 6842 | 6974 | 7335 | ATTAAC | CACTCA | CGGGAG |
| 22 | −7822 | 6436 | −7726 | TTAACC | ACTCAC | GGGAGC |
| 23 | 7902 | −7371 | −6578 | TAACCA | CTCACG | GGAGCT |
| 24 | 6081 | −7193 | 95 | AACCAC | TCACGG | GAGCTC |
| 25 | 6313 | 6968 | −6512 | ACCACT | CACGGG | AGCTCT |

(1) Legend: uid_hx(0, 1, 2), unique identifier for the encoded, ordered (0, 1, 2) hexanucleotides (hx0, hx1, hx2).
(2) The excerpt shown is from the 16,569-nt. long sequence (GenBank Accession ID NC_001807) of the mitochondrion of Homo sapiens, which comprises dataset 2.

TABLE 9
The storage of input string data as relational records: The end of an
encoded circular polynucleotide sequence^{1, 2}.

| Position | uid_hx0 | uid_hx1 | uid_hx2 | hx0 | hx1 | hx2 |
|---|---|---|---|---|---|---|
| 16545 | −6620 | 7900 | 6494 | TCCCCT | TAAATA | AGACAT |
| 16546 | −6167 | 6049 | 7559 | CCCCTT | AAATAA | GACATC |
| 16547 | 7154 | 6189 | 6296 | CCCTTA | AATAAG | ACATCA |
| 16548 | 7227 | 6698 | 7039 | CCTTAA | ATAAGA | CATCAC |
| 16549 | 7489 | −7863 | 6748 | CTTAAA | TAAGAC | ATCACG |
| 16550 | −6877 | 6130 | 7956 | TTAAAT | AAGACA | TCACGA |
| 16551 | 7900 | 6494 | −6779 | TAAATA | AGACAT | CACGAT |
| 16552 | 6049 | 7559 | 6378 | AAATAA | GACATC | ACGATG |
| 16553 | 6189 | 6296 | −7109 | AATAAG | ACATCA | CGATGG |
| 16554 | 6698 | 7039 | 7629 | ATAAGA | CATCAC | GATGGA |
| 16555 | −7863 | 6748 | −6758 | TAAGAC | ATCACG | ATGGAT |
| 16556 | 6130 | 7956 | −7620 | AAGACA | TCACGA | TGGATC |
| 16557 | 6494 | −6779 | 7761 | AGACAT | CACGAT | GGATCA |
| 16558 | 7559 | 6378 | 7619 | GACATC | ACGATG | GATCAC |
| 16559 | 6296 | −7109 | 6746 | ACATCA | CGATGG | ATCACA |
| 16560 | 7039 | 7629 | −7485 | CATCAC | GATGGA | TCACAG |
| 16561 | 6748 | −6758 | 6944 | ATCACG | ATGGAT | CACAGG |
| 16562 | 7956 | −7620 | 6288 | TCACGA | TGGATC | ACAGGT |
| 16563 | −6779 | 7761 | 7015 | CACGAT | GGATCA | CAGGTC |
| 16564 | 6378 | 7619 | −6498 | ACGATG | GATCAC | AGGTCT |
| 16565 | −7109 | 6746 | 7816 | CGATGG | ATCACA | GGTCTA |
| 16566 | 7629 | −7485 | −6718 | GATGGA | TCACAG | GTCTAT |
| 16567 | −6758 | 6944 | −7615 | ATGGAT | CACAGG | TCTATC |
| 16568 | −7620 | 6288 | 7416 | TGGATC | ACAGGT | CTATCA |
| 16569 | 7761 | 7015 | −7868 | GGATCA | CAGGTC | TATCAC |

(1) Legend: uid_hx(0, 1, 2), unique identifier for the encoded, ordered (0, 1, 2) hexanucleotides (hx0, hx1, hx2).
(2) The excerpt shown is from the 16,569-nt. long sequence (GenBank Accession ID NC_001807) of the mitochondrion of Homo sapiens, which comprises dataset 2.

TABLE 10
The storage of input string data as relational records: The end of an
encoded linear polynucleotide sequence^{1, 2}.

| Position | uid_hx0 | uid_hx1 | uid_hx2 | hx0 | hx1 | hx2 |
|---|---|---|---|---|---|---|
| 172 | −7787 | 6513 | 6093 | GTTCCC | AGAGGA | AACCTC |
| 173 | −8005 | 7597 | 6354 | TTCCCA | GAGGAA | ACCTCA |
| 174 | −7477 | 6586 | 7207 | TCCCAG | AGGAAA | CCTCAA |
| 175 | 7120 | 7742 | 7424 | CCCAGA | GGAAAC | CTCAAG |
| 176 | 7095 | 7524 | −7739 | CCAGAG | GAAACC | TCAAGC |
| 177 | 6990 | 6024 | 6918 | CAGAGG | AAACCT | CAAGCG |
| 178 | 6513 | 6093 | 6152 | AGAGGA | AACCTC | AAGCGG |
| 179 | 7597 | 6354 | 6567 | GAGGAA | ACCTCA | AGCGGA |
| 180 | 6586 | 7207 | 5433 | AGGAAA | CCTCAA | GCGGA |
| 181 | 7742 | 7424 | 4081 | GGAAAC | CTCAAG | CGGA |
| 182 | 7524 | −7739 | 3029 | GAAACC | TCAAGC | GGA |
| 183 | 6024 | 6918 | 2006 | AAACCT | CAAGCG | GA |
| 184 | 6093 | 6152 | 1001 | AACCTC | AAGCGG | A |
| 185 | 6354 | 6567 | 0 | ACCTCA | AGCGGA | |
| 186 | 7207 | 5433 | 0 | CCTCAA | GCGGA | |
| 187 | 7424 | 4081 | 0 | CTCAAG | CGGA | |
| 188 | −7739 | 3029 | 0 | TCAAGC | GGA | |
| 189 | 6918 | 2006 | 0 | CAAGCG | GA | |
| 190 | 6152 | 1001 | 0 | AAGCGG | A | |
| 191 | 6567 | 0 | 0 | AGCGGA | | |
| 192 | 5433 | 0 | 0 | GCGGA | | |
| 193 | 4081 | 0 | 0 | CGGA | | |
| 194 | 3029 | 0 | 0 | GGA | | |
| 195 | 2006 | 0 | 0 | GA | | |
| 196 | 1001 | 0 | 0 | A | | |

(1) Legend: uid_hx(0, 1, 2), unique identifier for the encoded, ordered (0, 1, 2) hexanucleotides (hx0, hx1, hx2).
(2) The excerpt shown is from a 196-nt. long genomic DNA fragment from the human genome (GenBank Accession ID AL031002.1 sequence, which is part of dataset 1).

TABLE 11
SQF analyses of genomic DNA sequence data
referred to in this application¹.

| | SQF analysis ID for the indicated dataset | | | | |
|---|---|---|---|---|---|
| | [dataset ID in parentheses] | | | | |
| Target-group | Human | Mouse | Fruit-fly | Nematode | Yeast |
| ID | [1] | [3] | [5] | [7] | [9] |
| 1 | 1 | 9 | 17 | 26 | 34 |
| 2 | 2 | 10 | 18 | 27 | 35 |
| 3 | 3 | 11 | 19 | 28 | 36 |
| 4 | 4 | 12 | 20 | 29 | 38 |
| 5 | 43 | 59 | 75 | 91 | 107 |
| 6 | 44 | 60 | 76 | 92 | 108 |
| 7 | 45 | 61 | 77 | 93 | 109 |

(1) For these SQF analyses, cut-offsets were not ignored, linear sequences were not pseudo-partitioned, and the site-conflict collar value = 4.

TABLE 12
Two equivalent notational schemes used to describe the 20 first-
generation fractions obtained from a 5 × 4 search target-group.

| Alphanumeric | Numeric |
|---|---|
| /B1; /B2; /B3; /B4 | /Q_{1, 1}; /Q_{1, 2}; /Q_{1, 3}; /Q_{1, 4} |
| /C1; /C2; /C3; /C4 | /Q_{2, 1}; /Q_{2, 2}; /Q_{2, 3}; /Q_{2, 4} |
| /D1; /D2; /D3; /D4 | /Q_{3, 1}; /Q_{3, 2}; /Q_{3, 3}; /Q_{3, 4} |
| /E1; /E2; /E3; /E4 | /Q_{4, 1}; /Q_{4, 2}; /Q_{4, 3}; /Q_{4, 4} |
| /F1; /F2; /F3; /F4 | /Q_{5, 1}; /Q_{5, 2}; /Q_{5, 3}; /Q_{5, 4} |

TABLE 13
Some of the ACP fragments obtained from an SQF analysis¹.

| | | Strand- | | Major | Process- |
|---|---|---|---|---|---|
| Qa-site | Qa-site | polarity | Length | site | pattern |
| 5′-posn. | 3′-posn. | (ACPF) | (nt.) | count | count |
| 298 | 5244 | 0 | 4947 | 22 | 18 |
| 6711 | 7777 | 0 | 1067 | 8 | 4 |
| 11444 | 13508 | 0 | 2065 | 14 | 2 |

(1) ACP fragments were obtained by SQF analysis #1. These results were from a human genomic DNA sequence fragment (GenBank Accession ID Z68758.1).

TABLE 14
Some of the process-patterns (PP) obtained from an SQF analysis¹.

| | | | | | SQF | SQF | |
|---|---|---|---|---|---|---|---|
| | Strand | | Class | Member | length | length | Site |
| Qa-site | polarity | PP | permu- | permu- | (nt.) | (nt.) | con- |
| 5′-posn. | (PP) | index | tation | tation | [O-fip] | [R-fip] | flicts |
| 298 | −1 | −8 | 54231 | 11112 | 953 | 15 | 0 |
| 298 | −1 | −7 | 54132 | 11111 | 965 | 490 | 0 |
| 298 | −1 | −6 | 53241 | 11142 | 464 | 15 | 0 |
| 298 | −1 | −5 | 53142 | 11141 | 476 | 490 | 0 |
| 298 | −1 | −4 | 25134 | 13214 | 489 | 455 | 0 |
| 298 | −1 | −3 | 24153 | 11231 | 945 | 944 | 0 |
| 298 | −1 | −2 | 15234 | 13114 | 489 | 471 | 0 |
| 298 | −1 | −1 | 14253 | 11131 | 945 | 960 | 0 |
| 298 | 1 | 1 | 24135 | 14131 | 909 | 71 | 0 |
| 298 | 1 | 2 | 24531 | 14134 | 537 | 376 | 0 |
| 298 | 1 | 3 | 35412 | 11411 | 490 | 476 | 0 |
| 298 | 1 | 4 | 35421 | 11412 | 15 | 464 | 0 |
| 298 | 1 | 5 | 41523 | 11311 | 960 | 945 | 0 |
| 298 | 1 | 6 | 42513 | 11321 | 944 | 945 | 0 |
| 298 | 1 | 7 | 45312 | 11111 | 490 | 965 | 0 |
| 298 | 1 | 8 | 45321 | 11112 | 15 | 953 | 0 |
| 298 | 1 | 9 | 51243 | 11343 | 180 | 544 | 1 |
| 298 | 1 | 10 | 52314 | 12314 | 0 | 185 | 1 |
| 6711 | −1 | −1 | 41523 | 43242 | 67 | 56 | 0 |
| 6711 | 1 | 1 | 14253 | 34422 | 56 | 67 | 0 |
| 6711 | 1 | 2 | 21345 | 44242 | 190 | 61 | 0 |
| 6711 | 1 | 3 | 21543 | 44244 | 66 | 122 | 0 |
| 11444 | −1 | −1 | 32154 | 24111 | 217 | 254 | 0 |
| 11444 | 1 | 1 | 23514 | 42111 | 254 | 217 | 0 |

¹PP were obtained by SQF analysis #1. These results (see also Table 13) were from a human genomic DNA sequence fragment (GenBank Accession ID Z68758.1).

TABLE 15
The step-wise generation of process-pattern entities from an ACP fragment

| | | | | | Qa | B3 | F3 | C4 | D2 | F2 | D4 | E4 | B4 | Qa |
|---|---|---|---|---|---|---|---|---|---|---|---|---|---|---|
| PP | Class | Mem. | Search | Search | SspI | AflII | BgIII | HindIII | ScaI | Eco321 | BsrGI | NcoI | StuI | SSpI |
| index | perm. | perm. | step | pol. | 6711 | 6958 | 6962 | 7059 | 7124 | 7183 | 7305 | 7376 | 7600 | 7777 |
| −1 | 4 | 4 | 1 | −1 | | | | | | | | E4 | | |
| −1 | 41 | 43 | 2 | +1 | | B3 | | | | | | | | |
| −1 | 415 | 432 | 3 | −1 | | | | | | F2 | | | | |
| −1 | 4152 | 4324 | 4 | +1 | | | | C4 | | | | | | |
| −1 | 41523 | 43242 | 5 | −1 | | | | | D2 | | | | | |
| 1 | 1 | 3 | 1 | +1 | | B3 | | | | | | | | |
| 1 | 14 | 34 | 2 | −1 | | | | | | | | E4 | | |
| 1 | 142 | 344 | 3 | +1 | | | | C4 | | | | | | |
| 1 | 1425 | 3442 | 4 | −1 | | | | | | F2 | | | | |
| 1 | 14253 | 34422 | 5 | +1 | | | | | D2 | | | | | |
| 2 | 2 | 4 | 1 | +1 | | | | C4 | | | | | | |
| 2 | 21 | 44 | 2 | −1 | | | | | | | | | B4 | |
| 2 | 213 | 442 | 3 | +1 | | | | | D2 | | | | | |
| 2 | 2134 | 4424 | 4 | −1 | | | | | | | | E4 | | |
| 2 | 21345 | 44242 | 5 | +1 | | | | | | F2 | | | | |
| 3 | 2 | 4 | 1 | +1 | | | | C4 | | | | | | |
| 3 | 21 | 44 | 2 | −1 | | | | | | | | | B4 | |
| 3 | 215 | 442 | 3 | +1 | | | | | | F2 | | | | |
| 3 | 2154 | 4424 | 4 | −1 | | | | | | | | E4 | | |
| 3 | 21543 | 44244 | 5 | +1 | | | | | | | D4 | | | |

(1) PP were obtained by SQF analysis #1. These results (see also Tables 13 and 14) were from the indicated ACP fragment between positions 6711-7777 in a human genomic DNA sequence fragment (GenBank Accession ID Z68758.1).

TABLE 16
Process-pattern comparisons.

| Ref. or | SQFA | Seq. | Qa-site | Str. pol. | | Class | Member | SQF len | SQF len | Site |
|---|---|---|---|---|---|---|---|---|---|---|
| Match | ID | ID | 5′ | (PP) | PP index | perm. | perm. | [O-fip] | [R-fip] | conflicts |
| Reference | 1 | 30 | 298 | 1 | 8 | 45321 | 11112 | 15 | 953 | 0 |
| match | 17 | 8014 | 1722700 | 1 | 1 | 45321 | 11112 | 27 | 59 | 1 |
| match | 17 | 8014 | 4057792 | −1 | −14 | 45321 | 11112 | 771 | 1639 | 0 |
| match | 17 | 8013 | 4932766 | −1 | −2 | 45321 | 11112 | 703 | 446 | 0 |
| match | 17 | 8017 | 6456409 | −1 | −14 | 45321 | 11112 | 1977 | 74 | 0 |
| match | 17 | 7719 | 8208890 | −1 | −5 | 45321 | 11112 | 212 | 763 | 0 |
| match | 17 | 8015 | 8507985 | 1 | 4 | 45321 | 11112 | 81 | 425 | 0 |
| match | 17 | 8017 | 8877878 | −1 | −5 | 45321 | 11112 | 480 | 243 | 0 |
| match | 17 | 7719 | 17145081 | −1 | −4 | 45321 | 11112 | 188 | 655 | 0 |
| match | 17 | 7719 | 18650690 | −1 | −2 | 45321 | 11112 | 191 | 253 | 0 |
| match | 17 | 8014 | 19007274 | 1 | 13 | 45321 | 11112 | 1629 | 237 | 0 |
| match | 17 | 8015 | 20075237 | 1 | 6 | 45321 | 11112 | 678 | 357 | 0 |
| match | 17 | 7719 | 20467821 | −1 | −12 | 45321 | 11112 | 1376 | 951 | 0 |
| match | 17 | 7719 | 25567362 | 1 | 5 | 45321 | 11112 | 271 | 644 | 0 |
| match | 17 | 7719 | 27100360 | −1 | −4 | 45321 | 11112 | 30 | 195 | 0 |
| match | 17 | 7719 | 27305305 | 1 | 3 | 45321 | 11112 | 342 | 1148 | 0 |

(1) The reference PP was obtained by SQF analysis #1, specifically (see Tables 13 and 14) from an ACP fragment starting at position 298 in a human genomic DNA sequence fragment (GenBank Accession ID Z68758.1). The matching PP were obtained using SQF analysis #17, an SQF analysis of the available genomic DNA sequence from Drosophila.

TABLE 17
Summary results for SQF analyses of human genomic DNA sequence data^{1,2}.

| SQF analysis ID: | 1 | 2 | 3 | 4 | 43 | 44 | 45 |
|---|---|---|---|---|---|---|---|
| Target-group ID | 1 | 2 | 3 | 4 | 5 | 6 | 7 |
| Partition fragments: | | | | | | | |
| Count | 711906 | 441647 | 273148 | 189226 | 122141 | 694780 | 767917 |
| Length (mean, nt.) | 1248 | 1976 | 3130 | 4438 | 6473 | 1297 | 1155 |
| Length (s.d., nt.) | 1746 | 2615 | 3695 | 4966 | 7574 | 1398 | 2003 |
| ACP fragments (ACPF) | | | | | | | |
| Count | 110650 | 116997 | 110276 | 98325 | 73927 | 118318 | 108759 |
| Length (mean, nt.) | 3439 | 4200 | 5277 | 6424 | 8549 | 2926 | 4037 |
| Length (s.d., nt.) | 2972 | 3712 | 4492 | 5548 | 8224 | 1977 | 3396 |
| ACPF tagged by SQFs | | | | | | | |
| SQFs of any length | 109759 | 116259 | 109884 | 97698 | 73682 | 117251 | 104806 |
| Short SQFs | 89661 | 96633 | 94150 | 85952 | 66348 | 93930 | 87130 |
| Ranged SQFs | 106195 | 113596 | 108074 | 96466 | 72926 | 113191 | 101947 |
| Long SQFs | 55361 | 70913 | 78685 | 75169 | 60937 | 54943 | 62457 |
| Obs. PP entities (count) | | | | | | | |
| Strand polarity (+1) | 465442 | 589506 | 673197 | 697978 | 622486 | 435548 | 511058 |
| Strand polarity (−1) | 467530 | 589594 | 672274 | 698613 | 623243 | 435362 | 511484 |
| Total | 932972 | 1179100 | 1345471 | 1396591 | 1245729 | 870910 | 1022542 |
| Obs. PP definitions | 114973 | 114322 | 111550 | 107659 | 98212 | 116144 | 87550 |
| Obs. SQF-fractions | 229946 | 228644 | 223100 | 215318 | 196424 | 232288 | 175100 |
| SQFs (all) | | | | | | | |
| Count | 1865944 | 2358200 | 2690942 | 2793182 | 2491458 | 1741820 | 2045084 |
| Length (mean, nt.) | 413 | 465 | 523 | 571 | 611 | 385 | 465 |
| Length (s.d., nt.) | 456 | 514 | 578 | 641 | 688 | 410 | 516 |
| With site conflicts (%) | 6.37 | 5.68 | 5.50 | 4.85 | 4.36 | 7.18 | 6.05 |
| SQFs (all) per | | | | | | | |
| obs. SQF-fraction | | | | | | | |
| Count (mean) | 8.11 | 10.31 | 12.06 | 12.97 | 12.68 | 7.50 | 11.68 |
| Count (s.d.) | 12.30 | 14.81 | 17.70 | 23.15 | 25.12 | 7.54 | 16.73 |
| SQFs (ranged) | | | | | | | |
| Count | 1108658 | 1370126 | 1513373 | 1529178 | 1331042 | 1048675 | 1179984 |
| Length (mean, nt.) | 323 | 331 | 336 | 340 | 343 | 320 | 330 |
| Length (s.d., nt.) | 164 | 165 | 166 | 168 | 168 | 163 | 166 |
| With site conflicts (%) | 4.84 | 4.23 | 4.20 | 3.63 | 3.22 | 5.49 | 4.56 |
| SQFs (ranged) per | | | | | | | |
| obs. SQF-fraction | | | | | | | |
| Count (mean) | 5.22 | 6.46 | 7.38 | 7.84 | 7.68 | 4.85 | 7.20 |
| Count (s.d.) | 8.45 | 9.42 | 10.03 | 12.83 | 13.19 | 4.90 | 10.33 |

¹These results were obtained using the indicated search target-groups and 926 Mb of finished genomic DNA sequence data from the nuclear genome of H. sapiens. In all cases, the lower and upper bounds used to define “ranged” SQFs were 100 and 700 nt., respectively. Also, in all cases, the (5 × 4) search target groups used are capable of generating 122,880 theoretical PP definitions and 245,760 theoretical SQF-fractions.
²Legend: s.d., standard deviation; nt., nucleotides; obs., observed.

TABLE 18
Summary results for SQF analyses of mouse genomic DNA sequence data^{1,2}.

| SQF analysis ID: | 9 | 10 | 11 | 12 | 59 | 60 | 61 |
|---|---|---|---|---|---|---|---|
| Target-group ID | 1 | 2 | 3 | 4 | 5 | 6 | 7 |
| Partition fragments: | | | | | | | |
| Count | 7414 | 5292 | 4594 | 4579 | 1849 | 11587 | 13151 |
| Length (mean, nt.) | 2086 | 2865 | 3316 | 3359 | 7345 | 1374 | 1198 |
| Length (s.d., nt.) | 3074 | 3813 | 3632 | 3520 | 8438 | 1600 | 1882 |
| ACP fragments (ACPF) | | | | | | | |
| Count | 2151 | 2023 | 2125 | 2212 | 1236 | 2400 | 2262 |
| Length (mean, nt.) | 4227 | 4908 | 4856 | 4754 | 8599 | 2952 | 3434 |
| Length (s.d., nt.) | 4172 | 4778 | 4193 | 4028 | 9225 | 2334 | 3026 |
| ACPF tagged by SQFs | | | | | | | |
| SQFs of any length | 2141 | 2013 | 2123 | 2205 | 1234 | 2385 | 2182 |
| Short SQFs | 1856 | 1807 | 1886 | 1976 | 1135 | 2009 | 1861 |
| Ranged SQFs | 2091 | 1978 | 2090 | 2172 | 1231 | 2301 | 2121 |
| Long SQFs | 1302 | 1398 | 1477 | 1505 | 1020 | 1126 | 1155 |
| Obs. PP entities (count) | | | | | | | |
| Strand polarity (+1) | 11943 | 12595 | 13891 | 14249 | 11308 | 10120 | 10273 |
| Strand polarity (−1) | 12064 | 12617 | 14016 | 14425 | 11271 | 10273 | 10426 |
| Total | 24007 | 25212 | 27907 | 28674 | 22579 | 20393 | 20699 |
| Obs. PP definitions | 16388 | 16638 | 17929 | 18271 | 13447 | 15015 | 14759 |
| Obs. SQF-fractions | 32776 | 33276 | 35858 | 36542 | 26894 | 30030 | 29518 |
| SQFs (all) | | | | | | | |
| Count | 48014 | 50424 | 55814 | 57348 | 45158 | 40786 | 41398 |
| Length (mean, nt.) | 445 | 466 | 471 | 467 | 551 | 378 | 409 |
| Length s.d.,nt. | 500 | 508 | 513 | 511 | 613 | 421 | 447 |
| With site conflicts (%) | 6.13 | 5.57 | 5.53 | 5.47 | 4.72 | 7.50 | 6.77 |
| SQFs (all) per | | | | | | | |
| obs. SQF-fraction | | | | | | | |
| Count (mean) | 1.46 | 1.52 | 1.56 | 1.57 | 1.68 | 1.36 | 1.40 |
| Count (s.d.) | 0.99 | 1.01 | 1.12 | 1.13 | 1.33 | 0.85 | 0.92 |
| SQFs (ranged) | | | | | | | |
| Count | 28015 | 29152 | 32106 | 33103 | 24802 | 24451 | 24703 |
| Length (mean, nt.) | 325 | 330 | 330 | 329 | 340 | 313 | 322 |
| Length(s.d., nt.) | 165 | 166 | 166 | 166 | 168 | 161 | 162 |
| With site conflicts (%) | 4.53 | 4.06 | 4.16 | 3.98 | 3.54 | 5.66 | 5.18 |
| SQFs (ranged) per | | | | | | | |
| obs. SQF-fraction | | | | | | | |
| Count (mean) | 1.33 | 1.35 | 1.38 | 1.39 | 1.41 | 1.28 | 1.31 |
| Count (s.d.) | 0.81 | 0.74 | 0.92 | 0.92 | 0.88 | 0.80 | 0.90 |

¹These results were obtained using the indicated search target-groups and 16 Mb of finished genomic DNA sequence data from the nuclear genome of M. musculus. In all cases, the lower and upper bounds used to define “ranged” SQFs were 100 and 700 nt., respectively. Also, in all cases, the (5 × 4) search target groups used are capable of generating 122,880 theoretical PP definitions and 245,760 theoretical SQF-fractions.
²Legend: s.d., standard deviation; nt., nucleotides; obs., observed.

TABLE 19
Summary results for SQF analyses of fruit-fly genomic DNA sequence data^{1,2}.

| SQF analysis ID: | 17 | 18 | 19 | 20 | 75 | 76 | 77 |
|---|---|---|---|---|---|---|---|
| Target-group ID | 1 | 2 | 3 | 4 | 5 | 6 | 7 |
| Partition fragments: | | | | | | | |
| Count | 109092 | 79827 | 34753 | 22115 | 27200 | 226127 | 230245 |
| Length (mean, nt.) | 1064 | 1454 | 3339 | 5247 | 4266 | 513 | 504 |
| Length (s.d., nt.) | 1402 | 1863 | 3817 | 5825 | 4803 | 693 | 777 |
| ACP fragments (ACPF) | | | | | | | |
| Count | 11623 | 13044 | 13378 | 11813 | 12803 | 5223 | 6054 |
| Length (mean, nt.) | 3370 | 4034 | 6423 | 8384 | 7297 | 1898 | 2323 |
| Length (s.d., nt.) | 2305 | 2666 | 4366 | 6334 | 5395 | 1102 | 1448 |
| ACPF tagged by SQFs | | | | | | | |
| SQFs of any length | 11439 | 12912 | 13321 | 11756 | 12731 | 5035 | 5677 |
| Short SQFs | 9025 | 10283 | 11105 | 10128 | 10784 | 3923 | 4330 |
| Ranged SQFs | 10963 | 12472 | 13037 | 11570 | 12483 | 4560 | 5234 |
| Long SQFs | 5018 | 6929 | 9647 | 9345 | 9668 | 914 | 1541 |
| Obs. PP entities (count) | | | | | | | |
| Strand polarity (+1) | 36616 | 47826 | 67992 | 72569 | 71537 | 10604 | 12836 |
| Strand polarity (−1) | 36928 | 47691 | 68480 | 72460 | 72115 | 10676 | 12982 |
| Total | 73544 | 95517 | 136472 | 145029 | 143652 | 21280 | 25818 |
| Obs. PP definitions | 38977 | 43289 | 44611 | 41474 | 43480 | 17158 | 18505 |
| Obs. SQF-fractions | 77954 | 86578 | 89222 | 82948 | 86960 | 34316 | 37010 |
| SQFs (all) | | | | | | | |
| Count | 147088 | 191034 | 272944 | 290058 | 287304 | 42560 | 51636 |
| Length (mean, nt.) | 371 | 423 | 537 | 601 | 568 | 257 | 308 |
| Length (s.d., nt.) | 1080 | 1302 | 1330 | 1100 | 889 | 316 | 400 |
| With site conflicts (%) | 8.29 | 7.44 | 6.44 | 5.60 | 5.97 | 12.84 | 11.82 |
| SQFs (all) per | | | | | | | |
| obs. SQF-fraction | | | | | | | |
| Count (mean) | 1.89 | 2.21 | 3.06 | 3.50 | 3.30 | 1.24 | 1.40 |
| Count (s.d.) | 1.58 | 2.11 | 3.95 | 5.19 | 4.57 | 0.86 | 1.15 |
| SQFs (ranged) | | | | | | | |
| Count | 88529 | 112652 | 151812 | 155240 | 156963 | 25204 | 30872 |
| Length (mean, nt.) | 317 | 324 | 337 | 341 | 339 | 287 | 300 |
| Length (s.d., nt.) | 161 | 164 | 167 | 168 | 168 | 151 | 158 |
| With site conflicts (%) | 6.24 | 5.64 | 4.90 | 4.19 | 4.53 | 10.07 | 9.27 |
| SQFs (ranged) per | | | | | | | |
| obs. SQF-fraction | | | | | | | |
| Count (mean) | 1.56 | 1.75 | 2.25 | 2.48 | 2.38 | 1.16 | 1.27 |
| Count (s.d.) | 1.08 | 1.39 | 2.37 | 2.94 | 2.66 | 0.78 | 1.08 |

¹These results were obtained using the indicated search target-groups and 116 Mb of finished genomic DNA sequence data from the nuclear genome of D. melanogaster. In all cases, the lower and upper bounds used to define “ranged” SQFs were 100 and 700 nt., respectively. Also, in all cases, the (5 × 4) search target groups used are capable of generating 122,880 theoretical PP definitions and 245,760 theoretical SQF-fractions.
²Legend: s.d., standard deviation; nt., nucleotides; obs., observed.

TABLE 20
Summary results for SQF analyses of nematode genomic DNA sequence data^{1,2}.

| SQF analysis ID: | 26 | 27 | 28 | 29 | 91 | 92 | 93 |
|---|---|---|---|---|---|---|---|
| Target-group ID | 1 | 2 | 3 | 4 | 5 | 6 | 7 |
| Partition fragments: | | | | | | | |
| Count | 125450 | 61888 | 19689 | 19827 | 22314 | 211634 | 142120 |
| Length (mean, nt.) | 796 | 1614 | 5072 | 5038 | 4475 | 472 | 702 |
| Length (s.d., nt.) | 1615 | 2659 | 6744 | 6789 | 5958 | 1151 | 1554 |
| ACP fragments (ACPF) | | | | | | | |
| Count | 5776 | 8858 | 8787 | 8404 | 9047 | 2699 | 5772 |
| Length (mean, nt.) | 2980 | 4492 | 8946 | 9369 | 8315 | 1955 | 2938 |
| Length (s.d., nt.) | 4009 | 4623 | 8180 | 7907 | 7275 | 3544 | 3389 |
| ACPF tagged by SQFs | | | | | | | |
| SQFs of any length | 5663 | 8767 | 8756 | 8368 | 9001 | 2596 | 5552 |
| Short SQFs | 4334 | 6890 | 7455 | 7135 | 7610 | 2037 | 4173 |
| Ranged SQFs | 5367 | 8457 | 8611 | 8224 | 8846 | 2323 | 5208 |
| Long SQFs | 1821 | 4648 | 6837 | 6664 | 6910 | 385 | 1939 |
| Obs. PP entities (count) | | | | | | | |
| Strand polarity (+1) | 14515 | 30455 | 48276 | 48454 | 48310 | 4809 | 13546 |
| Strand polarity (−1) | 14520 | 30518 | 48166 | 48200 | 47970 | 4780 | 13508 |
| Total | 29035 | 60973 | 96442 | 96654 | 96280 | 9589 | 27054 |
| Obs. PP definitions | 19924 | 30882 | 31975 | 31173 | 32332 | 7838 | 17543 |
| Obs. SQF-fractions | 39848 | 61764 | 63950 | 62346 | 64664 | 15676 | 35086 |
| SQFs (all) | | | | | | | |
| Count | 58070 | 121946 | 192884 | 193308 | 192560 | 19178 | 54108 |
| Length (mean, nt.) | 349 | 450 | 617 | 640 | 611 | 246 | 355 |
| Length (s.d., nt.) | 1620 | 1316 | 1286 | 1465 | 1320 | 450 | 1312 |
| With site conflicts (%) | 9.91 | 7.16 | 5.85 | 5.36 | 5.78 | 14.33 | 11.19 |
| SQFs (all) per | | | | | | | |
| obs. SQF-fraction | | | | | | | |
| Count (mean) | 1.46 | 1.97 | 3.02 | 3.10 | 2.98 | 1.22 | 1.54 |
| Count (s.d.) | 0.94 | 1.77 | 4.21 | 4.50 | 4.06 | 0.54 | 1.01 |
| SQFs (ranged) | | | | | | | |
| Count | 35055 | 71202 | 102678 | 101769 | 103097 | 11052 | 32484 |
| Length (mean, nt.) | 307 | 326 | 340 | 341 | 339 | 282 | 310 |
| Length (s.d., nt.) | 159 | 165 | 168 | 168 | 168 | 150 | 159 |
| With site conflicts (%) | 7.47 | 5.17 | 4.34 | 3.93 | 4.29 | 11.36 | 8.99 |
| SQFs (ranged) per | | | | | | | |
| obs. SQF-fraction | | | | | | | |
| Count (mean) | 1.32 | 1.65 | 2.22 | 2.28 | 2.22 | 1.17 | 1.37 |
| Count (s.d.) | 0.72 | 1.22 | 2.46 | 2.63 | 2.41 | 0.45 | 0.76 |

¹These results were obtained using the indicated search target-groups and 100 Mb of finished genomic DNA sequence data from the nuclear genome of C. elegans. In all cases, the lower and upper bounds used to define “ranged” SQFs were 100 and 700 nt., respectively. Also, in all cases, the (5 × 4) search target groups used are capable of generating 122,880 theoretical PP definitions and 245,760 theoretical SQF-fractions.
²Legend: s.d., standard deviation; nt., nucleotides; obs., observed.

TABLE 21
Summary results for SQF analyses of yeast genomic DNA sequence data^{1,2}.

| SQF analysis ID: | 34 | 35 | 36 | 38 | 107 | 108 | 109 |
|---|---|---|---|---|---|---|---|
| Target-group ID | 1 | 2 | 3 | 4 | 5 | 6 | 7 |
| Partition fragments: | | | | | | | |
| Count | 11462 | 6649 | 3327 | 1411 | 2021 | 29343 | 13810 |
| Length (mean, nt.) | 1050 | 1787 | 3581 | 8467 | 5932 | 411 | 871 |
| Length (s.d., nt.) | 1146 | 1949 | 3691 | 8752 | 6028 | 416 | 977 |
| ACP fragments (ACPF) | | | | | | | |
| Count | 1508 | 1650 | 1581 | 1005 | 1237 | 552 | 1304 |
| Length (mean, nt.) | 2910 | 4096 | 6113 | 11242 | 8772 | 1508 | 2714 |
| Length (s.d., nt.) | 1537 | 2338 | 3861 | 8944 | 6126 | 690 | 1410 |
| ACPF tagged by SQFs | | | | | | | |
| SQFs of any length | 1484 | 1632 | 1571 | 981 | 1234 | 539 | 1248 |
| Short SQFs | 1179 | 1359 | 1366 | 913 | 1128 | 447 | 995 |
| Ranged SQFs | 1416 | 1597 | 1544 | 970 | 1218 | 487 | 1189 |
| Long SQFs | 538 | 874 | 1068 | 834 | 980 | 58 | 415 |
| Obs. PP entities (count) | | | | | | | |
| Strand polarity (+1) | 4572 | 6763 | 8608 | 7657 | 8360 | 1045 | 3444 |
| Strand polarity (−1) | 4539 | 6628 | 8240 | 7486 | 8353 | 1055 | 3483 |
| Total | 9111 | 13391 | 16848 | 15143 | 16713 | 2100 | 6927 |
| Obs. PP definitions | 7682 | 10362 | 10941 | 8780 | 10021 | 1976 | 5863 |
| Obs. SQF-fractions | 15364 | 20724 | 21882 | 17560 | 20042 | 3952 | 11726 |
| SQFs (all) | | | | | | | |
| Count | 18222 | 26782 | 33696 | 30286 | 33426 | 4200 | 13854 |
| Length (mean, nt.) | 335 | 399 | 466 | 547 | 521 | 218 | 322 |
| Length (s.d., nt.) | 350 | 429 | 503 | 592 | 577 | 241 | 333 |
| With site conflicts (%) | 9.77 | 7.63 | 7.08 | 5.61 | 6.07 | 17.05 | 13.15 |
| SQFs (all) per | | | | | | | |
| obs. SQF-fraction | | | | | | | |
| Count (mean) | 1.19 | 1.29 | 1.54 | 1.72 | 1.67 | 1.06 | 1.18 |
| Count (s.d.) | 0.79 | 0.84 | 1.29 | 1.64 | 1.54 | 0.83 | 0.92 |
| SQFs (ranged) | | | | | | | |
| Count | 11253 | 16185 | 19479 | 16686 | 18827 | 2420 | 8335 |
| Length (mean, nt.) | 313 | 323 | 334 | 337 | 339 | 277 | 315 |
| Length (s.d.,nt.) | 161 | 165 | 167 | 167 | 168 | 144 | 164 |
| With site conflicts (%) | 7.68 | 5.44 | 5.40 | 4.47 | 4.58 | 13.06 | 10.26 |
| SQFs (ranged) per | | | | | | | |
| obs. SQF-fraction | | | | | | | |
| Count (mean) | 1.13 | 1.18 | 1.33 | 1.43 | 1.40 | 1.06 | 1.12 |
| Count (s.d.) | 0.70 | 0.55 | 0.88 | 1.03 | 1.02 | 0.83 | 0.76 |

¹These results were obtained using the indicated search target-groups and 12 Mb of finished genomic DNA sequence data from the nuclear genome of S. cerevisiae. In all cases, the lower and upper bounds used to define “ranged” SQFs were 100 and 700 nt., respectively. Also, in all cases, the (5 × 4) search target groups used are capable of generating 122,880 theoretical PP definitions and 245,760 theoretical SQF-fractions.
²Legend: s.d., standard deviation; nt., nucleotides; obs., observed.

TABLE 22
Summary results for simulated SQF analyses of human genomic DNA sequence data¹.

| Target-group ID | 1 | 2 | 3 | 4 | 5 | 6 | 7 |
|---|---|---|---|---|---|---|---|
| SQF analysis ID for the | 1 | 2 | 3 | 4 | 43 | 44 | 45 |
| sequence search | | | | | | | |
| comparison | | | | | | | |
| Count (all SQFs) | | | | | | | |
| By sequence search | 1865944 | 2358200 | 2690942 | 2793182 | 2491458 | 1741820 | 2045084 |
| By simulation | 2072061 | 2934415 | 3519806 | 3670592 | 3543523 | 2148318 | 1919011 |
| Agreement (%) | 90 | 80.4 | 76.4 | 76 | 70 | 81 | 106 |
| Count (ranged SQFs) | | | | | | | |
| By sequence search | 1108658 | 1370126 | 1513373 | 1529178 | 1331042 | 1048675 | 1179984 |
| By simulation | 1262912 | 1743526 | 2022817 | 2054593 | 1930697 | 1307272 | 1173000 |
| Agreement (%) | 87.8 | 78.6 | 74.8 | 74 | 69 | 80 | 100 |
| Mean length (all SQFs) | | | | | | | |
| By sequence search | 413 | 465 | 523 | 571 | 611 | 385 | 465 |
| By simulation | 328 | 365 | 393 | 410 | 423 | 331 | 321 |
| Agreement (%) | 126 | 127 | 133 | 139 | 144 | 116 | 145 |

¹These results were obtained using the indicated search target-groups. Agreement between the sequence-based SQF analysis and the SQF simulation analysis (denominator) results are expressed as percentages.

TABLE 23
Summary results for simulated SQF analyses of yeast genomic DNA sequence data¹.

| Target-group ID | 1 | 2 | 3 | 4 | 5 | 6 | 7 |
|---|---|---|---|---|---|---|---|
| SQF analysis ID for the | 34 | 35 | 36 | 38 | 107 | 108 | 109 |
| sequence search | | | | | | | |
| comparison | | | | | | | |
| Count (all SQFs) | | | | | | | |
| By sequence search | 18222 | 26782 | 33696 | 30286 | 33426 | 4200 | 13854 |
| By simulation | 18218 | 28478 | 36246 | 32159 | 35435 | 4259 | 14634 |
| Agreement (%) | 100 | 94 | 93 | 94 | 94 | 99 | 95 |
| Count (ranged SQFs) | | | | | | | |
| By sequence search | 11253 | 16185 | 19479 | 16686 | 18827 | 2420 | 8335 |
| By simulation | 11206 | 17233 | 21136 | 17935 | 20102 | 2473 | 9003 |
| Agreement (%) | 100 | 94 | 93 | 93 | 94 | 98 | 92 |
| Mean length (all SQFs) | | | | | | | |
| By sequence search | 335 | 399 | 466 | 547 | 521 | 218 | 322 |
| By simulation | 290 | 329 | 364 | 388 | 380 | 202 | 274 |
| Agreement (%) | 116 | 121 | 128 | 141 | 137 | 108 | 118 |

¹These results were obtained using the indicated search target-groups. Agreement between the sequence-based SQF analysis and the SQF simulation analysis (denominator) results are expressed as percentages.

### Equivalents

The purpose of the above description and examples is to illustrate some embodiments of the present invention without implying any limitation. For example, different computer hardware, computer operating systems, computer network infrastructures, computer program application architectures (desktop, file-server, client-server, web-application server, etc.), transaction-processing middleware, database software, database schema, computer programming languages, computer software development tools, algorithms, and computer programming code could be used to implement and program the design, distribution of executable components (on one or more computers), information-processing logic, and ancillary functionality of the database application and other computer software that comprises part of this invention. Thus, although the present invention is fully set forth above, it will be apparent to those of ordinary skill in the art that various changes and modifications can be made to the form and details of the invention without departing from the spirit or scope of the invention as defined by the appended claims.

The ability of the present invention, and any future embodiments thereof, to interface with external databases, computer software, analytical tools, or instrumentation and the like is understood to be in the purview of one of ordinary skill in the art.

What is claimed is:

1. A method for characterizing a set of strings, said method comprising:
a) receiving the set of strings comprising process-pattern containing substrings;
b) defining a series of search target string patterns effective for searching the set of strings; and
c) processing the set of strings through an ordered series of search steps each search step being specific for one of the search classes and involving an attempted discovery of an appropriate search target site to define a delimited search region for the next step, thereby characterizing the set of strings.

2. The method of claim 1, wherein the series of search target patterns is determined by identifying permutations of a search target group comprising an ordered set of search targets including a partition search target followed by major classes of ranked member search targets, the partition search target effective for determining partition fragments in the set of strings.

3. The method of claim 2, wherein the set of strings is a set of polynucleotides, the number of classes is between 3 and 9, the number of ranked member targets in a class is less than or equal to 9, and a search target in the search target group comprises a distinct recognition sequence for a cleavage effector.

4. The method of claim 3, wherein processing the set of strings comprises identifying within the partition fragments qualifying fragments including a member of each major class and querying the qualifying fragments according to the process, wherein the process uses a defined polarity and extremum condition to identify process-pattern containing substrings containing one search site from each class and to identify structured query fragments within the process-pattern containing substrings.

5. The method of claim 4, wherein for each step of the process the search target chosen to contribute to the pattern is the highest-ranked member of a search class according to the search target pattern.

6. The method of claim 5, wherein the method is performed using a computer algorithm.

7. The method of claim 6, wherein the search target group comprises a symmetrically descending array of search targets.

8. The method of claim 5, wherein
a) the set of strings is a physical sample of polynucleotides;
b) the structured query fragments are physical polynucleotide fragments that remain after the processing the set of strings; and
c) the method further comprises detecting the structured query fragments.

9. A method for analyzing a set of polynucleotides, wherein the method comprises:
a) identifying electronic structured query fragments, wherein the identifying comprises:
i) electronically receiving a set of strings representing the set of polynucleotides;
ii) defining a series of search target string patterns that are identical to a series of recognition site patterns cleavage effectors; and
iii) identifying structured query fragment strings within the set of strings by identifying substrings that remain after processing the set of strings through a series of step-wise delimitation processes comprising identifying target strings flanked by search target strings and using the target strings for a next pre-emptive target search according to the series of search target string patterns; and
b) isolating physical structured query fragments, wherein the isolating comprises:
i) providing the set of polynucleotides; and
ii) isolating physical structured query fragments within the set of polynucleotides by isolating fragments that remain after processing the set of polynucleotides through a series of step-wise delimitation processes comprising cleaving the set of polynucleotides with a cleavage effector to form a set of polynucleotide fragments including target polynucleotide fragments, and retaining only the target polynucleotide fragments for a next pre-emptive cleavage according to each recognition site pattern of the series of recognition site patterns; and
c) comparing the electronic structured query fragments to the physical structured query fragments, thereby analyzing the set of polynucleotides.

10. A laboratory method for isolating and characterizing a set of polynucleotides, said method comprising:
a) providing the set of polynucleotides;
b) defining a series of recognition site patterns for sequence-specific polynucleotide cleavage reagents; and
c) isolating physical structured query fragments within the set of polynucleotides by isolating fragments that remain after processing the set of polynucleotides through a series of step-wise delimitation processes comprising cleaving the set of polynucleotides with a polynucleotide cleavage reagent to form a set of polynucleotide fragments including selected polynucleotide fragments, and retaining only the selected polynucleotide fragments for a next pre-emptive cleavage according to each recognition site pattern of the series of recognition site patterns,
d) detecting the physical structured query fragments, thereby isolating and characterizing the physical structure query fragments.

11. A method for characterizing a set of strings comprising process-pattern containing substrings, said method comprising:
a) receiving the set of strings;
b) defining a series of search target string patterns of search targets, the search target string patterns being effective for searching the set of strings; and
c) defining a process for identifying the process-pattern containing substrings based on a selected arrangement of search targets within a search target string pattern; and
d) performing the process to identify the process-pattern containing substrings within the set of strings for each search target pattern in the series of search target patterns, thereby characterizing the set of strings.

12. A method for characterizing sets of strings, the method comprising:
(a) receiving one or more sets of strings of any length, wherein may be found occurrences of relatively short search-target-strings of interest; and where one or more of the short search-target-strings are used to define a distinct search target; and where several distinct search targets or targets are assembled into structured entities known as search target groups, where a search target group is comprised of: (i) a partition search target that is used to partition the sets of strings under study into substrings or partition fragments bounded by consecutive occurrences of the partition search target; and (ii) a small array of a limited number M of major classes or ordered sets of search targets, where each major class is comprised of a limited number of ranked member search targets; and where a search target group or target group, or two or more search target groups or target groups of distinct composition or structure, may be used to characterize search target group-defined substrings found within the sets of strings under study;
(b) using the structure and composition of a search target group with M major classes to define a search process comprised of a series of M search steps that are to be effected within each of the partition fragments obtained, from the sets of strings under study, using the partition search target of the target group; and where the search process defines patterns, of occurrence within the partition fragments of search targets that are members of the target group; and where partition fragments or regions therein may be characterized by the occurrence therein of instances, of the process-patterns that may be defined by the structure and composition of the target group; and
(c) using the structure and composition of a search target group with M major classes to effect a search process comprised of a series of M search steps within each of the partition fragments obtained, from the sets of strings under study, using the partition search target of the target group; and where the search process results in the detection of process-pattern entities, where each process-pattern entity is comprised of a pattern of M search target sites, which together include a search target site representing one member of each of the M major classes in the target group; and where each of the sites must be present and where sites representing higher-ranked members of the same major class must be absent within the relevant search area for the major class in the partition fragment; and where the process-pattern entities are obtained as a result of a stepwise search and delimitation process after each site is found that restricts the region of the partition fragment where the next class-specific target-search occurs; and where partition fragments or regions therein may be characterized by the occurrence therein of process-pattern entities, where the process-pattern entities represent instances of the process-patterns that may be defined by the structure and composition of the target group; and where partition fragments or regions therein may be characterized by the occurrence therein of structured query fragments (SQFS) that are fragments bounded any two search target sites in a process-pattern entity, and whose lengths can be calculated by the positions of the constituent sites that comprise the process-pattern entity wherein the SQFs are found; and where the SQFs of particular interest are typically the SQFs bounded by the last two search target sites detected in the identification of a process-pattern entity.

13. The method of claim 12, wherein a search target group with M major classes is used, and the process-patterns are defined using one or more of the M! permutations of the M major classes of search targets in the search target group, where each major-class permutation defines the order with which the major classes of the target group are used to search the partition fragments for the presence of process-pattern entities that may be defined by the structure and composition of the target group.

14. The method of claim 13, wherein the method is performed using a computer software algorithm.

15. The method of claim 12, wherein the sets of strings represent sets of biopolymer sequence data.

16. The method of claim 13, wherein the sets of strings represent sets of polynucleotide sequence data.

17. The method of claim 14, wherein the sets of strings represent sets of polypeptide sequence data.

18. The method of claim 15, wherein process-pattern entities and SQFs detected in sets of strings of the same biopolymer sequence type, and obtained using the same search target group, are used to compare the sets of biopolymer sequence data and establish sequence similarity or homologous, paralogous, or orthologous sequence relationships between partition fragments or regions therein contained within one or more of the sets of strings under study.

19. The methods of claim 18, where the identification of sequence similarity or homologous, paralogous, or orthologous sequence relationships may lead to the identification of genes, gene regulatory regions, or other chromosomal, genetic, or genomic regions of interest.

20. The methods of claim 18, where the identification of sequence similarity or homologous, paralogous, or orthologous sequence relationships may lead to the identification of polypeptide structural elements or functional capabilities of interest.

21. The method of claim 15, wherein the major search targets in a search target group comprise a symmetrically descending array of search targets, such that regardless of class, each member search target of the same rank in the array has approximately the same mean recurrence length in the set of strings under study; and within each major class, the member search targets are ranked in descending order based on the ranking in descending order of their mean fragment lengths in the set of strings under study.

22. The method of claim 15, wherein the number M of major classes in the search target groups used is between 3 and 9, and the number of ranked member search targets in each major class is between 1 and 9.

23. The method of claim 16, wherein each search target in a search target group represents a distinct recognition sequence for a sequence-specific, polynucleotide cleavage effector.

24. The method of claim 23, wherein each search target in a search target group represents a distinct recognition sequence for a Type II restriction endonuclease.

25. A laboratory method for the physical characterization of a sample of polynucleotides of the same general type, the method comprising:
(a) obtaining a sample of polynucleotides, wherein may be found occurrences of relatively short recognition sequences for sequence-specific, polynucleotide cleavage effectors of interest; and where each of the recognition sequences is used to define a distinct search target for their respective sequence-specific polynucleotide cleavage effector; and where several distinct search targets or targets are assembled into structured entities known as search target groups, where a search target group is comprised of (i) a partition search target whose sequence-specific polynucleotide cleavage effector is used to cleave the polynucleotide sample under study into partition fragments bounded by consecutive occurrences of the partition search target; and (ii) a small array of a limited number M of major classes or ordered sets of search targets, where each major class is comprised of a limited number of ranked member search targets; and where a target group may be used for the physical characterization of a sample of polynucleotides, or two or more target groups of distinct composition or structure may each be used separately for the physical characterization of separate, identical samples of polynucleotides, where the physical characterization is summarized by following series of steps, comprising:
(i) blocking of random termini of the polynucleotide fragments that comprise the sample of polynucleotides, in order to prevent addition of derivatizing reagents thereto;
(ii) cleavage of the sample of polynucleotides using the sequence-specific, polynucleotide cleavage effector whose recognition sequence represents the partition search target;
(iii) derivatization of non-random termini of the partition fragments obtained in the previous step, using a derivatizing reagent that allows for their subsequent termini-specific immobilization, where the non-random termini were generated by the action of the sequence-specific, polynucleotide cleavage effector whose recognition sequence represents the partition search target;
(iv) termini-specific immobilization of the derivatized polynucleotide fragments on a reactively appropriate solid support, where the reactively appropriate solid support is one that reacts appropriately with, and thereby permits the termini-specific immobilization of, the derivatized polynucleotide fragments;
(v) blocking of any unreacted sites on the solid support;
(vi) iterated, sequence-specific cleavage of the immobilized partition fragments using, in rank order, the sequence-specific polynucleotide cleavage effectors representing the ranked members of the first major class of search targets to be used; and where after each reaction, the product obtained thereby in solution contains liberated polynucleotide fragments of interest and is isolated for subsequent use; and where the immobilized substrate polynucleotide fragments that remain on the solid support are available for subsequent iterated, sequence-specific cleavage reactions using, in rank order, the remaining members of the same major class;
(vii) recursive immobilization of the isolated reaction products obtained from the previous step, where the isolated products are immobilized via their termini on physically distinct, reactively appropriate solid supports as described earlier; and where any unreacted sites on the solid support are subsequently blocked as described earlier; and where the freshly immobilized polynucleotide fragments on the blocked support are derivatized at their distal-to-the-support termini to permit the subsequent recursive immobilization of fragments that have derivatized termini and are liberated by the activity of a sequence-specific polynucleotide cleavage effector in the next step;
(viii) iterated, sequence-specific cleavage of the immobilized fragments from the previous step, using, in rank order, the sequence-specific polynucleotide cleavage effectors representing the ranked members of the next major class of search targets to be used; and where after each reaction, the product obtained thereby in solution contains liberated polynucleotide fragments of interest and is isolated for subsequent use; and where the immobilized substrate polynucleotide fragments that remain on the solid support are available for subsequent iterated, sequence-specific cleavage reactions using, in rank order, the remaining members of the same major class;
(ix) the stepwise generation of progressively expanding, process-pattern defined subsets of polynucleotide fragments, where the fragments are generated by the repeated execution of the previous two steps, and where each of the major classes that comprise the search target group used for the analysis are employed, resulting in the isolation of process-pattern defined structured query fragment (SQF) fractions that for a given SQF fraction contain all of the SQFs that may be obtained from the sample of polynucleotides using the process-pattern definition associated with the SQF fraction; and where the SQFs present in a given SQF fraction represent fragments bounded by the last two search target sites that are cleaved in all of the process-pattern entities that share the process-pattern definition associated with the SQF fraction, and that may be obtained from the sample of polynucleotides.

26. The method of claim 25, wherein a search target group with M major classes is used, and the process-patterns are defined using one or more of the M! permutations of the M major classes of search targets in the search target group, where each major-class permutation defines the order with which the major classes of the target group are used to obtain process-pattern defined SQF fractions from the sample of polynucleotides.

27. The method of claim 26, wherein the resolution and detection of individual SQFs within a process-pattern defined SQF fraction is effected by an analytical technique that may or may not require end-labeling of the immobilized polynucleotide fragments prior to their iterated cleavage using the last major class to be used in the analysis of the sample of polynucleotides; and where the analytical technique provides a length estimate associated with each SQF resolved and detected by the analytical technique.

28. The method of claim 17, where the objective is to identify physical SQFs obtainable from the sample of polynucleotides that are not obtainable from the set of polynucleotide sequence data that purportedly describes the sample of polynucleotides, and where the physical SQFs may be useful for the generation of polynucleotide sequence data that may address deficiencies in the completeness of the polynucleotide sequence data set.

29. The method of claim 26, where one or more of the SQF fractions obtained is used for the establishment of molecular clones of the SQFs therein.

30. The method of claim 25, wherein each search target in a search target group represents a distinct recognition sequence for a Type II restriction endonuclease, and where the sample of polynucleotides is double-stranded DNA.

31. A method for characterizing sets of strings, the method comprising:
(a) receiving one or more sets of strings of any length, wherein may be found occurrences of relatively short search-target-strings of interest; and where one or more of the short search-target-strings are used to define a distinct search target; and where several distinct search targets or targets are assembled into structured entities known as search target groups, where a search target group is comprised of: (i) a partition search target that is used to partition the sets of strings under study into substrings or partition fragments bounded by consecutive occurrences of the partition search target; and (ii) a small array of a limited number M of major classes or ordered sets of search targets, where each major class is comprised of a limited number of ranked member search targets; and where a search target group or target group, or two or more search target groups or target groups of distinct composition or structure, may be used to characterize search target group-defined substrings found within the sets of strings under study;
(b) using the structure and composition of a search target group with M major classes to define a search process comprised of a series of M search steps that are to be effected within each of the partition fragments obtained, from the sets of strings under study, using the partition search target of the target group; and where the search process defines patterns of occurrence within the partition fragments of search targets that are members of the target group; and where partition fragments or regions therein may be characterized by the occurrence therein of instances of the process-patterns that may be defined by the structure and composition of the target group or using a search target group with M major classes, and defining process-patterns using all of the M! permutations of the M major classes of search targets in the search target group, where each major-class permutation defines the order with which the major classes of the target group are used;
(c) obtaining or estimating mean recurrence length or mean fragment length data for each search target used in the search target group, where the mean fragment length data is for the search targets in the set of strings used in the analysis;
(d) obtaining or estimating the overall length of the set of strings used in the analysis;
(e) assuming that the distribution of the fragment length between consecutive occurrences of each search target used in the search target group may be approximated by the exponential distribution;
(f) using the properties of the exponential distribution, together with the mean fragment length data and the overall length of the set of strings, to derive a simple, recursive calculation method to estimate the following for a given set of strings under study and for each of the process-patterns that are defined by the search target group used for the analysis: (i) the number of SQFs of any size; (ii) the number of SQFs within a given size range; and (iii) the mean fragment length of SQFs of any size.

32. The method of claim 31, wherein the method is performed using a computer software algorithm.

33. The method of claim 31, wherein the sets of strings represent sets of biopolymer sequence data.

34. The method of claim 32, wherein the sets of strings represent sets of polynucleotide sequence data.
